(19) United States
(12) Patent Application Publication
(10) Pub. No.: US 2025/0367121 A1
(43) Pub. Date: Dec. 4, 2025

(54) DOSAGE FORMS COMPRISING ACTIVE PHARMACEUTICAL INGREDIENTS

(71) Applicant: AXSOME THERAPEUTICS, INC., New York, NY (US)

(72) Inventor: Herriot Tabuteau, New York, NY (US)

(21) Appl. No.: 19/266,899

(22) Filed: Jul. 11, 2025

Related U.S. Application Data
(63) Continuation of application No. 17/280,379, filed on Oct. 29, 2021.
(60) Provisional application No. 62/738,833, filed on Sep. 28, 2018.
(60) Provisional application No. 62/842,394, filed on May 2, 2019.

(51) Int. Cl. A61K 9/20; A61K 9/24; A61K 31/192; A61K 31/4045; A61K 31/4184; A61K 31/4196; A61K 31/5415; A61K 47/69

(52) U.S. Cl. CPC A61K 9/205; A61K 9/2009; A61K 9/209; A61K 31/192; A61K 31/4045; A61K 31/4184; A61K 31/4196; A61K 31/5415; A61K 47/6951

(57) ABSTRACT

Disclosed herein are dosage forms comprising a combination of: 1) an active pharmaceutical ingredient (API), 2) a cyclodextrin, and 3) a bicarbonate. These compositions may be orally administered, for example, to improve the bioavailability or pharmacokinetics of the drug for the treatment of conditions such as pain.

### CROSS-REFERENCE TO RELATED APPLICATIONS

This application is a continuation of U.S. patent application Ser. No. 17/280,379, filed Oct. 29, 2021; which is a National Stage of International Patent Application No. PCT/US2019/053167, filed Sep. 26, 2019; which claims the benefit of U.S. Provisional Patent Application Nos. 62/738,833, filed Sep. 28, 2018, and 62/842,394, filed May 2, 2019; all of which are incorporated by reference herein in their entirety.

### BACKGROUND

There continues to be a need for dosage forms that improve the pharmacokinetic properties and/or bioavailability of active pharmaceutic agents.

### SUMMARY

This disclosure relates to the use of a bicarbonate and/or a cyclodextrin, such as sulfobutylether β-cyclodextrin (SBEβCD), to improve the pharmacokinetics or bioavailability of a drug or an active pharmaceutical ingredient (API).

For example, some embodiments include dosage forms comprising a drug or API in combination with a cyclodextrin (optionally as an inclusion complex of the drug and the cyclodextrin), and/or a bicarbonate, and methods of treatment using the dosage form.

Some embodiments include a dosage form comprising a combination of: 1) an active pharmaceutical ingredient, 2) a cyclodextrin, and 3) a bicarbonate.

Some embodiments include a method of improving the pharmacokinetics of a drug or an active API, comprising orally administering a dosage form described herein to a mammal or a human being in need of treatment with the drug or API.

Some embodiments include a method of improving the dissolution and/or oral bioavailability of a drug or an active API, comprising orally administering a dosage form described herein to a mammal or a human being in need of treatment with the drug or API.

Some embodiments include a method of treating a disease or condition, comprising orally administering a dosage form described herein to a mammal or a human being in need thereof.

Some embodiments include a dosage form that is a solid oral dosage form having improved dissolution of the API in a human being as compared to a first reference dosage form that: 1) contains the same amount of the API, 2) does not contain a cyclodextrin, and 3) does not contain a bicarbonate, when administered to the human being.

Some embodiments include a dosage form that is a solid oral dosage form having a shorter Tₘₐₓ of the API in a human being as compared to a first reference dosage form that: 1) contains the same amount of the API, 2) does not contain a cyclodextrin, and 3) does not contain a bicarbonate, when administered to the human being.

Some embodiments include a dosage form that is a solid oral dosage form having improved dissolution of the API in a human being as compared to a second reference dosage form that: 1) contains the same amount of the API, 2) contains the same amount of the cyclodextrin, and 3) does not contain a bicarbonate, when administered to the human being.

Some embodiments include a dosage form that is a solid oral dosage form having improved bioavailability of the API in a human being as compared to a first reference dosage form that: 1) contains the same amount of the API, 2) does not contain a cyclodextrin, and 3) does not contain a bicarbonate, when administered to the human being.

Some embodiments include a dosage form that is a solid oral dosage form having improved bioavailability of the API in a human being as compared to a second reference dosage form that: 1) contains the same amount of the API, 2) contains the same amount of the cyclodextrin, and 3) does not contain a bicarbonate, when administered to the human being.

### BRIEF DESCRIPTION OF THE DRAWINGS

FIG. 1 is a depiction of the results described in Example 2 and contained in Table 8.

FIG. 2 is another depiction of the results described in Example 2 and contained in Table 8.

FIG. 3 is another depiction of the results described in Example 2 and contained in Table 8.

FIG. 4 is another depiction of the results described in Example 2 and contained in Table 8.

FIG. 5 is another depiction of the results described in Example 2 and contained in Table 8.

FIG. 6 is another depiction of the results described in Example 2 and contained in Table 8.

FIG. 7 is another depiction of the results described in Example 2 and contained in Table 8.

FIG. 8 is another depiction of the results described in Example 2 and contained in Table 8.

FIG. 9 is another depiction of the results described in Example 2 and contained in Table 8.

FIG. 10 is another depiction of the results described in Example 2 and contained in Table 8.

FIG. 11 is a plot of meloxicam plasma concentration at various time points over the first 24 hours for an embodiment of a dosage form described herein and a commercially available meloxicam dosage form.

### DETAILED DESCRIPTION

Many drugs and active pharmaceutical agents (API) have poor aqueous solubility which may reduce bioavailability and slow the onset of action. One method of increasing the solubility and bioavailability of a drug or an API is through the use of cyclodextrins and/or bicarbonates.

Generally, this may be accomplished using a dosage form, such as an oral dosage form, containing a drug or an active pharmaceutical ingredient (API) a cyclodextrin (optionally in an inclusion complex), and/or 2) a bicarbonate. Administering this type of dosage form to a patient may increase the bioavailability of the drug or API in the patient or increase the rate at which the drug or API becomes bioavailable or increase the rate at which the plasma concentration of the API or the drug increases. For example, the API or the drug may have a shorter Tₘₐₓ or may have an increased Cₘₐₓ or area under the plasma concentration curve (AUC) as a result of the administration of this type of dosage form.

A dosage form may be given enterally including, but not limited to, oral, sublingual, or rectal delivery, or parenterally including, but not limited to, intravenous, intramuscular, intranasal, or subcutaneous delivery.

For some methods, administration of the dosage form may achieve a reduction in pain or another symptom that lasts at least about one hour, at least about two hours, at least about three hours, at least about four hours, at least about six hours, at least about eight hours, at least about twelve hours, about 8 to about 24 hours, or about 24 hours. In other embodiments, administration of the dosage form may achieve a reduction in pain or another symptom that is observed at about 10 minutes, at about 30 minutes, at about one hour, at about two hours, at about three hours, at about four hours, at about five hours, at about six hours, at or less than about 5, 10, 15, 20, 25, 30, 35, 40, 45, 50, or 60 minutes, at two hours or less, at three hours or less, or other time period bound by these ranges, after administration of the dosage form.

A human being that is treated for a disease or condition with any of the dosage forms described herein may be of any age. For example the person may have an age of about 10-90 years, about 20-80 years, about 30-75 years, about 40-70 years, about 1-16 years, about 80-95 years, about 18 years or more, about 20 years or more, about 25 years or more, about 30 years or more, about 40 years or more, about 45 years or more, about 50 years or more, about 55 years or more, about 60 years or more, about 65 years or more, or any other age in a range bounded by, or between, any of these values.

In some embodiments, a human being that is treated for a disease or condition with a dosage form has suffered from the pain or condition associated with the pain for at least 1 day, at least one week, at least 2 weeks, at least 1 month, at least 6 weeks, at least 2 months, at least 3 months, at least 6 months, at least 1 year, at least 5 years, at least 10 years, at least 15 years, at least 20 years, at least 30 years, at least 40 years, at least 50 years or any duration in a range bounded by, or between, any of these values.

A cyclodextrin used in a dosage form with a drug could include a cyclodextrin, a cyclodextrin derivative, and/or a salt thereof. Cyclodextrins (also known as cycloamyloses) are generally cyclic polysaccharides which form a bucket-like shape. Cyclodextrins help to increase bioavailability of other molecules because cyclodextrins are hydrophobic on the inside and hydrophilic on the outside which helps to facilitate the transport of hydrophobic molecules to a hydrophilic medium. The naturally occurring cyclodextrins include six, seven, and eight glucose units (α, β, and γ-cyclodextrin, respectively). However, synthetic cyclodextrins containing more or less glucose units are possible. In aqueous solutions, cyclodextrins can form complexes (i.e., an inclusion complex) with drugs by incorporating the drug into the center/hydrophobic portion of the cyclodextrin ring; although cyclodextrins are also known to aggregate around a drug in a micelle-type structure. This ability of cyclodextrins may allow them to act as carriers of less soluble drugs to increase the drugs' bioavailability.

An inclusion complex of drug and cyclodextrin may be more water-soluble relative to the non-complexed drug. The cyclodextrin may be a naturally-occurring cyclodextrin (e.g., α, β, or γ-cyclodextrins) or a synthetic cyclodextrin. In some embodiments, α-cyclodextrins, derivatives, or salts thereof may be used. α-Cyclodextrins may include, but are not limited to, (2,3,6-tri-O-acetyl)-α-cyclodextrin, (2,3,6-tri-O-methyl)-α-cyclodextrin, (2,3,6-tri-O-octyl)-α-cyclodextrin, 6-bromo-6-deoxy-α-cyclodextrin, 6-iodo-6-deoxy-α-cyclodextrin, (6-O-tertbutyl-dimethylsilyl)-α-cyclodextrin, butyl-α-cyclodextrin, succinyl-α-cyclodextrin, (2-hydroxypropyl)-α-cyclodextrin, or combinations thereof.

In some embodiments, β-cyclodextrins, derivatives, or salts thereof may be used. β-cyclodextrins may include, but are not limited to, hydroxypropyl-β-cyclodextrin, 6-monodeoxy-6-monoamino-β-cyclodextrin, glucosyl-β-cyclodextrin, maltosyl-β-cyclodextrin, 6-O-α-D-glucosyl-β-cyclodextrin, 6-O-α-maltosyl-β-cyclodextrin, 6-azido-6-deoxy-β-cyclodextrin, (2,3-di-O-acetyl-6-O-sulfo)-β-cyclodextrin, methyl-β-cyclodextrin, dimethyl-β-cyclodextrin (DMBCD), trimethyl-β-cyclodextrin (TMBCD), (2,3-di-O-methyl-6-O-sulfo)-β-cyclodextrin, (2,6-di-O-methyl)-β-cyclodextrin, (2,6-di-O-ethyl)-β-cyclodextrin, (2,3,6-tri-O-methyl)-β-cyclodextrin, (2,3,6-tri-O-acetyl)-β-cyclodextrin,-(2,3,6-tri-O-benzoyl)-β-cyclodextrin, (2,3,6-tri-O-ethyl)-β-cyclodextrin, 6-iodo-6-deoxy-β-cyclodextrin, 6-(dimethyl-tert-butylsilyl)-6-deoxy-β-cyclodextrin, 6-bromo-6-deoxy-β-cyclodextrin, monoacetyl-β-cyclodextrin, diacetyl-β-cyclodextrin, triacetyl-β-cyclodextrin, (3-O-acetyl-2,6-di-O-methyl)-β-cyclodextrin, (6-O-maltosyl)-β-cyclodextrin, (6-O-sulfo)-β-cyclodextrin, (6-O-t-butyldimethylsilyl-2,3-di-O-acetyl)-β-cyclodextrin, succinyl-(2-hydroxypropyl)-β-cyclodextrin, (2,6-di-O-)ethyl-β-cyclodextrin, (2-carboxyethyl)-β-cyclodextrin (CMEβCD), hydroxyethyl-β-cyclodextrin (HEβCD), (2-hydroxypropyl)-β-cyclodextrin, (2-hydroxypropyl)-β-cyclodextrin (HPβCD), (3-hydroxypropyl)-β-cyclodextrin (3HPβCD), (2,3-hydroxypropyl)-β-cyclodextrin (DHPβCD), butyl-β-cyclodextrin, methyl-β-cyclodextrin, silyl((6-O-tert-butyldimethyl)-2,3,-di-O-acetyl)-β-cyclodextrin, succinyl-β-cyclodextrin, (2-hydroxyisobutyl)-β-cyclodextrin, randomly methylated-β-cyclodextrin, branched-β-cyclodextrin, or combinations thereof.

In other embodiments, a β-cyclodextrin may be a sulfoalkyl ether cyclodextrin, derivative, or salt thereof. Examples of sulfoalkyl ether cyclodextrin derivatives may include, but are not limited to, sulfobutyl ether-β-cyclodextrin (e.g., SBEβCD, betadex, CAPTISOL®). In some embodiments, a SBEβCD may have about 4-8, about 5-8, about 4-7, about 6-7, or about 6.5 sulfobutyl ether groups per cyclodextrin molecule.

In some embodiments, γ-cyclodextrins, derivatives, or salts thereof may be used. γ-cyclodextrins may include carboxymethyl-γ-cyclodextrin, (2,3,6-tri-O-acetyl)-γ-cyclodextrin, (2,3,6-tri-O-methyl)-γ-cyclodextrin, (2,6-di-O-pentyl)-γ-cyclodextrin, 6-(dimethyl-tert-butylsilyl)-6-deoxy-γ-cyclodextrin, 6-bromo-6-deoxy-γ-cyclodextrin, 6-iodo-6-deoxy-γ-cyclodextrin, (6-O-t-butyldimethylsilyl)-γ-cyclodextrin, succinyl-γ-cyclodextrin, hydroxypropyl-γ-cyclodextrin, (2-hydroxypropyl)-γ-cyclodextrin, acetyl-γ-cyclodextrin, butyl-γ-cyclodextrin, or combinations thereof.

In some embodiments, the dosage form may include a bicarbonate, such as sodium bicarbonate, potassium bicarbonate, etc. A bicarbonate may help to increase the pharmacokinetics or bioavailability of a drug or API, such as an API in Table 2.

Unless otherwise indicated, any reference to a compound herein, such as a drug or an API in Table 2, by name, or any other means, includes pharmaceutically acceptable salts, alternate solid forms, such as polymorphs, solvates, hydrates, enantiomers, tautomers, deuterium-modified forms, or any other chemical species, such as precursors, prodrugs, or any other chemical species that may rapidly convert to a compound described herein under conditions in which the compounds are used as described herein.

In some embodiments, use of a cyclodextrin or a bicarbonate may improve the oral bioavailability (e.g. a higher Cₘₐₓ and/or higher AUC) of a drug (such as an API in Table 2) in a subject (human or animal), by at least about 10%, at least about 20%, at least about 30%, at least about 40%, at least about 50%, at least about 60%, at least about 70%, at least about 80%, at least about 90%, up to about 100%, up to about 200%, or any amount in a range bounded by, or between, any of these values as compared to administration of the drug without the bicarbonate, without the cyclodextrin, or without the bicarbonate and without the cyclodextrin.

In some embodiments, use of a cyclodextrin or a bicarbonate may improve the oral bioavailability (e.g. a higher Cₘₐₓ and/or higher AUC) of a drug (such as an API in Table 2) in subject (human or animal) by at least about 10%, at least about 20%, at least about 30%, at least about 40%, at least about 50%, at least about 60%, at least about 70%, at least about 80%, at least about 90%, up to about 100%, up to about 200%, or any amount in a range bounded by, or between, any of these values as compared to administration of the drug without the bicarbonate, without the cyclodextrin, or without the bicarbonate and without the cyclodextrin.

Due to the improved bioavailability as described above, the dosage form may contain, or a subject may receive, on a molar basis, less of the drug, than would otherwise be administered of the drug alone. For example, a dosage form may contain, or a mammal may receive, at least about 10 mole % less, at least about 20 mole % less, at least about 30 mole % less, at least about 40 mole % less, at least about 50 mole % less, at least about 60 mole % less, at least about 70 mole % less, at least about 80 mole % less, at least about 85 mole % less, and/or up to about 90 mole % less, 95 mole % less, or 98 mole % less than would otherwise be administered without the bicarbonate, without the cyclodextrin, or without the bicarbonate and without the cyclodextrin.

In some embodiments, a dosage form may contain a drug or API in Table 2 in an amount of about 0.001-0.2 mg, about 0.01-0.2 mg, about 0.1-0.2 mg, about 0.2-0.3 mg, about 0.3-0.4 mg, about 0.4-0.5 mg, about 0.5-0.6 mg, about 0.6-0.7 mg, about 0.7-0.8 mg, about 0.9-1 mg, about 0.01-1 mg, about 0.001-0.5 mg, about 0.5-1 mg, about 0.1-1 mg, about 1-2 mg, about 2-3 mg, about 3-4 mg, about 4-5 mg, about 5-6 mg, about 6-7 mg, about 7-8 mg, about 8-9 mg, about 9-10 mg, about 0.001-5 mg, about 5-10 mg, about 0.001-10 mg, about 10-20 mg, about 20-30 mg, about 30-40 mg, about 40-50 mg, about 50-60 mg, about 60-70 mg, about 70-80 mg, about 80-90 mg, about 90-100 mg, about 10-100 mg, about 0.001-50 mg, about 50-100 mg, about 100-200 mg, about 200-300 mg, about 300-400 mg, about 400-500 mg, about 500-600 mg, about 600-700 mg, about 700-800 mg, about 800-900 mg, about 900-1,000 mg, about 100-1000 mg, about 0.001-500 mg, 500-1,000 mg, or about 0.001-1,000 mg. These doses may be repeated regularly, such as 1, 2, 3, or 4 times a day, or repeated at an interval of about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 1-2 months, about 4 weeks, about 6 weeks, about 2-3 months, about 3-4 months, about 4-5 months, about 5-6 months, about 6-7 months, about 7-8 months, about 8-9 months, about 9-10 months, about 10-11 months, about 11-12 months, about 1-2 years, or about 2 years, etc. The treatment may be continued for a period of about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 1-2 months, about 4 weeks, about 6 weeks, about 2-3 months, about 3-4 months, about 4-5 months, about 5-6 months, about 6-7 months, about 7-8 months, about 8-9 months, about 9-10 months, about 10-11 months, about 11-12 months, about 1-2 years, about 2-5 years, about 5-10 years, about 10-15 years, about 15-20 years, or longer.

For some dosage forms, a drug (such as an API in Table 2) forms a complex with the substituted-β-cyclodextrin or other cyclodextrin which may be formulated into a solid dosage form. Such a dosage form may be suitable for oral administration. A drug-cyclodextrin inclusion complex may also be dissolved in water or another solvent to form a parenteral formulation. However, physical mixtures of drug and the substituted-β-cyclodextrin or other cyclodextrins that are not inclusion complexes may also be used in oral or parenteral dosage forms.

Formation of an inclusion complex of a drug (such as an API in Table 2) and a cyclodextrin may help to improve the properties of a dosage form. For some inclusion complexes, the drug and the cyclodextrin (e.g., SBEβCD) may have a molar ratio of about 0.5-2 (a molar ratio of 0.5 is 0.5 moles of the drug to 1 mole of cyclodextrin), about 0.5-0.7, about 0.6-0.8, about 0.7-0.9, about 0.8-1, about 0.9-1.1, about 1-1.2, about 1.1-1.3, about 1.2-1.4, about 1.3-1.5, about 1.4-1.6, about 1.5-1.7, about 1.6-1.8, about 1.7-1.9, about 1.8-2, about 1.9-2.1, about 2-2.2, about 0.8-1.2, about 1, such as about 0.5-1.5 or about 0.8-1.2, about 1-2, such as about 1-1.5 or about 1.5-2, or any ratio in a range bounded by any of these values.

In some embodiments, an inclusion complex is formed by (1) mixing a homogeneous solution of a drug or an API with a homogeneous solution of the cyclodextrin to form a homogeneous solution of the drug and the cyclodextrin, and (2) removing or evaporating the solvent of the homogeneous solution of the drug and the cyclodextrin to form the complex comprising the inclusion complex of the drug in a cyclodextrin. In some embodiments, the solutions can be pH-adjusted aqueous solutions. The pH can be adjusted using a buffering agent. In some embodiments, the solvent can be removed or evaporated by lyophilization, spray drying, or any other means that is suitable. In some embodiments, the solvent can be removed by vacuum drying, etc.

For some dosage forms, particularly the dosage forms having the compositions represented in Table 2, a cyclodextrin (e.g., SBEβCD) may be employed in a molar ratio to the API that is about 1, such as about 0.5-1.5 or about 0.8-1.2, or about 1-2, such as about 1-1.5 or about 1.5-2.

For some dosage forms, a cyclodextrin (e.g., SBEβCD) may be employed in a weight ratio to the API, such as an API in Table 2, in the range of about 1-1000 (e.g. 1 g of cyclodextrin per 1 g of API is a weight ratio of 1); about 1-500, about 1-100; about 1-50; about 1-20; about 1-10; about 1-15; about 1-5, about 2-4, about 3-5, about 4-6, about 5-7, about 6-8, about 7-9, about 8-10, about 9-11, about 10-12, about 11-13, about 12-14, about 13-15, about 14-16, about 15-17, about 16-18, about 17-19, about 18-20, about 19-21, about 0.01-1; about 0.05-1; about 0.1-1; about 0.2-1; about 0.3-1, about 0.4-1, about 0.5-1, about 0.6-1, about 0.7-1, about 0.8-1, or any weight ratio in a range bounded by, or between, any of these values. Each type of cyclodextrin employed may have a different weight ratio to the API in the dosage form.

For some dosage forms, the cyclodextrin (such as SBEβCD) may be present in an amount of about 1-200 mg; about 1-100 mg; 25-175 mg; about 50-150 mg; about 50-100 mg; about 50-200 mg; about 25-100 mg; about 75-150 mg; about 100-175 mg; about 20-80 mg; about 25-50 mg; about 60-100 mg; about 80-100 mg; about 100 mg; about 80-120 mg; about 100-120 mg; about 100-140 mg; about 120-160 mg; about 140-180 mg; about 150-200 mg, about 100-150 mg; about 30-90 mg; about 40-60 mg; about 40-80 mg; about 50-70 mg, about 55-65 mg, about 60-62 mg, about 1-1000 mg, about 500-1000 mg, about 100-500 mg, about 1-50 mg, about 50-75 mg, about 200-250 mg, about 250-300 mg, about 300-500 mg, about 300-400 mg, about 400-500 mg, about 500-600 mg, about 600-700 mg, about 700-1000 mg, about 700-800 mg, about 800-900 mg, about 900-1000 mg, or any amount in a range bounded by, or between, any of these values.

For some dosage forms, an inclusion complex of a drug (such as an API in Table 2) and cyclodextrin is about 1-10%, 5-20%, 5-15%, 6-16%, 7-17%, 8-18%, 9-19%, 10-20%, 15-30%, 30-40%, 40-50%, 50-70%, or 70-90% of the total weight of the dosage form, or any percentage in a range bounded by any of these values.

Some dosage forms contain a bicarbonate (e.g., sodium bicarbonate, or potassium bicarbonate) in amount of about 1-2000 mg; about 1-1000 mg; about 100-1000 mg; about 400-1000 mg, about 200-800 mg; about 1-500 mg; about 1-200 mg; about 1-100 mg; about 50-750 mg; about 500-1000 mg; about 100-500 mg; about 100-300 mg; about 500-1000 mg; about 300-700 mg; about 400-600 mg; about 500-700 mg; up to about 1000 mg; about 50-250 mg; about 50-100 mg; about 250-750 mg; about 100-200 mg; about 200-300 mg; about 300-400 mg; about 400-500 mg; about 410-510 mg; about 420-520 mg; about 430-530 mg; about 440-540 mg; about 450-550 mg; about 460-560 mg; about 470-570 mg; about 480-580 mg; about 490-590 mg; about 500-600 mg; about 600-700 mg; about 700-800 mg; about 800-900 mg; about 900-1000 mg; about 150-650 mg; about 350-850 mg; about 400 mg; about 450 mg; about 500 mg, about 550 mg; about 600 mg; or any amount in a range bounded by, or between, any of these values.

A bicarbonate, such as sodium bicarbonate, may be at least about 10%, at least about 15%, at least about 20%, about 20-40%, about 30-50%, about 40-60%, about 50-70%, about 60-80%, or about 70-90%, or any percentage in a range bounded by any of these values, of the total weight of the dosage form.

In certain embodiments, administering the pharmaceutical composition comprising a drug results in improvement of pharmacokinetics, for example in fasted human subjects, such as increased bioavailability (e.g., reduced Tₘₐₓ, increased Cₘₐₓ, increased AUC, etc.) of the drug, such as an API of Table 2, in the dosage form as compared to a reference dosage form containing the drug but not containing a cyclodextrin and/or a bicarbonate. In some embodiments, the bioavailability of the drug will increase with repeated dosing. For example, the bioavailability of the drug (such as an API of Table 2) in the dosage form, for example in fasted human subjects, may increase after about 1-10 days of repeated dosing; about 2-6 days of repeated dosing; about 3-5 days of repeated dosing; about 4-6 days of repeated dosing; about 5-8 days of repeated dosing; about 5 days of repeated dosing; about 6 days of repeated dosing; about 7 days of repeated dosing; about 8 days of repeated dosing; about 10 days of repeated dosing; about 15 days of repeated dosing; or time period in any range bounded by, or between, any of these values; as compared to the bioavailability of the drug in a dosage form not containing a cyclodextrin and/or a bicarbonate. Orally administering a drug in any dosage forms described herein to a human subject or patient may improve or achieve the desired oral pharmacokinetic properties of the drug.

Any reference to Tₘₐₓ, Cₘₐₓ, AUC, or any other pharmacokinetic parameter should be understood to include an average, mean, or median value in human beings, such as human patients or human subjects.

Unless otherwise indicated, the AUC refers to the AUC calculated to the last measured concentration (AUC₀₋ₜ), such as over a period of 12 hours (AUC₀₋₁₂), over a period of 24 hours (AUC₀₋₂₄) or extrapolated to infinity (AUC_{0-inf}).

For example, a method described herein may reduce the Tₘₐₓ of the API, such as a median, mean, or average Tₘₐₓ of an API of Table 2 in human beings. In some embodiments, the method may include treating a patient to achieve the Tₘₐₓ of an API of Table 2 in the patient within about 10 minutes; within about 20 minutes; within about 30 minutes; within about 40 minutes; within about 50 minutes; within about 60 minutes; within about 70 minutes; within about 80 minutes; within about 90 minutes; within about 100 minutes; within about 110 minutes; within about 120 minutes; within about 180 minutes; about 10-30 minutes; about 20-40 minutes, about 30-50 minutes, about 40-60 minutes; about 50-70 minutes; about 60-90 minutes; about 70-100 minutes; about 80-110 minutes; about 90-120 minutes; about 1-10 hr; about 2-9 hr; about 3-7 hr; about 4-6 hr; about 1-5 hr; about 2-7 hr; about 3-8 hr; about 4-9 hr; about 1-4 hr; about 2-5 hr; about 3-6 hr; about 4-7 hr; about 5-8 hr; about 6-9 hr; about 7-10 hr; or any Tₘₐₓ in a range bounded by, or between, any of these values; after administration of the dosage forms described above.

In some embodiments, an oral dosage form may have a time to half-maximal plasma concentration of an API, such as a median, mean, or average time to half-maximal plasma concentration in human beings, that is less than about 5 minutes; less than about 10 minutes; less than about 15 minutes; less than about 20 minutes; less than about 25 minutes; less than about 30 minutes; less than about 35 minutes; less than about 40 minutes; less than about 45 minutes; less than about 50 minutes; less than about 55 minutes; less than about 60 minutes; less than about 90 minutes; about 5-15 minutes; about 10-20 minutes, about 15-25 minutes, about 20-30 minutes; about 25-35 minutes; about 30-45 minutes; about 35-50 minutes; about 40-55 minutes; about 45-60 minutes; about 0.5-5 hours; or any time in a range bounded by any of these values.

In some embodiments, the dosage form may be formulated for oral administration, for example, with an inert diluent or with an edible carrier, or it may be enclosed in hard or soft shell gelatin capsules, compressed into tablets, or incorporated directly with the food of the diet. For oral therapeutic administration, the active compound may be incorporated with an excipient and used in the form of ingestible tablets, buccal tablets, coated tablets, troches, capsules, elixirs, dispersions, suspensions, solutions, syrups, wafers, patches, and the like.

Tablets, troches, pills, capsules and the like may also contain one or more of the following: a binder such as gum tragacanth, acacia, corn starch or gelatin; an excipient, such as dicalcium phosphate; a disintegrating agent such as corn starch, potato starch, alginic acid and the like; a lubricant such as magnesium stearate; a sweetening agent such as sucrose, lactose or saccharin; or a flavoring agent such as peppermint, oil of wintergreen or cherry flavoring. When the unit dosage form is a capsule, it may contain, in addition to materials of the above type, a liquid carrier. Various other materials may be present as coating, for instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. It may be desirable for material in a dosage form or pharmaceutical composition to be pharmaceutically pure and substantially non-toxic in the amounts employed.

Some compositions or dosage forms may be a liquid or may comprise a solid phase dispersed in a liquid.

The dosage form may further comprise an additional therapeutically active agents, such as an acid inhibitor or an analgesic.

In some embodiments, the dosage form, such as a dosage form containing the compositions indicated in Table 2, may further comprise an acid inhibitor present in an amount effective to raise the gastric pH of a patient to at least 2, to at least 2.5, to at least 3, to at least 3.5, to at least 4, or at least 5, when one or more units of dosage forms are administered. The term “acid inhibitor” refers to agents that inhibit gastric acid secretion and increase gastric pH. Specific H₂ blockers, also referred to as H₂ antagonists or histamine H₂ blockers or antagonists, which may be used include but are not limited to cimetidine, ranitidine, ebrotidine, pabutidine, lafutidine, loxtidine, famotidine, or combinations thereof.

Other agents that may be effectively used as acid inhibitors are the proton pump inhibitors such as omeprazole, esomeprazole, pantoprazole, lansoprazole, dexlansoprazole, rabeprazole, pariprazole, leminoprazole and tenatoprazole. In some embodiments the daily dose of the acid inhibitor, such as esomeprazole, is about 1-200 mg, about 1-100 mg, about 50-100 mg, about 1-50 mg, about 40-80 mg, about 5-50 mg, about 20-40 mg, about 10-50 mg, about 10-20 mg, about 20-40 mg, about 15-50 mg, about 30-60 mg, about 10 mg, about 20 mg, about 30 mg, about 40 mg or any other amount in a range bounded by, or between, any of these values.

Examples of particular proton pump inhibitors include esomeprazole, present in unit dosage forms in an amount of between 5 mg and 50 mg; omeprazole, present in unit dosage forms in an amount of between 5 mg and 50 mg; lansoprazole, present in unit dosage forms in an amount of between 5 mg and 150 mg (and preferably at between 5 mg and 30 mg); and pantoprazole, present in unit dosage forms in an amount of between 10 mg and 200 mg. In some embodiments, the proton pump inhibitor (such as esomeprazole) is present in the dosage form in an amount of about 10-30 mg, about 20-40 mg, about 30-50 mg, about 40-60 mg, about 50-70 mg, about 60-80 mg, about 70-90 mg, or about 80-100 mg. Recently, a newer class of acid inhibitor has been developed which competes with potassium at the acid pump. The compounds in this class have been referred to as “reversible proton pump inhibitors” or “acid pump antagonists” and may also be used. Examples include AZD-0865, AR-H047108, CS-526, pumaprazole, revaprazan and soraprazan (see WO9605177 and WO9605199). Other compounds in this group are H-335/25 (AstraZeneca, Dialog file 128, accession number 020806); Sch-28080 (Schering Plough, Dialog file 128, accession number 009663); Sch-32651 (Schering Plough, Dialog file 128, accession number 006883) and SK&F-96067 (CAS Registry no. 115607-61-9).

In other embodiments, the dosage form or treatment may further comprise administering an opioid in an amount effective to reduce or eliminate pain or inflammation. The opioid may include, but is not limited to, (dextro) propoxyphene, A-methylfentanyl, alfentanil, allylprodine, bezitramide, buprenorphine, butorphanol, carfentanyl, desmethylprodine, dextromoramide, dezocine, diacetylmorphine, dihydrocodeinone, dihydroetorphine, dimorphone, diphenoxylate, dipipanone, etorphine, fentanyl, ketobemidone, lefetamine, levacetylmethadol, levomethorphan, levorphanol, loperamide, meperidine, meptazinol, methadone, methylmorphine, morphine, nalbuphine, nalmefene, naloxone, naltrexone, nicomorphine, ohmefentanyl, oripavine, oxycodone, oxymorphone, PEPAP, paramorphine, pentazocine, phenazocine, piritramide, prodine, remifentanil, sufentanil, tapentadol, tilidine, tramadol, or combinations thereof.

Dosage forms having formulas F1-F100, depicted in Table 1 below, are of particular interest. F1-F100 may further contain other ingredients, such as a binder (e.g. 0.1-10 mg or 0.1-100 mg), an excipient (e.g. 0.1-10 mg or 0.1-100 mg), a disintegrating agent (e.g. 0.1-10 mg or 0.1-100 mg), a lubricant (e.g. 0.1-10 mg or 0.1-100 mg), or other additives.

TABLE 1

| | Amount of | | Amount of | |
|---|---|---|---|---|
| | Bicarbonate, such | | cyclodextrin, such | |
| Formula | as NaHCO₃ | | as SBEβCD | |
| F1 | 0-100 | mg | 0-50 | mg |
| F2 | 100-200 | mg | 0-50 | mg |
| F3 | 200-300 | mg | 0-50 | mg |
| F4 | 300-400 | mg | 0-50 | mg |
| F5 | 400-500 | mg | 0-50 | mg |
| F6 | 500-600 | mg | 0-50 | mg |
| F7 | 600-700 | mg | 0-50 | mg |
| F8 | 700-800 | mg | 0-50 | mg |
| F9 | 800-900 | mg | 0-50 | mg |
| F10 | 900-1000 | mg | 0-50 | mg |
| F11 | 0-100 | mg | 50-100 | mg |
| F12 | 100-200 | mg | 50-100 | mg |
| F13 | 200-300 | mg | 50-100 | mg |
| F14 | 300-400 | mg | 50-100 | mg |
| F15 | 400-500 | mg | 50-100 | mg |
| F16 | 500-600 | mg | 50-100 | mg |
| F17 | 600-700 | mg | 50-100 | mg |
| F18 | 700-800 | mg | 50-100 | mg |
| F19 | 800-900 | mg | 50-100 | mg |
| F20 | 900-1000 | mg | 50-100 | mg |
| F21 | 0-100 | mg | 100-150 | mg |
| F22 | 100-200 | mg | 100-150 | mg |
| F23 | 200-300 | mg | 100-150 | mg |
| F24 | 300-400 | mg | 100-150 | mg |
| F25 | 400-500 | mg | 100-150 | mg |
| F26 | 500-600 | mg | 100-150 | mg |
| F27 | 600-700 | mg | 100-150 | mg |
| F28 | 700-800 | mg | 100-150 | mg |
| F29 | 800-900 | mg | 100-150 | mg |
| F30 | 900-1000 | mg | 100-150 | mg |
| F31 | 0-100 | mg | 150-200 | mg |
| F32 | 100-200 | mg | 150-200 | mg |
| F33 | 200-300 | mg | 150-200 | mg |
| F34 | 300-400 | mg | 150-200 | mg |
| F35 | 400-500 | mg | 150-200 | mg |
| F36 | 500-600 | mg | 150-200 | mg |
| F37 | 600-700 | mg | 150-200 | mg |
| F38 | 700-800 | mg | 150-200 | mg |
| F39 | 800-900 | mg | 150-200 | mg |
| F40 | 900-1000 | mg | 150-200 | mg |
| F41 | 0-100 | mg | 200-250 | mg |
| F42 | 100-200 | mg | 200-250 | mg |
| F43 | 200-300 | mg | 200-250 | mg |
| F44 | 300-400 | mg | 200-250 | mg |
| F45 | 400-500 | mg | 200-250 | mg |
| F46 | 500-600 | mg | 200-250 | mg |
| F47 | 600-700 | mg | 200-250 | mg |
| F48 | 700-800 | mg | 200-250 | mg |
| F49 | 800-900 | mg | 200-250 | mg |
| F50 | 900-1000 | mg | 200-250 | mg |
| F51 | 0-100 | mg | 250-300 | mg |
| F52 | 100-200 | mg | 250-300 | mg |
| F53 | 200-300 | mg | 250-300 | mg |
| F54 | 300-400 | mg | 250-300 | mg |
| F55 | 400-500 | mg | 250-300 | mg |
| F56 | 500-600 | mg | 250-300 | mg |
| F57 | 600-700 | mg | 250-300 | mg |
| F58 | 700-800 | mg | 250-300 | mg |
| F59 | 800-900 | mg | 250-300 | mg |
| F60 | 900-1000 | mg | 250-300 | mg |
| F61 | 0-100 | mg | 300-500 | mg |
| F62 | 100-200 | mg | 300-500 | mg |
| F63 | 200-300 | mg | 300-500 | mg |
| F64 | 300-400 | mg | 300-500 | mg |
| F65 | 400-500 | mg | 300-500 | mg |
| F66 | 500-600 | mg | 300-500 | mg |
| F67 | 600-700 | mg | 300-500 | mg |
| F68 | 700-800 | mg | 300-500 | mg |
| F69 | 800-900 | mg | 300-500 | mg |
| F70 | 900-1000 | mg | 300-500 | mg |
| F71 | 0-100 | mg | 500-600 | mg |
| F72 | 100-200 | mg | 500-600 | mg |
| F73 | 200-300 | mg | 500-600 | mg |
| F74 | 300-400 | mg | 500-600 | mg |
| F75 | 400-500 | mg | 500-600 | mg |
| F76 | 500-600 | mg | 500-600 | mg |
| F77 | 600-700 | mg | 500-600 | mg |
| F78 | 700-800 | mg | 500-600 | mg |
| F79 | 800-900 | mg | 500-600 | mg |
| F80 | 900-1000 | mg | 500-600 | mg |
| F81 | 0-100 | mg | 600-700 | mg |
| F82 | 100-200 | mg | 600-700 | mg |
| F83 | 200-300 | mg | 600-700 | mg |
| F84 | 300-400 | mg | 600-700 | mg |
| F85 | 400-500 | mg | 600-700 | mg |
| F86 | 500-600 | mg | 600-700 | mg |
| F87 | 600-700 | mg | 600-700 | mg |
| F88 | 700-800 | mg | 600-700 | mg |
| F89 | 800-900 | mg | 600-700 | mg |
| F90 | 900-1000 | mg | 600-700 | mg |
| F91 | 0-100 | mg | 700-1000 | mg |
| F92 | 100-200 | mg | 700-1000 | mg |
| F93 | 200-300 | mg | 700-1000 | mg |
| F94 | 300-400 | mg | 700-1000 | mg |
| F95 | 400-500 | mg | 700-1000 | mg |
| F96 | 500-600 | mg | 700-1000 | mg |
| F97 | 600-700 | mg | 700-1000 | mg |
| F98 | 700-800 | mg | 700-1000 | mg |
| F99 | 800-900 | mg | 700-1000 | mg |
| F100 | 900-1000 | mg | 700-1000 | mg |

Active pharmaceutical ingredients (API) that may be used include any listed in Table 2 below. In particular, each API may be present in one of the formulas identified in Table 2 with the API.

TABLE 2

| Active | |
|---|---|
| Pharma- | |
| ceutical | |
| Agent | Formula of Dosage Form Containing API |
| abacavir | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| abaloparatide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| abemaciclib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| abiraterone | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acalabrutinib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acamprosate | F1, F2, F3, F4, F5, F6, F7, F8, F9, F10, F11, F12, |
| calcium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acarbose | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acebutolol | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acetamino- | F1, F2, F3, F4, F5, F6, F7, F8, F9, F10, F11, F12, |
| phen | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acetazolamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acetazolamide | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acetic acid | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aceto- | F1, F2, F3, F4, F5, F6, F7, F8, F9, F10, F11, F12, |
| hydroxamic | F13, F14, F15, F16, F17, F18, F19, F20, F21, F22, F23, |
| acid | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acetylcholine | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acetylcysteine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acitretin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aclidinium | F1, F2, F3, F4, F5, F6, F7, F8, F9, F10, F11, F12, |
| bromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acrivastine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| acyclovir | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| adapalene | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| adefovir | F1, F2, F3, F4, F5, F6, F7, F8, F9, F10, F11, F12, |
| dipivoxil | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| adenosine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| afatinib | F1, F2, F3, F4, F5, F6, F7, F8, F9, F10, F11, F12, |
| dimaleate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| albendazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| albumin | F1, F2, F3, F4, F5, F6, F7, F8, F9, F10, F11, F12, |
| human | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| albumin | F1, F2, F3, F4, F5, F6, F7, F8, F9, F10, F11, F12, |
| iodinated | F13, F14, F15, F16, F17, F18, F19, F20, F21, F22, F23, |
| I-125 | F24, F25, F26, F27, F28, F29, F30, F31, F32, F33, |
| serum | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| albumin | F1, F2, F3, F4, F5, F6, F7, F8, F9, F10, F11, F12, |
| iodinated | F13, F14, F15, F16, F17, F18, F19, F20, F21, F22, F23, |
| I-131 | F24, F25, F26, F27, F28, F29, F30, F31, F32, F33, |
| serum | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| albuterol | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alcaftadine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alclometasone | F1, F2, F3, F4, F5, F6, F7, F8, F9, F10, F11, F12, |
| dipropionate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alectinib | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alendronate | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alfentanil | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alfuzosin | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aliskiren | F1, F2, F3, F4, F5, F6, F7, F8, F9, F10, F11, F12, |
| hemifumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alitretinoin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| allopurinol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| allopurinol | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| almotriptan | F1, F2, F3, F4, F5, F6, F7, F8, F9, F10, F11, F12, |
| malate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alogliptin | F1, F2, F3, F4, F5, F6, F7, F8, F9, F10, F11, F12, |
| benzoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alosetron | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alpha- | F1, F2, F3, F4, F5, F6, F7, F8, F9, F10, F11, F12, |
| tocopherol | F13, F14, F15, F16, F17, F18, F19, F20, F21, F22, F23, |
| acetate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alprazolam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alprostadil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| altretamine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aluminum | F1, F2, F3, F4, F5, F6, F7, F8, F9, F10, F11, F12, |
| hydroxide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| alvimopan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amantadine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ambrisentan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amcinonide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amifostine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amikacin | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amiloride | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amino acids | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aminocaproic | F1, F2, F3, F4, F5, F6, F7, F8, F9, F10, F11, F12, |
| acid | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aminolevulinic | F1, F2, F3, F4, F5, F6, F7, F8, F9, F10, F11, F12, |
| acid | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aminophylline | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aminosalicylic | F1, F2, F3, F4, F5, F6, F7, F8, F9, F10, F11, F12, |
| acid | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amiodarone | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amitriptyline | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amlodipine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydrochloro- | F1, F2, F3, F4, F5, F6, F7, F8, F9, F10, F11, F12, |
| thiazide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amlodipine | F1, F2, F3, F4, F5, F6, F7, F8, F9, F10, F11, F12, |
| besylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ammonium | F1, F2, F3, F4, F5, F6, F7, F8, F9, F10, F11, F12, |
| lactate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amoxapine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amoxicillin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clarithromycin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amp aspartate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amphetamine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amphetamine | F1, F2, F3, F4, F5, F6, F7, F8, F9, F10, F11, F12, |
| aspartate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amphetamine | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| amphotericin | F1, F2, F3, F4, F5, F6, F7, F8, F9, F10, F11, F12, |
| b | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ampicillin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ampicillin/ | F1, F2, F3, F4, F5, F6, F7, F8, F9, F10, F11, F12, |
| ampicillin | F13, F14, F15, F16, F17, F18, F19, F20, F21, F22, F23, |
| trihydrate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| anagrelide | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| anastrozole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| magnesium | F1, F2, F3, F4, F5, F6, F7, F8, F9, F10, F11, F12, |
| hydroxide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| angiotensin ii | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| anidulafungin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| apalutamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| apixaban | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| apomorphine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| apraclonidine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| apremilast | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aprepitant | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| arformoterol | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| argatroban | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| arginine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aripiprazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aripiprazole | F1, F2, F3, F4, F5, F6, F7, F8, F9, F10, F11, F12, |
| lauroxil | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| armodafinil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| arsenic | F1, F2, F3, F4, F5, F6, F7, F8, F9, F10, F11, F12, |
| trioxide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| artemether | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| articaine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ascorbic acid | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| asenapine | F1, F2, F3, F4, F5, F6, F7, F8, F9, F10, F11, F12, |
| maleate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aspirin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| codeine | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| atazanavir | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| atenolol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| atomoxetine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| atorvastatin | F1, F2, F3, F4, F5, F6, F7, F8, F9, F10, F11, F12, |
| calcium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| atovaquone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| atracurium | F1, F2, F3, F4, F5, F6, F7, F8, F9, F10, F11, F12, |
| besylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| atropine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| atropine | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| auranofin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| avanafil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| avatrombopag | F1, F2, F3, F4, F5, F6, F7, F8, F9, F10, F11, F12, |
| maleate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| avibactam | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| avobenzone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| axitinib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| azacitidine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| azathioprine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| azathioprine | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| azelaic acid | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| azelastine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| azilsartan | F1, F2, F3, F4, F5, F6, F7, F8, F9, F10, F11, F12, |
| kamedoxomil | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| azithromycin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| aztreonam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bacitracin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bacitracin zinc | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bacitracin | F1, F2, F3, F4, F5, F6, F7, F8, F9, F10, F11, F12, |
| zinc and | F13, F14, F15, F16, F17, F18, F19, F20, F21, F22, F23, |
| hydrocortisone | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| baclofen | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| balsalazide | F1, F2, F3, F4, F5, F6, F7, F8, F9, F10, F11, F12, |
| disodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| baricitinib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| barium sulfate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bazedoxifene | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| beclo- | F1, F2, F3, F4, F5, F6, F7, F8, F9, F10, F11, F12, |
| methasone | F13, F14, F15, F16, F17, F18, F19, F20, F21, F22, F23, |
| dipropionate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| beclo- | F1, F2, F3, F4, F5, F6, F7, F8, F9, F10, F11, F12, |
| methasone | F13, F14, F15, F16, F17, F18, F19, F20, F21, F22, F23, |
| dipropionate | F24, F25, F26, F27, F28, F29, F30, F31, F32, F33, |
| monohydrate | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bedaquiline | F1, F2, F3, F4, F5, F6, F7, F8, F9, F10, F11, F12, |
| fumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| belinostat | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| benazepril | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bendamustine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bendro- | F1, F2, F3, F4, F5, F6, F7, F8, F9, F10, F11, F12, |
| flumethiazide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| benoxinate | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bentoquatam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| benznidazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| benzonatate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| benzoyl | F1, F2, F3, F4, F5, F6, F7, F8, F9, F10, F11, F12, |
| peroxide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| benz- | F1, F2, F3, F4, F5, F6, F7, F8, F9, F10, F11, F12, |
| phetamine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| benztropine | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| benzyl | F1, F2, F3, F4, F5, F6, F7, F8, F9, F10, F11, F12, |
| alcohol | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| benzyl- | F1, F2, F3, F4, F5, F6, F7, F8, F9, F10, F11, F12, |
| penicilloyl | F13, F14, F15, F16, F17, F18, F19, F20, F21, F22, F23, |
| polylysine | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bepotastine | F1, F2, F3, F4, F5, F6, F7, F8, F9, F10, F11, F12, |
| besilate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| beractant | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| besifloxacin | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| betaine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| beta- | F1, F2, F3, F4, F5, F6, F7, F8, F9, F10, F11, F12, |
| methasone | F13, F14, F15, F16, F17, F18, F19, F20, F21, F22, F23, |
| acetate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| beta- | F1, F2, F3, F4, F5, F6, F7, F8, F9, F10, F11, F12, |
| methasone | F13, F14, F15, F16, F17, F18, F19, F20, F21, F22, F23, |
| dipropionate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| beta- | F1, F2, F3, F4, F5, F6, F7, F8, F9, F10, F11, F12, |
| methasone | F13, F14, F15, F16, F17, F18, F19, F20, F21, F22, F23, |
| sodium | F24, F25, F26, F27, F28, F29, F30, F31, F32, F33, |
| phosphate | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| beta- | F1, F2, F3, F4, F5, F6, F7, F8, F9, F10, F11, F12, |
| methasone | F13, F14, F15, F16, F17, F18, F19, F20, F21, F22, F23, |
| valerate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bethanechol | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| betrixaban | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bexarotene | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bicalutamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bictegravir | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bimatoprost | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| binimetinib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bismuth | F1, F2, F3, F4, F5, F6, F7, F8, F9, F10, F11, F12, |
| subcitrate | F13, F14, F15, F16, F17, F18, F19, F20, F21, F22, F23, |
| potassium | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bisoprolol | F1, F2, F3, F4, F5, F6, F7, F8, F9, F10, F11, F12, |
| fumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bivalirudin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bleomycin | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bortezomib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bosentan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bosutinib | F1, F2, F3, F4, F5, F6, F7, F8, F9, F10, F11, F12, |
| monohydrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| brexpiprazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| brigatinib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| brimonidine | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| brimonidine | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| brinzolamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| brivaracetam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bromfenac | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bromocriptine | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bromph- | F1, F2, F3, F4, F5, F6, F7, F8, F9, F10, F11, F12, |
| eniramine | F13, F14, F15, F16, F17, F18, F19, F20, F21, F22, F23, |
| maleate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| budesonide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| budesonide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bumetanide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bupivacaine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bupivacaine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bupre- | F1, F2, F3, F4, F5, F6, F7, F8, F9, F10, F11, F12, |
| norphine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bupre- | F1, F2, F3, F4, F5, F6, F7, F8, F9, F10, F11, F12, |
| norphine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bupropion | F1, F2, F3, F4, F5, F6, F7, F8, F9, F10, F11, F12, |
| hydrobromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| bupropion | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| buspirone | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| busulfan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| butabarbital | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| butenafine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| butenafine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| butoconazole | F1, F2, F3, F4, F5, F6, F7, F8, F9, F10, F11, F12, |
| nitrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| butorphanol | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cabazitaxel | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cabergoline | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cabozantinib | F1, F2, F3, F4, F5, F6, F7, F8, F9, F10, F11, F12, |
| s-malate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| caffeine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dihy- | F1, F2, F3, F4, F5, F6, F7, F8, F9, F10, F11, F12, |
| drocodeine | F13, F14, F15, F16, F17, F18, F19, F20, F21, F22, F23, |
| bitartrate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| caffeine | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| calcifediol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| calcipotriene | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| calcitonin | F1, F2, F3, F4, F5, F6, F7, F8, F9, F10, F11, F12, |
| salmon | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| calcitriol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| calcium | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| calcium | F1, F2, F3, F4, F5, F6, F7, F8, F9, F10, F11, F12, |
| carbonate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| calcium | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| calcium | F1, F2, F3, F4, F5, F6, F7, F8, F9, F10, F11, F12, |
| gluconate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| calfactant | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| canagliflozin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| candesartan | F1, F2, F3, F4, F5, F6, F7, F8, F9, F10, F11, F12, |
| cilexetil | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cangrelor | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| capecitabine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| capreomycin | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| capsaicin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| captopril | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carbachol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carba- | F1, F2, F3, F4, F5, F6, F7, F8, F9, F10, F11, F12, |
| mazepine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carbidopa | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carbin- | F1, F2, F3, F4, F5, F6, F7, F8, F9, F10, F11, F12, |
| oxamine | F13, F14, F15, F16, F17, F18, F19, F20, F21, F22, F23, |
| maleate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carboplatin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carboprost | F1, F2, F3, F4, F5, F6, F7, F8, F9, F10, F11, F12, |
| trom- | F13, F14, F15, F16, F17, F18, F19, F20, F21, F22, F23, |
| ethamine | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carfilzomib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carglumic | F1, F2, F3, F4, F5, F6, F7, F8, F9, F10, F11, F12, |
| acid | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cariprazine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carisoprodol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carmustine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carteolol | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carvedilol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| carvedilol | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| caspofungin | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefaclor | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefadroxil/ | F1, F2, F3, F4, F5, F6, F7, F8, F9, F10, F11, F12, |
| cefadroxil | F13, F14, F15, F16, F17, F18, F19, F20, F21, F22, F23, |
| hemihydrate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefazolin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefdinir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefepime | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefixime | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefotaxime | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefotetan | F1, F2, F3, F4, F5, F6, F7, F8, F9, F10, F11, F12, |
| disodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefoxitin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefpodoxime | F1, F2, F3, F4, F5, F6, F7, F8, F9, F10, F11, F12, |
| proxetil | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefprozil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ceftaroline | F1, F2, F3, F4, F5, F6, F7, F8, F9, F10, F11, F12, |
| fosamil | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ceftazidime | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ceftolozane | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ceftriaxone | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefuroxime | F1, F2, F3, F4, F5, F6, F7, F8, F9, F10, F11, F12, |
| axetil | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cefuroxime | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| celecoxib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cephalexin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ceritinib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cetirizine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cetrorelix | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cevimeline | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chenodiol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlorambucil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chloram- | F1, F2, F3, F4, F5, F6, F7, F8, F9, F10, F11, F12, |
| phenicol | F13, F14, F15, F16, F17, F18, F19, F20, F21, F22, F23, |
| sodium | F24, F25, F26, F27, F28, F29, F30, F31, F32, F33, |
| succinate | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlor- | F1, F2, F3, F4, F5, F6, F7, F8, F9, F10, F11, F12, |
| diazepoxide | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlorhexidine | F1, F2, F3, F4, F5, F6, F7, F8, F9, F10, F11, F12, |
| gluconate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chloro- | F1, F2, F3, F4, F5, F6, F7, F8, F9, F10, F11, F12, |
| procaine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chloroquine | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlorothiazide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlorothiazide | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlor- | F1, F2, F3, F4, F5, F6, F7, F8, F9, F10, F11, F12, |
| pheniramine | F13, F14, F15, F16, F17, F18, F19, F20, F21, F22, F23, |
| maleate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlor- | F1, F2, F3, F4, F5, F6, F7, F8, F9, F10, F11, F12, |
| pheniramine | F13, F14, F15, F16, F17, F18, F19, F20, F21, F22, F23, |
| maleate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pseudo- | F1, F2, F3, F4, F5, F6, F7, F8, F9, F10, F11, F12, |
| ephedrine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlor- | F1, F2, F3, F4, F5, F6, F7, F8, F9, F10, F11, F12, |
| pheniramine | F13, F14, F15, F16, F17, F18, F19, F20, F21, F22, F23, |
| polistirex | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlor- | F1, F2, F3, F4, F5, F6, F7, F8, F9, F10, F11, F12, |
| promazine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlor- | F1, F2, F3, F4, F5, F6, F7, F8, F9, F10, F11, F12, |
| propamide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlorthalidone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chlorzoxazone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| choles- | F1, F2, F3, F4, F5, F6, F7, F8, F9, F10, F11, F12, |
| tyramine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cholic acid | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| choline c-11 | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| choline | F1, F2, F3, F4, F5, F6, F7, F8, F9, F10, F11, F12, |
| fenofibrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chorio- | F1, F2, F3, F4, F5, F6, F7, F8, F9, F10, F11, F12, |
| gonadotropin | F13, F14, F15, F16, F17, F18, F19, F20, F21, F22, F23, |
| alfa | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chorionic | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| chromic | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ciclesonide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ciclopirox | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cidofovir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cilastatin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cilostazol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cimetidine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cimetidine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cinacalcet | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ciprofloxacin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ciprofloxacin | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cisatracurium | F1, F2, F3, F4, F5, F6, F7, F8, F9, F10, F11, F12, |
| besylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cisplatin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| citalopram | F1, F2, F3, F4, F5, F6, F7, F8, F9, F10, F11, F12, |
| hydrobromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| citric acid | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cladribine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clarithro- | F1, F2, F3, F4, F5, F6, F7, F8, F9, F10, F11, F12, |
| mycin | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clemastine | F1, F2, F3, F4, F5, F6, F7, F8, F9, F10, F11, F12, |
| fumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clevidipine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clindamycin | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clindamycin | F1, F2, F3, F4, F5, F6, F7, F8, F9, F10, F11, F12, |
| palmitate | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clindamycin | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clobazam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clobetasol | F1, F2, F3, F4, F5, F6, F7, F8, F9, F10, F11, F12, |
| propionate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clocortolone | F1, F2, F3, F4, F5, F6, F7, F8, F9, F10, F11, F12, |
| pivalate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clofarabine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clomiphene | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clomipramine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clonazepam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clonidine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clonidine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clopidogrel | F1, F2, F3, F4, F5, F6, F7, F8, F9, F10, F11, F12, |
| bisulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clorazepate | F1, F2, F3, F4, F5, F6, F7, F8, F9, F10, F11, F12, |
| dipotassium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clotrimazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| clozapine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cobicistat | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cobimetinib | F1, F2, F3, F4, F5, F6, F7, F8, F9, F10, F11, F12, |
| fumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cocaine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| codeine | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| codeine | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| colchicine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| colesevelam | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| colestipol | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| colistimethate | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| colistin | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| conivaptan | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| copanlisib | F1, F2, F3, F4, F5, F6, F7, F8, F9, F10, F11, F12, |
| dihydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| corticorelin | F1, F2, F3, F4, F5, F6, F7, F8, F9, F10, F11, F12, |
| ovine | F13, F14, F15, F16, F17, F18, F19, F20, F21, F22, F23, |
| triflutate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| corticotropin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cortisone | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cosyntropin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| crisaborole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| crizotinib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| crofelemer | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cromolyn | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| crotamiton | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cupric | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cyano- | F1, F2, F3, F4, F5, F6, F7, F8, F9, F10, F11, F12, |
| cobalamin | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cyclo- | F1, F2, F3, F4, F5, F6, F7, F8, F9, F10, F11, F12, |
| benzaprine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cyclopentolate | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cyclo- | F1, F2, F3, F4, F5, F6, F7, F8, F9, F10, F11, F12, |
| phosphamide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cycloserine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cyclosporine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cypro- | F1, F2, F3, F4, F5, F6, F7, F8, F9, F10, F11, F12, |
| heptadine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cysteamine | F1, F2, F3, F4, F5, F6, F7, F8, F9, F10, F11, F12, |
| bitartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cysteamine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| cytarabine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dabigatran | F1, F2, F3, F4, F5, F6, F7, F8, F9, F10, F11, F12, |
| etexilate | F13, F14, F15, F16, F17, F18, F19, F20, F21, F22, F23, |
| mesylate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dabrafenib | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dacarbazine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| daclatasvir | F1, F2, F3, F4, F5, F6, F7, F8, F9, F10, F11, F12, |
| dihy- | F13, F14, F15, F16, F17, F18, F19, F20, F21, F22, F23, |
| drochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dactinomycin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dalbavancin | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dalfampridine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dalfopristin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dalteparin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| danazol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dantrolene | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dapagliflozin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dapsone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| daptomycin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| darifenacin | F1, F2, F3, F4, F5, F6, F7, F8, F9, F10, F11, F12, |
| hydrobromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| darunavir | F1, F2, F3, F4, F5, F6, F7, F8, F9, F10, F11, F12, |
| ethanolate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dasabuvir | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dasatinib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| daunorubicin | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| decitabine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| deferasirox | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| deferiprone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| deferoxamine | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| defibrotide | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| deflazacort | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| degarelix | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| delafloxacin | F1, F2, F3, F4, F5, F6, F7, F8, F9, F10, F11, F12, |
| meglumine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| delavirdine | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| deme- | F1, F2, F3, F4, F5, F6, F7, F8, F9, F10, F11, F12, |
| clocycline | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| deoxycholic | F1, F2, F3, F4, F5, F6, F7, F8, F9, F10, F11, F12, |
| acid | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| desflurane | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| desipramine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| desirudin | F1, F2, F3, F4, F5, F6, F7, F8, F9, F10, F11, F12, |
| recombinant | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| desloratadine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| desmopressin | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| desogestrel | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| desonide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| desoxi- | F1, F2, F3, F4, F5, F6, F7, F8, F9, F10, F11, F12, |
| metasone | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| desven- | F1, F2, F3, F4, F5, F6, F7, F8, F9, F10, F11, F12, |
| lafaxine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| desven- | F1, F2, F3, F4, F5, F6, F7, F8, F9, F10, F11, F12, |
| lafaxine | F13, F14, F15, F16, F17, F18, F19, F20, F21, F22, F23, |
| succinate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| deutetra- | F1, F2, F3, F4, F5, F6, F7, F8, F9, F10, F11, F12, |
| benazine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dexa- | F1, F2, F3, F4, F5, F6, F7, F8, F9, F10, F11, F12, |
| methasone | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dexa- | F1, F2, F3, F4, F5, F6, F7, F8, F9, F10, F11, F12, |
| methasone | F13, F14, F15, F16, F17, F18, F19, F20, F21, F22, F23, |
| sodium | F24, F25, F26, F27, F28, F29, F30, F31, F32, F33, |
| phosphate | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dexchlor- | F1, F2, F3, F4, F5, F6, F7, F8, F9, F10, F11, F12, |
| pheniramine | F13, F14, F15, F16, F17, F18, F19, F20, F21, F22, F23, |
| maleate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dexlan- | F1, F2, F3, F4, F5, F6, F7, F8, F9, F10, F11, F12, |
| soprazole | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dexme- | F1, F2, F3, F4, F5, F6, F7, F8, F9, F10, F11, F12, |
| detomidine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dexmethyl- | F1, F2, F3, F4, F5, F6, F7, F8, F9, F10, F11, F12, |
| phenidate | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dexrazoxane | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dextro- | F1, F2, F3, F4, F5, F6, F7, F8, F9, F10, F11, F12, |
| amphetamine | F13, F14, F15, F16, F17, F18, F19, F20, F21, F22, F23, |
| sulfate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dextro- | F1, F2, F3, F4, F5, F6, F7, F8, F9, F10, F11, F12, |
| methorphan | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dextro- | F1, F2, F3, F4, F5, F6, F7, F8, F9, F10, F11, F12, |
| methorphan | F13, F14, F15, F16, F17, F18, F19, F20, F21, F22, F23, |
| hydrobromide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dextro- | F1, F2, F3, F4, F5, F6, F7, F8, F9, F10, F11, F12, |
| methorphan | F13, F14, F15, F16, F17, F18, F19, F20, F21, F22, F23, |
| polistirex | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dextrose | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diatrizoate | F1, F2, F3, F4, F5, F6, F7, F8, F9, F10, F11, F12, |
| meglumine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diazepam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diazoxide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dichlor- | F1, F2, F3, F4, F5, F6, F7, F8, F9, F10, F11, F12, |
| phenamide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diclofenac | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diclofenac | F1, F2, F3, F4, F5, F6, F7, F8, F9, F10, F11, F12, |
| epolamine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diclofenac | F1, F2, F3, F4, F5, F6, F7, F8, F9, F10, F11, F12, |
| potassium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diclofenac | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dicloxacillin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dicyclomine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| didanosine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dienogest | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diethyl- | F1, F2, F3, F4, F5, F6, F7, F8, F9, F10, F11, F12, |
| propion | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diflorasone | F1, F2, F3, F4, F5, F6, F7, F8, F9, F10, F11, F12, |
| diacetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diflunisal | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| difluprednate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| digoxin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dihydro- | F1, F2, F3, F4, F5, F6, F7, F8, F9, F10, F11, F12, |
| ergotamine | F13, F14, F15, F16, F17, F18, F19, F20, F21, F22, F23, |
| mesylate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diltiazem | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dimen- | F1, F2, F3, F4, F5, F6, F7, F8, F9, F10, F11, F12, |
| hydrinate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dimercaprol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dimethyl | F1, F2, F3, F4, F5, F6, F7, F8, F9, F10, F11, F12, |
| fumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dimethyl | F1, F2, F3, F4, F5, F6, F7, F8, F9, F10, F11, F12, |
| sulfoxide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dinoprostone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diphen- | F1, F2, F3, F4, F5, F6, F7, F8, F9, F10, F11, F12, |
| hydramine | F13, F14, F15, F16, F17, F18, F19, F20, F21, F22, F23, |
| citrate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| diphen- | F1, F2, F3, F4, F5, F6, F7, F8, F9, F10, F11, F12, |
| hydramine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dipyridamole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| disopyramide | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| disulfiram | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| divalproex | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dobutamine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| docetaxel | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| docosanol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dofetilide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dolutegravir | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| donepezil | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dorzolamide | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| doxapram | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| doxazosin | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| doxepin | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| doxer- | F1, F2, F3, F4, F5, F6, F7, F8, F9, F10, F11, F12, |
| calciferol | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| doxorubicin | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| doxycycline | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| doxycycline | F1, F2, F3, F4, F5, F6, F7, F8, F9, F10, F11, F12, |
| calcium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| doxycycline | F1, F2, F3, F4, F5, F6, F7, F8, F9, F10, F11, F12, |
| hyclate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| doxylamine | F1, F2, F3, F4, F5, F6, F7, F8, F9, F10, F11, F12, |
| succinate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dronabinol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dronedarone | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| droperidol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| drospirenone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| droxidopa | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| duloxetine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dutasteride | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| echothiophate | F1, F2, F3, F4, F5, F6, F7, F8, F9, F10, F11, F12, |
| iodide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| econazole | F1, F2, F3, F4, F5, F6, F7, F8, F9, F10, F11, F12, |
| nitrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| edaravone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| edetate | F1, F2, F3, F4, F5, F6, F7, F8, F9, F10, F11, F12, |
| calcium | F13, F14, F15, F16, F17, F18, F19, F20, F21, F22, F23, |
| disodium | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| edoxaban | F1, F2, F3, F4, F5, F6, F7, F8, F9, F10, F11, F12, |
| tosylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| edrophonium | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| efavirenz | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| efinaconazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| eflornithine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| elbasvir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| eletriptan | F1, F2, F3, F4, F5, F6, F7, F8, F9, F10, F11, F12, |
| hydrobromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| eliglustat | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| eltrombopag | F1, F2, F3, F4, F5, F6, F7, F8, F9, F10, F11, F12, |
| olamine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| eluxadoline | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| emedastine | F1, F2, F3, F4, F5, F6, F7, F8, F9, F10, F11, F12, |
| difumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| empagliflozin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| emtricitabine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| enalapril | F1, F2, F3, F4, F5, F6, F7, F8, F9, F10, F11, F12, |
| maleate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| enalaprilat | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| enasidenib | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| encorafenib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| enfuvirtide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| enoxaparin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| entacapone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| entecavir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| enzalutamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ephedrine | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| epinastine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| epinephrine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| epinephrine | F1, F2, F3, F4, F5, F6, F7, F8, F9, F10, F11, F12, |
| bitartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| epirubicin | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| eplerenone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| epoprostenol | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| eprosartan | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| eptifibatide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ergocalciferol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ergoloid | F1, F2, F3, F4, F5, F6, F7, F8, F9, F10, F11, F12, |
| mesylates | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ergotamine | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| eribulin | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| erlotinib | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ertapenem | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ertugliflozin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| erythromycin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| erythromycin | F1, F2, F3, F4, F5, F6, F7, F8, F9, F10, F11, F12, |
| ethylsuccinate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| erythromycin | F1, F2, F3, F4, F5, F6, F7, F8, F9, F10, F11, F12, |
| lactobionate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| erythromycin | F1, F2, F3, F4, F5, F6, F7, F8, F9, F10, F11, F12, |
| stearate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| escitalopram | F1, F2, F3, F4, F5, F6, F7, F8, F9, F10, F11, F12, |
| oxalate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| esli- | F1, F2, F3, F4, F5, F6, F7, F8, F9, F10, F11, F12, |
| carbazepine | F13, F14, F15, F16, F17, F18, F19, F20, F21, F22, F23, |
| acetate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| esmolol | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| esomeprazole | F1, F2, F3, F4, F5, F6, F7, F8, F9, F10, F11, F12, |
| magnesium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| esomeprazole | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| esomeprazole | F1, F2, F3, F4, F5, F6, F7, F8, F9, F10, F11, F12, |
| strontium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| estazolam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| esterified | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| estradiol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| estradiol | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| estradiol | F1, F2, F3, F4, F5, F6, F7, F8, F9, F10, F11, F12, |
| cypionate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| estradiol | F1, F2, F3, F4, F5, F6, F7, F8, F9, F10, F11, F12, |
| valerate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| estramustine | F1, F2, F3, F4, F5, F6, F7, F8, F9, F10, F11, F12, |
| phosphate | F13, F14, F15, F16, F17, F18, F19, F20, F21, F22, F23, |
| sodium | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| estrogens | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| estropipate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| eszopiclone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| etelcalcetide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| eteplirsen | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ethacrynate | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ethacrynic | F1, F2, F3, F4, F5, F6, F7, F8, F9, F10, F11, F12, |
| acid | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ethambutol | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ethanolamine | F1, F2, F3, F4, F5, F6, F7, F8, F9, F10, F11, F12, |
| oleate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ethinyl | F1, F2, F3, F4, F5, F6, F7, F8, F9, F10, F11, F12, |
| estradiol | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levomefolate | F1, F2, F3, F4, F5, F6, F7, F8, F9, F10, F11, F12, |
| calcium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ethiodized oil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ethionamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ethosuximide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ethotoin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| etidronate | F1, F2, F3, F4, F5, F6, F7, F8, F9, F10, F11, F12, |
| disodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| etodolac | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| etomidate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| etonogestrel | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| etoposide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| etoposide | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| etravirine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| everolimus | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| exemestane | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| exenatide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| exenatide | F1, F2, F3, F4, F5, F6, F7, F8, F9, F10, F11, F12, |
| synthetic | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ezetimibe | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| famciclovir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| famotidine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| febuxostat | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| felbamate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| felodipine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fenofibrate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fenofibric | F1, F2, F3, F4, F5, F6, F7, F8, F9, F10, F11, F12, |
| acid | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fenoldopam | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fenoprofen | F1, F2, F3, F4, F5, F6, F7, F8, F9, F10, F11, F12, |
| calcium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fentanyl | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fentanyl | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ferric | F1, F2, F3, F4, F5, F6, F7, F8, F9, F10, F11, F12, |
| carboxy- | F13, F14, F15, F16, F17, F18, F19, F20, F21, F22, F23, |
| maltose | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ferric citrate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ferric | F1, F2, F3, F4, F5, F6, F7, F8, F9, F10, F11, F12, |
| hexacya- | F13, F14, F15, F16, F17, F18, F19, F20, F21, F22, F23, |
| noferrate | F24, F25, F26, F27, F28, F29, F30, F31, F32, F33, |
| (ii) | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ferric | F1, F2, F3, F4, F5, F6, F7, F8, F9, F10, F11, F12, |
| pyrophosphate | F13, F14, F15, F16, F17, F18, F19, F20, F21, F22, F23, |
| citrate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ferumoxytol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fesoterodine | F1, F2, F3, F4, F5, F6, F7, F8, F9, F10, F11, F12, |
| fumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fexofenadine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fidaxomicin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| finafloxacin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| finasteride | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fingolimod | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fish oil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flavoxate | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flecainide | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flibanserin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| florbetaben | F1, F2, F3, F4, F5, F6, F7, F8, F9, F10, F11, F12, |
| f-18 | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| florbetapir | F1, F2, F3, F4, F5, F6, F7, F8, F9, F10, F11, F12, |
| f-18 | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| floxuridine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluciclovine | F1, F2, F3, F4, F5, F6, F7, F8, F9, F10, F11, F12, |
| f-18 | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluconazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flucytosine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fludarabine | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fludeoxy- | F1, F2, F3, F4, F5, F6, F7, F8, F9, F10, F11, F12, |
| glucose | F13, F14, F15, F16, F17, F18, F19, F20, F21, F22, F23, |
| f-18 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fludro- | F1, F2, F3, F4, F5, F6, F7, F8, F9, F10, F11, F12, |
| cortisone | F13, F14, F15, F16, F17, F18, F19, F20, F21, F22, F23, |
| acetate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flumazenil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flunisolide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluocinolone | F1, F2, F3, F4, F5, F6, F7, F8, F9, F10, F11, F12, |
| acetonide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluocinonide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluorescein | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluoro- | F1, F2, F3, F4, F5, F6, F7, F8, F9, F10, F11, F12, |
| metholone | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluoro- | F1, F2, F3, F4, F5, F6, F7, F8, F9, F10, F11, F12, |
| metholone | F13, F14, F15, F16, F17, F18, F19, F20, F21, F22, F23, |
| acetate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluorouracil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluoxetine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluoxy- | F1, F2, F3, F4, F5, F6, F7, F8, F9, F10, F11, F12, |
| mesterone | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluphenazine | F1, F2, F3, F4, F5, F6, F7, F8, F9, F10, F11, F12, |
| decanoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluphenazine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flur- | F1, F2, F3, F4, F5, F6, F7, F8, F9, F10, F11, F12, |
| andrenolide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flurazepam | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flurbiprofen | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flurbiprofen | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flutamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| flutemetamol | F1, F2, F3, F4, F5, F6, F7, F8, F9, F10, F11, F12, |
| f-18 | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluticasone | F1, F2, F3, F4, F5, F6, F7, F8, F9, F10, F11, F12, |
| furoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluticasone | F1, F2, F3, F4, F5, F6, F7, F8, F9, F10, F11, F12, |
| propionate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluvastatin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fluvoxamine | F1, F2, F3, F4, F5, F6, F7, F8, F9, F10, F11, F12, |
| maleate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| folic acid | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| follitropin | F1, F2, F3, F4, F5, F6, F7, F8, F9, F10, F11, F12, |
| alfa/beta | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fomepizole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fondaparinux | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| formoterol | F1, F2, F3, F4, F5, F6, F7, F8, F9, F10, F11, F12, |
| fumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fosamprenavir | F1, F2, F3, F4, F5, F6, F7, F8, F9, F10, F11, F12, |
| calcium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fosaprepitant | F1, F2, F3, F4, F5, F6, F7, F8, F9, F10, F11, F12, |
| dimeglumine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| foscarnet | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fosfomycin | F1, F2, F3, F4, F5, F6, F7, F8, F9, F10, F11, F12, |
| tromethamine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fosinopril | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fosnetupitant | F1, F2, F3, F4, F5, F6, F7, F8, F9, F10, F11, F12, |
| chloride | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fosphenytoin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fostamatinib | F1, F2, F3, F4, F5, F6, F7, F8, F9, F10, F11, F12, |
| disodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| frovatriptan | F1, F2, F3, F4, F5, F6, F7, F8, F9, F10, F11, F12, |
| succinate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| fulvestrant | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| furosemide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gabapentin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gabapentin | F1, F2, F3, F4, F5, F6, F7, F8, F9, F10, F11, F12, |
| enacarbil | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gadobenate | F1, F2, F3, F4, F5, F6, F7, F8, F9, F10, F11, F12, |
| dimeglumine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gadobutrol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gadodiamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gadopentetate | F1, F2, F3, F4, F5, F6, F7, F8, F9, F10, F11, F12, |
| dimeglumine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gadoterate | F1, F2, F3, F4, F5, F6, F7, F8, F9, F10, F11, F12, |
| meglumine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gadoteridol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gadover- | F1, F2, F3, F4, F5, F6, F7, F8, F9, F10, F11, F12, |
| setamide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gadoxetate | F1, F2, F3, F4, F5, F6, F7, F8, F9, F10, F11, F12, |
| disodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| galantamine | F1, F2, F3, F4, F5, F6, F7, F8, F9, F10, F11, F12, |
| hydrobromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gallium | F1, F2, F3, F4, F5, F6, F7, F8, F9, F10, F11, F12, |
| citrate | F13, F14, F15, F16, F17, F18, F19, F20, F21, F22, F23, |
| ga-67 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gallium | F1, F2, F3, F4, F5, F6, F7, F8, F9, F10, F11, F12, |
| dotatate | F13, F14, F15, F16, F17, F18, F19, F20, F21, F22, F23, |
| ga-68 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ganciclovir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ganciclovir | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ganirelix | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gatifloxacin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gefitinib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gemcitabine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gemfibrozil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gemifloxacin | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gentamicin | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| glacial | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| glatiramer | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| glecaprevir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| glimepiride | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| glipizide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| glucagon | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| glucagon | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| glyburide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| glycerol | F1, F2, F3, F4, F5, F6, F7, F8, F9, F10, F11, F12, |
| phenyl- | F13, F14, F15, F16, F17, F18, F19, F20, F21, F22, F23, |
| butyrate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| glycine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gly- | F1, F2, F3, F4, F5, F6, F7, F8, F9, F10, F11, F12, |
| copyrrolate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| glyco- | F1, F2, F3, F4, F5, F6, F7, F8, F9, F10, F11, F12, |
| pyrronium | F13, F14, F15, F16, F17, F18, F19, F20, F21, F22, F23, |
| tosylate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gonadotropin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| goserelin | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| gramicidin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| granisetron | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| granisetron | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| griseofulvin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| guaifenesin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| guaifenesin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| guanabenz | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| guanfacine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| guanidine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| halcinonide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| halobetasol | F1, F2, F3, F4, F5, F6, F7, F8, F9, F10, F11, F12, |
| propionate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| haloperidol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| haloperidol | F1, F2, F3, F4, F5, F6, F7, F8, F9, F10, F11, F12, |
| decanoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| haloperidol | F1, F2, F3, F4, F5, F6, F7, F8, F9, F10, F11, F12, |
| lactate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| heparin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hexa- | F1, F2, F3, F4, F5, F6, F7, F8, F9, F10, F11, F12, |
| chlorophene | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hexaminole- | F1, F2, F3, F4, F5, F6, F7, F8, F9, F10, F11, F12, |
| vulinate | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| histrelin | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| homatropine | F1, F2, F3, F4, F5, F6, F7, F8, F9, F10, F11, F12, |
| methyl- | F13, F14, F15, F16, F17, F18, F19, F20, F21, F22, F23, |
| bromide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hyaluronidase | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hyaluronidase | F1, F2, F3, F4, F5, F6, F7, F8, F9, F10, F11, F12, |
| recombinant | F13, F14, F15, F16, F17, F18, F19, F20, F21, F22, F23, |
| human | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydralazine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydro- | F1, F2, F3, F4, F5, F6, F7, F8, F9, F10, F11, F12, |
| chloro- | F13, F14, F15, F16, F17, F18, F19, F20, F21, F22, F23, |
| thiazide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydrocodone | F1, F2, F3, F4, F5, F6, F7, F8, F9, F10, F11, F12, |
| bitartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydro- | F1, F2, F3, F4, F5, F6, F7, F8, F9, F10, F11, F12, |
| cortisone | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydro- | F1, F2, F3, F4, F5, F6, F7, F8, F9, F10, F11, F12, |
| cortisone | F13, F14, F15, F16, F17, F18, F19, F20, F21, F22, F23, |
| acetate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydro- | F1, F2, F3, F4, F5, F6, F7, F8, F9, F10, F11, F12, |
| cortisone | F13, F14, F15, F16, F17, F18, F19, F20, F21, F22, F23, |
| butyrate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydro- | F1, F2, F3, F4, F5, F6, F7, F8, F9, F10, F11, F12, |
| cortisone | F13, F14, F15, F16, F17, F18, F19, F20, F21, F22, F23, |
| probutate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydro- | F1, F2, F3, F4, F5, F6, F7, F8, F9, F10, F11, F12, |
| cortisone | F13, F14, F15, F16, F17, F18, F19, F20, F21, F22, F23, |
| sodium | F24, F25, F26, F27, F28, F29, F30, F31, F32, F33, |
| succinate | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydro- | F1, F2, F3, F4, F5, F6, F7, F8, F9, F10, F11, F12, |
| cortisone | F13, F14, F15, F16, F17, F18, F19, F20, F21, F22, F23, |
| valerate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydrogen | F1, F2, F3, F4, F5, F6, F7, F8, F9, F10, F11, F12, |
| peroxide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydro- | F1, F2, F3, F4, F5, F6, F7, F8, F9, F10, F11, F12, |
| morphone | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydroxo- | F1, F2, F3, F4, F5, F6, F7, F8, F9, F10, F11, F12, |
| cobalamin | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydroxy- | F1, F2, F3, F4, F5, F6, F7, F8, F9, F10, F11, F12, |
| amphetamine | F13, F14, F15, F16, F17, F18, F19, F20, F21, F22, F23, |
| hydrobromide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydroxy- | F1, F2, F3, F4, F5, F6, F7, F8, F9, F10, F11, F12, |
| chloroquine | F13, F14, F15, F16, F17, F18, F19, F20, F21, F22, F23, |
| sulfate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydroxy- | F1, F2, F3, F4, F5, F6, F7, F8, F9, F10, F11, F12, |
| progesterone | F13, F14, F15, F16, F17, F18, F19, F20, F21, F22, F23, |
| caproate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydroxy- | F1, F2, F3, F4, F5, F6, F7, F8, F9, F10, F11, F12, |
| propyl | F13, F14, F15, F16, F17, F18, F19, F20, F21, F22, F23, |
| cellulose | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydroxyurea | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydroxyzine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydroxyzine | F1, F2, F3, F4, F5, F6, F7, F8, F9, F10, F11, F12, |
| pamoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ibandronate | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ibrutinib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ibuprofen | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ibuprofen | F1, F2, F3, F4, F5, F6, F7, F8, F9, F10, F11, F12, |
| lysine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ibuprofen | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ibutilide | F1, F2, F3, F4, F5, F6, F7, F8, F9, F10, F11, F12, |
| fumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| icatibant | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| icodextrin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| icosapent | F1, F2, F3, F4, F5, F6, F7, F8, F9, F10, F11, F12, |
| ethyl | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| idarubicin | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| idelalisib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ifosfamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iloperidone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iloprost | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| imatinib | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| imiglucerase | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| imipramine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| imipramine | F1, F2, F3, F4, F5, F6, F7, F8, F9, F10, F11, F12, |
| pamoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| imiquimod | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| inamrinone | F1, F2, F3, F4, F5, F6, F7, F8, F9, F10, F11, F12, |
| lactate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| indacaterol | F1, F2, F3, F4, F5, F6, F7, F8, F9, F10, F11, F12, |
| maleate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| indapamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| indinavir | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| indium | F1, F2, F3, F4, F5, F6, F7, F8, F9, F10, F11, F12, |
| in-111 | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| indium | F1, F2, F3, F4, F5, F6, F7, F8, F9, F10, F11, F12, |
| in-111 | F13, F14, F15, F16, F17, F18, F19, F20, F21, F22, F23, |
| oxyquinoline | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| indium | F1, F2, F3, F4, F5, F6, F7, F8, F9, F10, F11, F12, |
| in-111 | F13, F14, F15, F16, F17, F18, F19, F20, F21, F22, F23, |
| pentetate | F24, F25, F26, F27, F28, F29, F30, F31, F32, F33, |
| disodium | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| indium | F1, F2, F3, F4, F5, F6, F7, F8, F9, F10, F11, F12, |
| in-111 | F13, F14, F15, F16, F17, F18, F19, F20, F21, F22, F23, |
| pentetreotide | F24, F25, F26, F27, F28, F29, F30, F31, F32, F33, |
| kit | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| indocyanine | F1, F2, F3, F4, F5, F6, F7, F8, F9, F10, F11, F12, |
| green | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| indomethacin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| indomethacin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ingenol | F1, F2, F3, F4, F5, F6, F7, F8, F9, F10, F11, F12, |
| mebutate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| aspart | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| aspart | F13, F14, F15, F16, F17, F18, F19, F20, F21, F22, F23, |
| protamine | F24, F25, F26, F27, F28, F29, F30, F31, F32, F33, |
| recombinant | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| aspart | F13, F14, F15, F16, F17, F18, F19, F20, F21, F22, F23, |
| recombinant | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| degludec | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| detemir | F13, F14, F15, F16, F17, F18, F19, F20, F21, F22, F23, |
| recombinant | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| glargine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| glargine | F13, F14, F15, F16, F17, F18, F19, F20, F21, F22, F23, |
| recombinant | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| glulisine | F13, F14, F15, F16, F17, F18, F19, F20, F21, F22, F23, |
| recombinant | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| human | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| lispro | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| lispro | F13, F14, F15, F16, F17, F18, F19, F20, F21, F22, F23, |
| recombinant | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| recombinant | F13, F14, F15, F16, F17, F18, F19, F20, F21, F22, F23, |
| human | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| insulin | F1, F2, F3, F4, F5, F6, F7, F8, F9, F10, F11, F12, |
| susp | F13, F14, F15, F16, F17, F18, F19, F20, F21, F22, F23, |
| isophane | F24, F25, F26, F27, F28, F29, F30, F31, F32, F33, |
| recombinant | F34, F35, F36, F37, F38, F39, F40, F41, F42, F43, |
| human | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iobenguane | F1, F2, F3, F4, F5, F6, F7, F8, F9, F10, F11, F12, |
| sulfate | F13, F14, F15, F16, F17, F18, F19, F20, F21, F22, F23, |
| i-123 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iodine | F1, F2, F3, F4, F5, F6, F7, F8, F9, F10, F11, F12, |
| povacrylex | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iodixanol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ioflupane | F1, F2, F3, F4, F5, F6, F7, F8, F9, F10, F11, F12, |
| i-123 | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iohexol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iopamidol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iopromide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iothalamate | F1, F2, F3, F4, F5, F6, F7, F8, F9, F10, F11, F12, |
| meglumine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iothalamate | F1, F2, F3, F4, F5, F6, F7, F8, F9, F10, F11, F12, |
| sodium | F13, F14, F15, F16, F17, F18, F19, F20, F21, F22, F23, |
| i-125 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ioversol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ipratropium | F1, F2, F3, F4, F5, F6, F7, F8, F9, F10, F11, F12, |
| bromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| irbesartan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| irinotecan | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iron dextran | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| iron sucrose | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| isavuco- | F1, F2, F3, F4, F5, F6, F7, F8, F9, F10, F11, F12, |
| nazonium | F13, F14, F15, F16, F17, F18, F19, F20, F21, F22, F23, |
| sulfate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| isocarboxazid | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| isoflurane | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| isoniazid | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| isoproterenol | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| isosorbide | F1, F2, F3, F4, F5, F6, F7, F8, F9, F10, F11, F12, |
| dinitrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| isosorbide | F1, F2, F3, F4, F5, F6, F7, F8, F9, F10, F11, F12, |
| mononitrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| isosulfan blue | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| isotretinoin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| isradipine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| itraconazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ivabradine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ivacaftor | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ivermectin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ixabepilone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ixazomib | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ketamine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ketoconazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ketoconazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ketoprofen | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ketorolac | F1, F2, F3, F4, F5, F6, F7, F8, F9, F10, F11, F12, |
| trome- | F13, F14, F15, F16, F17, F18, F19, F20, F21, F22, F23, |
| thamine | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ketotifen | F1, F2, F3, F4, F5, F6, F7, F8, F9, F10, F11, F12, |
| fumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| labetalol | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lacosamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lactulose | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lamivudine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lamivudine | F1, F2, F3, F4, F5, F6, F7, F8, F9, F10, F11, F12, |
| and | F13, F14, F15, F16, F17, F18, F19, F20, F21, F22, F23, |
| zidovudine | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lamotrigine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lanreotide | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lansoprazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lanthanum | F1, F2, F3, F4, F5, F6, F7, F8, F9, F10, F11, F12, |
| carbonate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lapatinib | F1, F2, F3, F4, F5, F6, F7, F8, F9, F10, F11, F12, |
| ditosylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| latanoprost | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| latano- | F1, F2, F3, F4, F5, F6, F7, F8, F9, F10, F11, F12, |
| prostene | F13, F14, F15, F16, F17, F18, F19, F20, F21, F22, F23, |
| bunod | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ledipasvir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| leflunomide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lenalidomide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lenvatinib | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lesinurad | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| letermovir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| letrozole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| leucovorin | F1, F2, F3, F4, F5, F6, F7, F8, F9, F10, F11, F12, |
| calcium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| leuprolide | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levalbuterol | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levalbuterol | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levetiracetam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levobunolol | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levocarnitine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levocetirizine | F1, F2, F3, F4, F5, F6, F7, F8, F9, F10, F11, F12, |
| dihydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levocetirizine | F1, F2, F3, F4, F5, F6, F7, F8, F9, F10, F11, F12, |
| dihydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levodopa | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| entacapone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levofloxacin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levoleu- | F1, F2, F3, F4, F5, F6, F7, F8, F9, F10, F11, F12, |
| covorin | F13, F14, F15, F16, F17, F18, F19, F20, F21, F22, F23, |
| calcium | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levomil- | F1, F2, F3, F4, F5, F6, F7, F8, F9, F10, F11, F12, |
| nacipran | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levonordefrin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levonor- | F1, F2, F3, F4, F5, F6, F7, F8, F9, F10, F11, F12, |
| gestrel | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levorphanol | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| levothyroxine | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| l-glutamine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lidocaine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lidocaine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lifitegrast | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| linaclotide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| linagliptin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lincomycin | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lindane | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| linezolid | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| liothyronine | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| liotrix (t4 | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| liraglutide | F1, F2, F3, F4, F5, F6, F7, F8, F9, F10, F11, F12, |
| recombinant | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lisdexam- | F1, F2, F3, F4, F5, F6, F7, F8, F9, F10, F11, F12, |
| fetamine | F13, F14, F15, F16, F17, F18, F19, F20, F21, F22, F23, |
| dimesylate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lisinopril | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lithium | F1, F2, F3, F4, F5, F6, F7, F8, F9, F10, F11, F12, |
| carbonate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lithium | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lixisenatide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lodoxamide | F1, F2, F3, F4, F5, F6, F7, F8, F9, F10, F11, F12, |
| trome- | F13, F14, F15, F16, F17, F18, F19, F20, F21, F22, F23, |
| thamine | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lofexidine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lomitapide | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lomustine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| loperamide | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lopinavir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| loratadine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lorazepam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lorcaserin | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| losartan | F1, F2, F3, F4, F5, F6, F7, F8, F9, F10, F11, F12, |
| potassium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| loteprednol | F1, F2, F3, F4, F5, F6, F7, F8, F9, F10, F11, F12, |
| etabonate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lovastatin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| loxapine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| loxapine | F1, F2, F3, F4, F5, F6, F7, F8, F9, F10, F11, F12, |
| succinate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lubiprostone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| luliconazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lurasidone | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| lutetium | F1, F2, F3, F4, F5, F6, F7, F8, F9, F10, F11, F12, |
| dotatate | F13, F14, F15, F16, F17, F18, F19, F20, F21, F22, F23, |
| lu-177 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| macimorelin | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| macitentan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mafenide | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| magnesium | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| magnesium | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| malathion | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| manganese | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mannitol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| maprotiline | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| maraviroc | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mebendazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mecamyl- | F1, F2, F3, F4, F5, F6, F7, F8, F9, F10, F11, F12, |
| amine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mecasermin | F1, F2, F3, F4, F5, F6, F7, F8, F9, F10, F11, F12, |
| recombinant | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mechlore- | F1, F2, F3, F4, F5, F6, F7, F8, F9, F10, F11, F12, |
| thamine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| meclizine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| meclo- | F1, F2, F3, F4, F5, F6, F7, F8, F9, F10, F11, F12, |
| fenamate | F13, F14, F15, F16, F17, F18, F19, F20, F21, F22, F23, |
| sodium | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| medroxy- | F1, F2, F3, F4, F5, F6, F7, F8, F9, F10, F11, F12, |
| progesterone | F13, F14, F15, F16, F17, F18, F19, F20, F21, F22, F23, |
| acetate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mefenamic | F1, F2, F3, F4, F5, F6, F7, F8, F9, F10, F11, F12, |
| acid | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mefloquine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| megestrol | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| meloxicam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| melphalan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| melphalan | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| memantine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| menotropins | F1, F2, F3, F4, F5, F6, F7, F8, F9, F10, F11, F12, |
| (fsh | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| menthol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| meperidine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mepivacaine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| meprobamate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mercap- | F1, F2, F3, F4, F5, F6, F7, F8, F9, F10, F11, F12, |
| topurine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| meropenem | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mesalamine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mesna | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mestranol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metapro- | F1, F2, F3, F4, F5, F6, F7, F8, F9, F10, F11, F12, |
| terenol | F13, F14, F15, F16, F17, F18, F19, F20, F21, F22, F23, |
| sulfate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metaxalone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metformin | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methacholine | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methadone | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metham- | F1, F2, F3, F4, F5, F6, F7, F8, F9, F10, F11, F12, |
| phetamine | F13, F14, F15, F16, F17, F18, F19, F20, F21, F22, F23, |
| hydro- | F24, F25, F26, F27, F28, F29, F30, F31, F32, F33, |
| chloride | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metha- | F1, F2, F3, F4, F5, F6, F7, F8, F9, F10, F11, F12, |
| zolamide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methenamine | F1, F2, F3, F4, F5, F6, F7, F8, F9, F10, F11, F12, |
| hippurate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methimazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metho- | F1, F2, F3, F4, F5, F6, F7, F8, F9, F10, F11, F12, |
| carbamol | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methohexital | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methotrexate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methotrexate | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methoxsalen | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methsco- | F1, F2, F3, F4, F5, F6, F7, F8, F9, F10, F11, F12, |
| polamine | F13, F14, F15, F16, F17, F18, F19, F20, F21, F22, F23, |
| bromide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| meth- | F1, F2, F3, F4, F5, F6, F7, F8, F9, F10, F11, F12, |
| suximide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methyclo- | F1, F2, F3, F4, F5, F6, F7, F8, F9, F10, F11, F12, |
| thiazide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methyldopa | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methyl- | F1, F2, F3, F4, F5, F6, F7, F8, F9, F10, F11, F12, |
| dopate- | F13, F14, F15, F16, F17, F18, F19, F20, F21, F22, F23, |
| hydro | F24, F25, F26, F27, F28, F29, F30, F31, F32, F33, |
| chloride | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methylene | F1, F2, F3, F4, F5, F6, F7, F8, F9, F10, F11, F12, |
| blue | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methyl- | F1, F2, F3, F4, F5, F6, F7, F8, F9, F10, F11, F12, |
| ergonovine | F13, F14, F15, F16, F17, F18, F19, F20, F21, F22, F23, |
| maleate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methyl- | F1, F2, F3, F4, F5, F6, F7, F8, F9, F10, F11, F12, |
| naltrexone | F13, F14, F15, F16, F17, F18, F19, F20, F21, F22, F23, |
| bromide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methyl | F1, F2, F3, F4, F5, F6, F7, F8, F9, F10, F11, F12, |
| phenidate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methyl- | F1, F2, F3, F4, F5, F6, F7, F8, F9, F10, F11, F12, |
| phenidate | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methyl- | F1, F2, F3, F4, F5, F6, F7, F8, F9, F10, F11, F12, |
| prednisolone | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methyl- | F1, F2, F3, F4, F5, F6, F7, F8, F9, F10, F11, F12, |
| prednisolone | F13, F14, F15, F16, F17, F18, F19, F20, F21, F22, F23, |
| acetate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methyl- | F1, F2, F3, F4, F5, F6, F7, F8, F9, F10, F11, F12, |
| prednisolone | F13, F14, F15, F16, F17, F18, F19, F20, F21, F22, F23, |
| sodium | F24, F25, F26, F27, F28, F29, F30, F31, F32, F33, |
| succinate | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| methyl- | F1, F2, F3, F4, F5, F6, F7, F8, F9, F10, F11, F12, |
| testosterone | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metipranolol | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metoclo- | F1, F2, F3, F4, F5, F6, F7, F8, F9, F10, F11, F12, |
| pramide | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metolazone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metoprolol | F1, F2, F3, F4, F5, F6, F7, F8, F9, F10, F11, F12, |
| succinate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metoprolol | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metronidazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metyrapone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| metyrosine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mexiletine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| micafungin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| miconazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| miconazole | F1, F2, F3, F4, F5, F6, F7, F8, F9, F10, F11, F12, |
| nitrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| midazolam | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| midodrine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| midostaurin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mifepristone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| miglitol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| miglustat | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| milnacipran | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| milrinone | F1, F2, F3, F4, F5, F6, F7, F8, F9, F10, F11, F12, |
| lactate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| miltefosine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| minocycline | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| minoxidil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mirabegron | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mirtazapine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| misoprostol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mitomycin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mitotane | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mitoxantrone | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| modafinil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| moexipril | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| molindone | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mometasone | F1, F2, F3, F4, F5, F6, F7, F8, F9, F10, F11, F12, |
| furoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| montelukast | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| morphine | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| moxidectin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| moxifloxacin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| moxifloxacin | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mupirocin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| mupirocin | F1, F2, F3, F4, F5, F6, F7, F8, F9, F10, F11, F12, |
| calcium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| myco- | F1, F2, F3, F4, F5, F6, F7, F8, F9, F10, F11, F12, |
| phenolate | F13, F14, F15, F16, F17, F18, F19, F20, F21, F22, F23, |
| mofetil | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| myco- | F1, F2, F3, F4, F5, F6, F7, F8, F9, F10, F11, F12, |
| phenolate | F13, F14, F15, F16, F17, F18, F19, F20, F21, F22, F23, |
| mofetil | F24, F25, F26, F27, F28, F29, F30, F31, F32, F33, |
| hydro- | F34, F35, F36, F37, F38, F39, F40, F41, F42, F43, |
| chloride | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| myco- | F1, F2, F3, F4, F5, F6, F7, F8, F9, F10, F11, F12, |
| phenolic | F13, F14, F15, F16, F17, F18, F19, F20, F21, F22, F23, |
| acid | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nabilone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nabumetone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nadolol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nafarelin | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nafcillin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| naftifine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nalbuphine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| naldemedine | F1, F2, F3, F4, F5, F6, F7, F8, F9, F10, F11, F12, |
| tosylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| naloxegol | F1, F2, F3, F4, F5, F6, F7, F8, F9, F10, F11, F12, |
| oxalate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| naloxone | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| naltrexone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| naltrexone | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nandrolone | F1, F2, F3, F4, F5, F6, F7, F8, F9, F10, F11, F12, |
| decanoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| naphazoline | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| naproxen | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| naproxen | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| naratriptan | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| natamycin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nateglinide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nebivolol | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nedocromil | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nefazodone | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nelarabine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nelfinavir | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| neomycin | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| neostigmine | F1, F2, F3, F4, F5, F6, F7, F8, F9, F10, F11, F12, |
| methyl- | F13, F14, F15, F16, F17, F18, F19, F20, F21, F22, F23, |
| sulfate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nepafenac | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| neratinib | F1, F2, F3, F4, F5, F6, F7, F8, F9, F10, F11, F12, |
| maleate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nesiritide | F1, F2, F3, F4, F5, F6, F7, F8, F9, F10, F11, F12, |
| recombinant | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| netarsudil | F1, F2, F3, F4, F5, F6, F7, F8, F9, F10, F11, F12, |
| dimesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| netupitant | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nevirapine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| niacin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nicardipine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nicotine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nicotine | F1, F2, F3, F4, F5, F6, F7, F8, F9, F10, F11, F12, |
| polacrilex | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nifedipine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nilotinib | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nilutamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nimodipine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nintedanib | F1, F2, F3, F4, F5, F6, F7, F8, F9, F10, F11, F12, |
| esylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| niraparib | F1, F2, F3, F4, F5, F6, F7, F8, F9, F10, F11, F12, |
| tosylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nisoldipine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nitazoxanide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nitisinone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nitrofurantoin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nitroglycerin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nizatidine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nonoxynol-9 | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nor- | F1, F2, F3, F4, F5, F6, F7, F8, F9, F10, F11, F12, |
| epinephrine | F13, F14, F15, F16, F17, F18, F19, F20, F21, F22, F23, |
| bitartrate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| norethindrone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| norethin- | F1, F2, F3, F4, F5, F6, F7, F8, F9, F10, F11, F12, |
| drone | F13, F14, F15, F16, F17, F18, F19, F20, F21, F22, F23, |
| acetate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nortriptyline | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nusinersen | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| nystatin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| obeticholic | F1, F2, F3, F4, F5, F6, F7, F8, F9, F10, F11, F12, |
| acid | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| octreotide | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ofloxacin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| olanzapine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| olanzapine | F1, F2, F3, F4, F5, F6, F7, F8, F9, F10, F11, F12, |
| pamoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| olaparib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| olmesartan | F1, F2, F3, F4, F5, F6, F7, F8, F9, F10, F11, F12, |
| medoxomil | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| olodaterol | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| olopatadine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| olsalazine | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| omacetaxine | F1, F2, F3, F4, F5, F6, F7, F8, F9, F10, F11, F12, |
| mepe- | F13, F14, F15, F16, F17, F18, F19, F20, F21, F22, F23, |
| succinate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ombitasvir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| omega- | F1, F2, F3, F4, F5, F6, F7, F8, F9, F10, F11, F12, |
| 3-acid | F13, F14, F15, F16, F17, F18, F19, F20, F21, F22, F23, |
| ethyl esters | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| omeprazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| omeprazole | F1, F2, F3, F4, F5, F6, F7, F8, F9, F10, F11, F12, |
| magnesium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ondansetron | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ondansetron | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oritavancin | F1, F2, F3, F4, F5, F6, F7, F8, F9, F10, F11, F12, |
| diphosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| orlistat | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| orphenadrine | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oseltamivir | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| osimertinib | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ospemifene | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxacillin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxaliplatin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxandrolone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxaprozin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxazepam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxcar- | F1, F2, F3, F4, F5, F6, F7, F8, F9, F10, F11, F12, |
| bazepine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxiconazole | F1, F2, F3, F4, F5, F6, F7, F8, F9, F10, F11, F12, |
| nitrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxtriphylline | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxybutynin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxybutynin | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxycodone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxycodone | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxy- | F1, F2, F3, F4, F5, F6, F7, F8, F9, F10, F11, F12, |
| metazoline | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxymetholone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxymor- | F1, F2, F3, F4, F5, F6, F7, F8, F9, F10, F11, F12, |
| phone | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxytetra- | F1, F2, F3, F4, F5, F6, F7, F8, F9, F10, F11, F12, |
| cycline | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| oxytocin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ozenoxacin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| paclitaxel | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| palbociclib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| paliperidone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| paliperidone | F1, F2, F3, F4, F5, F6, F7, F8, F9, F10, F11, F12, |
| palmitate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| palonosetron | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pamidronate | F1, F2, F3, F4, F5, F6, F7, F8, F9, F10, F11, F12, |
| disodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pancrelipase | F1, F2, F3, F4, F5, F6, F7, F8, F9, F10, F11, F12, |
| (amylase | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pancuronium | F1, F2, F3, F4, F5, F6, F7, F8, F9, F10, F11, F12, |
| bromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| panobinostat | F1, F2, F3, F4, F5, F6, F7, F8, F9, F10, F11, F12, |
| lactate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pantoprazole | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| paricalcitol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| paromo- | F1, F2, F3, F4, F5, F6, F7, F8, F9, F10, F11, F12, |
| mycin | F13, F14, F15, F16, F17, F18, F19, F20, F21, F22, F23, |
| sulfate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| paroxetine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| paroxetine | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pasireotide | F1, F2, F3, F4, F5, F6, F7, F8, F9, F10, F11, F12, |
| diaspartate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pasireotide | F1, F2, F3, F4, F5, F6, F7, F8, F9, F10, F11, F12, |
| pamoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| patiromer | F1, F2, F3, F4, F5, F6, F7, F8, F9, F10, F11, F12, |
| sorbitex | F13, F14, F15, F16, F17, F18, F19, F20, F21, F22, F23, |
| calcium | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pazopanib | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pegademase | F1, F2, F3, F4, F5, F6, F7, F8, F9, F10, F11, F12, |
| bovine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pegaptanib | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pegvisomant | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pemetrexed | F1, F2, F3, F4, F5, F6, F7, F8, F9, F10, F11, F12, |
| disodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| penciclovir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| peni- | F1, F2, F3, F4, F5, F6, F7, F8, F9, F10, F11, F12, |
| cillamine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| penicillin g | F1, F2, F3, F4, F5, F6, F7, F8, F9, F10, F11, F12, |
| benzathine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| penicillin g | F1, F2, F3, F4, F5, F6, F7, F8, F9, F10, F11, F12, |
| potassium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| penicillin g | F1, F2, F3, F4, F5, F6, F7, F8, F9, F10, F11, F12, |
| procaine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| penicillin g | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| penicillin v | F1, F2, F3, F4, F5, F6, F7, F8, F9, F10, F11, F12, |
| potassium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pentamidine | F1, F2, F3, F4, F5, F6, F7, F8, F9, F10, F11, F12, |
| isethionate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pentetate | F1, F2, F3, F4, F5, F6, F7, F8, F9, F10, F11, F12, |
| calcium | F13, F14, F15, F16, F17, F18, F19, F20, F21, F22, F23, |
| trisodium | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pentetate | F1, F2, F3, F4, F5, F6, F7, F8, F9, F10, F11, F12, |
| zinc | F13, F14, F15, F16, F17, F18, F19, F20, F21, F22, F23, |
| trisodium | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pento- | F1, F2, F3, F4, F5, F6, F7, F8, F9, F10, F11, F12, |
| barbital | F13, F14, F15, F16, F17, F18, F19, F20, F21, F22, F23, |
| sodium | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pentosan | F1, F2, F3, F4, F5, F6, F7, F8, F9, F10, F11, F12, |
| polysulfate | F13, F14, F15, F16, F17, F18, F19, F20, F21, F22, F23, |
| sodium | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pentostatin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pentoxi- | F1, F2, F3, F4, F5, F6, F7, F8, F9, F10, F11, F12, |
| fylline | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| peramivir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| perampanel | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| perflutren | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| perindopril | F1, F2, F3, F4, F5, F6, F7, F8, F9, F10, F11, F12, |
| erbumine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| permethrin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| perphenazine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| phendi- | F1, F2, F3, F4, F5, F6, F7, F8, F9, F10, F11, F12, |
| metrazine | F13, F14, F15, F16, F17, F18, F19, F20, F21, F22, F23, |
| tartrate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| phenelzine | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| phenoxy- | F1, F2, F3, F4, F5, F6, F7, F8, F9, F10, F11, F12, |
| benzamine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| phentermine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| phentol- | F1, F2, F3, F4, F5, F6, F7, F8, F9, F10, F11, F12, |
| amine | F13, F14, F15, F16, F17, F18, F19, F20, F21, F22, F23, |
| mesylate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| phenyl- | F1, F2, F3, F4, F5, F6, F7, F8, F9, F10, F11, F12, |
| ephrine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| codeine | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| phenytoin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| phenytoin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| phytonadione | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pilocarpine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pimavanserin | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pimecrolimus | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pimozide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pindolol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pioglitazone | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| piperacillin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pirfenidone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| piroxicam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pitavastatin | F1, F2, F3, F4, F5, F6, F7, F8, F9, F10, F11, F12, |
| calcium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pitavastatin | F1, F2, F3, F4, F5, F6, F7, F8, F9, F10, F11, F12, |
| magnesium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| plazomicin | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| plecanatide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| plerixafor | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| podofilox | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| polidocanol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| polyethylene | F1, F2, F3, F4, F5, F6, F7, F8, F9, F10, F11, F12, |
| glycol 3350 | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| polymyxin b | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pomalidomide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ponatinib | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| poractant | F1, F2, F3, F4, F5, F6, F7, F8, F9, F10, F11, F12, |
| alfa | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| porfimer | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| posaconazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| potassium | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| potassium | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| potassium | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| potassium | F1, F2, F3, F4, F5, F6, F7, F8, F9, F10, F11, F12, |
| iodide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| povidone- | F1, F2, F3, F4, F5, F6, F7, F8, F9, F10, F11, F12, |
| iodine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pralatrexate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pralidoxime | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pramipexole | F1, F2, F3, F4, F5, F6, F7, F8, F9, F10, F11, F12, |
| dihydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pramlintide | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prasterone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prasugrel | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pravastatin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| praziquantel | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prazosin | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prednicarbate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prednisolone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prednisolone | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prednisolone | F1, F2, F3, F4, F5, F6, F7, F8, F9, F10, F11, F12, |
| sodium | F13, F14, F15, F16, F17, F18, F19, F20, F21, F22, F23, |
| phosphate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prednisone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pregabalin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prilocaine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| primaquine | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| primidone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| probenecid | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| procainamide | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| procarbazine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prochlor- | F1, F2, F3, F4, F5, F6, F7, F8, F9, F10, F11, F12, |
| perazine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prochlor- | F1, F2, F3, F4, F5, F6, F7, F8, F9, F10, F11, F12, |
| perazine | F13, F14, F15, F16, F17, F18, F19, F20, F21, F22, F23, |
| edisylate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| prochlor- | F1, F2, F3, F4, F5, F6, F7, F8, F9, F10, F11, F12, |
| perazine | F13, F14, F15, F16, F17, F18, F19, F20, F21, F22, F23, |
| maleate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| progesterone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| promethazine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| propafenone | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pro- | F1, F2, F3, F4, F5, F6, F7, F8, F9, F10, F11, F12, |
| pantheline | F13, F14, F15, F16, F17, F18, F19, F20, F21, F22, F23, |
| bromide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| proparacaine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| propofol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| propranolol | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| propyl- | F1, F2, F3, F4, F5, F6, F7, F8, F9, F10, F11, F12, |
| thiouracil | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| protamine | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| protriptyline | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pseudo- | F1, F2, F3, F4, F5, F6, F7, F8, F9, F10, F11, F12, |
| ephedrine | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| dextro- | F1, F2, F3, F4, F5, F6, F7, F8, F9, F10, F11, F12, |
| methorphan | F13, F14, F15, F16, F17, F18, F19, F20, F21, F22, F23, |
| hydro- | F24, F25, F26, F27, F28, F29, F30, F31, F32, F33, |
| bromide | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pseudo- | F1, F2, F3, F4, F5, F6, F7, F8, F9, F10, F11, F12, |
| ephedrine | F13, F14, F15, F16, F17, F18, F19, F20, F21, F22, F23, |
| sulfate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pyrazinamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pyri- | F1, F2, F3, F4, F5, F6, F7, F8, F9, F10, F11, F12, |
| dostigmine | F13, F14, F15, F16, F17, F18, F19, F20, F21, F22, F23, |
| bromide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pyridoxine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| pyrime- | F1, F2, F3, F4, F5, F6, F7, F8, F9, F10, F11, F12, |
| thamine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| quazepam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| quetiapine | F1, F2, F3, F4, F5, F6, F7, F8, F9, F10, F11, F12, |
| fumarate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| quinapril | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| quinidine | F1, F2, F3, F4, F5, F6, F7, F8, F9, F10, F11, F12, |
| gluconate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| quinidine | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| quinine | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rabeprazole | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| radium | F1, F2, F3, F4, F5, F6, F7, F8, F9, F10, F11, F12, |
| ra-223 | F13, F14, F15, F16, F17, F18, F19, F20, F21, F22, F23, |
| dichloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| raloxifene | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| raltegravir | F1, F2, F3, F4, F5, F6, F7, F8, F9, F10, F11, F12, |
| potassium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ramelteon | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ramipril | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ranitidine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ranolazine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rasagiline | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| regadenoson | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| regorafenib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| remifentanil | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| repaglinide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| retapamulin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ribavirin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ribociclib | F1, F2, F3, F4, F5, F6, F7, F8, F9, F10, F11, F12, |
| succinate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| riboflavin 5′- | F1, F2, F3, F4, F5, F6, F7, F8, F9, F10, F11, F12, |
| phosphate | F13, F14, F15, F16, F17, F18, F19, F20, F21, F22, F23, |
| sodium | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rifabutin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rifampin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rifapentine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rifaximin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rilpivirine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| riluzole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rimantadine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| riociguat | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| risedronate | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| risperidone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ritonavir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rivaroxaban | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rivastigmine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rivastigmine | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rizatriptan | F1, F2, F3, F4, F5, F6, F7, F8, F9, F10, F11, F12, |
| benzoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rocuronium | F1, F2, F3, F4, F5, F6, F7, F8, F9, F10, F11, F12, |
| bromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rofecoxib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| roflumilast | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rolapitant | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| romidepsin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ropinirole | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ropivacaine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rosiglitazone | F1, F2, F3, F4, F5, F6, F7, F8, F9, F10, F11, F12, |
| maleate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rosuvastatin | F1, F2, F3, F4, F5, F6, F7, F8, F9, F10, F11, F12, |
| calcium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rotigotine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rubidium | F1, F2, F3, F4, F5, F6, F7, F8, F9, F10, F11, F12, |
| chloride | F13, F14, F15, F16, F17, F18, F19, F20, F21, F22, F23, |
| rb-82 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rucaparib | F1, F2, F3, F4, F5, F6, F7, F8, F9, F10, F11, F12, |
| camsylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| rufinamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ruxolitinib | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sacrosidase | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sacubitril | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| safinamide | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| salmeterol | F1, F2, F3, F4, F5, F6, F7, F8, F9, F10, F11, F12, |
| xinafoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| samarium | F1, F2, F3, F4, F5, F6, F7, F8, F9, F10, F11, F12, |
| sm-153 | F13, F14, F15, F16, F17, F18, F19, F20, F21, F22, F23, |
| lexidronam | F24, F25, F26, F27, F28, F29, F30, F31, F32, F33, |
| pentasodium | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sapropterin | F1, F2, F3, F4, F5, F6, F7, F8, F9, F10, F11, F12, |
| dihydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| saquinavir | F1, F2, F3, F4, F5, F6, F7, F8, F9, F10, F11, F12, |
| | F13, F14, F15, F16, F17, F18, F19, F20, F21, F22, F23, |
| mesylate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| saxagliptin | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| scopolamine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| secnidazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| secobarbital | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| secretin | F1, F2, F3, F4, F5, F6, F7, F8, F9, F10, F11, F12, |
| synthetic | F13, F14, F15, F16, F17, F18, F19, F20, F21, F22, F23, |
| human | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| selegiline | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| selegiline | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| selenium s | F1, F2, F3, F4, F5, F6, F7, F8, F9, F10, F11, F12, |
| ulfide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| selexipag | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| semaglutide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sertaconazole | F1, F2, F3, F4, F5, F6, F7, F8, F9, F10, F11, F12, |
| nitrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sertraline | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sevelamer | F1, F2, F3, F4, F5, F6, F7, F8, F9, F10, F11, F12, |
| carbonate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sevelamer | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sevoflurane | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sildenafil | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| silodosin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| silver | F1, F2, F3, F4, F5, F6, F7, F8, F9, F10, F11, F12, |
| sulfadiazine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| simvastatin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sincalide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sinecatechins | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sirolimus | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sitagliptin | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| benzoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| bicarbonate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| ferric | F13, F14, F15, F16, F17, F18, F19, F20, F21, F22, F23, |
| gluconate | F24, F25, F26, F27, F28, F29, F30, F31, F32, F33, |
| complex | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| fluoride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| fluoride | F13, F14, F15, F16, F17, F18, F19, F20, F21, F22, F23, |
| f-18 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| iodide | F13, F14, F15, F16, F17, F18, F19, F20, F21, F22, F23, |
| i-123 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| iodide | F13, F14, F15, F16, F17, F18, F19, F20, F21, F22, F23, |
| i-131 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| lactate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| nitrite | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| nitro- | F13, F14, F15, F16, F17, F18, F19, F20, F21, F22, F23, |
| prusside | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| oxybate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| phenyl- | F13, F14, F15, F16, F17, F18, F19, F20, F21, F22, F23, |
| butyrate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| polystyrene | F13, F14, F15, F16, F17, F18, F19, F20, F21, F22, F23, |
| sulfonate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| tetradecyl | F13, F14, F15, F16, F17, F18, F19, F20, F21, F22, F23, |
| sulfate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| thiosulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sodium | F1, F2, F3, F4, F5, F6, F7, F8, F9, F10, F11, F12, |
| zirconium | F13, F14, F15, F16, F17, F18, F19, F20, F21, F22, F23, |
| cyclosilicate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sofosbuvir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| solifenacin | F1, F2, F3, F4, F5, F6, F7, F8, F9, F10, F11, F12, |
| succinate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| somatropin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| somatropin | F1, F2, F3, F4, F5, F6, F7, F8, F9, F10, F11, F12, |
| recombinant | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sonidegib | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sorafenib | F1, F2, F3, F4, F5, F6, F7, F8, F9, F10, F11, F12, |
| tosylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sorbitol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sotalol | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| soybean oil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| spinosad | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| spiro- | F1, F2, F3, F4, F5, F6, F7, F8, F9, F10, F11, F12, |
| nolactone | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| stavudine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| streptomycin | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| streptozocin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| strontium | F1, F2, F3, F4, F5, F6, F7, F8, F9, F10, F11, F12, |
| chloride | F13, F14, F15, F16, F17, F18, F19, F20, F21, F22, F23, |
| sr-89 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| succimer | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| succinyl- | F1, F2, F3, F4, F5, F6, F7, F8, F9, F10, F11, F12, |
| choline | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sucralfate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sucroferric | F1, F2, F3, F4, F5, F6, F7, F8, F9, F10, F11, F12, |
| oxyhy- | F13, F14, F15, F16, F17, F18, F19, F20, F21, F22, F23, |
| droxide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sufentanil | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sugammadex | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sulbactam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sulconazole | F1, F2, F3, F4, F5, F6, F7, F8, F9, F10, F11, F12, |
| nitrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sulfacetamide | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sulfadiazine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sulfame- | F1, F2, F3, F4, F5, F6, F7, F8, F9, F10, F11, F12, |
| thoxazole | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sulfanilamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sulfasalazine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sulfur | F1, F2, F3, F4, F5, F6, F7, F8, F9, F10, F11, F12, |
| hexafluoride | F13, F14, F15, F16, F17, F18, F19, F20, F21, F22, F23, |
| lipid-type a | F24, F25, F26, F27, F28, F29, F30, F31, F32, F33, |
| microspheres | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sulindac | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sumatriptan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sumatriptan | F1, F2, F3, F4, F5, F6, F7, F8, F9, F10, F11, F12, |
| succinate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| sunitinib | F1, F2, F3, F4, F5, F6, F7, F8, F9, F10, F11, F12, |
| malate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| suvorexant | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tacrolimus | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tadalafil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tafluprost | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| talc | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| taliglucerase | F1, F2, F3, F4, F5, F6, F7, F8, F9, F10, F11, F12, |
| alfa | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tamoxifen | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tamsulosin | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tapentadol | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tasimelteon | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tavaborole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tazarotene | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| albumin | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| bicisate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| disofenin | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| exametazime | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| mebrofenin | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| medronate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| mertiatide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| oxidronate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| pentetate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| pyro- | F24, F25, F26, F27, F28, F29, F30, F31, F32, F33, |
| phosphate | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| sestamibi kit | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| sodium | F24, F25, F26, F27, F28, F29, F30, F31, F32, F33, |
| pertechnetate | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| sulfur | F24, F25, F26, F27, F28, F29, F30, F31, F32, F33, |
| colloid | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| tetrofosmin | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| technetium | F1, F2, F3, F4, F5, F6, F7, F8, F9, F10, F11, F12, |
| tc-99m | F13, F14, F15, F16, F17, F18, F19, F20, F21, F22, F23, |
| tilmanocept | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tedizolid | F1, F2, F3, F4, F5, F6, F7, F8, F9, F10, F11, F12, |
| phosphate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| teduglutide | F1, F2, F3, F4, F5, F6, F7, F8, F9, F10, F11, F12, |
| recombinant | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| telavancin | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| telmisartan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| telotristat | F1, F2, F3, F4, F5, F6, F7, F8, F9, F10, F11, F12, |
| etiprate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| temazepam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| temozolomide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| temsirolimus | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tenofovir | F1, F2, F3, F4, F5, F6, F7, F8, F9, F10, F11, F12, |
| alafenamide | F13, F14, F15, F16, F17, F18, F19, F20, F21, F22, F23, |
| fumarate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tenofovir | F1, F2, F3, F4, F5, F6, F7, F8, F9, F10, F11, F12, |
| disoproxil | F13, F14, F15, F16, F17, F18, F19, F20, F21, F22, F23, |
| fumarate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| terazosin | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| terbinafine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| terbinafine | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| terbutaline | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| terconazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| teriflunomide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| teriparatide | F1, F2, F3, F4, F5, F6, F7, F8, F9, F10, F11, F12, |
| recombinant | F13, F14, F15, F16, F17, F18, F19, F20, F21, F22, F23, |
| human | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tesamorelin | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| testosterone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| testosterone | F1, F2, F3, F4, F5, F6, F7, F8, F9, F10, F11, F12, |
| cypionate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| testosterone | F1, F2, F3, F4, F5, F6, F7, F8, F9, F10, F11, F12, |
| enanthate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| testosterone | F1, F2, F3, F4, F5, F6, F7, F8, F9, F10, F11, F12, |
| undecanoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tetrabenazine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tetracaine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tetracycline | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tetra- | F1, F2, F3, F4, F5, F6, F7, F8, F9, F10, F11, F12, |
| hydrozoline | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| thalidomide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| thallous | F1, F2, F3, F4, F5, F6, F7, F8, F9, F10, F11, F12, |
| chloride | F13, F14, F15, F16, F17, F18, F19, F20, F21, F22, F23, |
| tl-201 | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| theophylline | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| thiamine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| thioguanine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| thioridazine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| thiotepa | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| thiothixene | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| thyrotropin | F1, F2, F3, F4, F5, F6, F7, F8, F9, F10, F11, F12, |
| alfa | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tiagabine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ticagrelor | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ticlopidine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tigecycline | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| timolol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| timolol | F1, F2, F3, F4, F5, F6, F7, F8, F9, F10, F11, F12, |
| maleate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tinidazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tioconazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tiopronin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tiotropium | F1, F2, F3, F4, F5, F6, F7, F8, F9, F10, F11, F12, |
| bromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tipiracil | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tipranavir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tirofiban | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tizanidine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tobramycin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tobramycin | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tofacitinib | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tolazamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tolbutamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tolcapone | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tolmetin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tolterodine | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tolvaptan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| topiramate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| topotecan | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| toremifene | F1, F2, F3, F4, F5, F6, F7, F8, F9, F10, F11, F12, |
| citrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| torsemide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trabectedin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tramadol | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trametinib | F1, F2, F3, F4, F5, F6, F7, F8, F9, F10, F11, F12, |
| dimethyl | F13, F14, F15, F16, F17, F18, F19, F20, F21, F22, F23, |
| sulfoxide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trandolapril | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tranexamic | F1, F2, F3, F4, F5, F6, F7, F8, F9, F10, F11, F12, |
| acid | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tranyl- | F1, F2, F3, F4, F5, F6, F7, F8, F9, F10, F11, F12, |
| cypromine | F13, F14, F15, F16, F17, F18, F19, F20, F21, F22, F23, |
| sulfate | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| travoprost | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trazodone | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| treprostinil | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| treprostinil | F1, F2, F3, F4, F5, F6, F7, F8, F9, F10, F11, F12, |
| diolamine | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tretinoin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| triamcinolone | F1, F2, F3, F4, F5, F6, F7, F8, F9, F10, F11, F12, |
| acetonide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| triamcinolone | F1, F2, F3, F4, F5, F6, F7, F8, F9, F10, F11, F12, |
| hexacetonide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| triamterene | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| triazolam | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trientine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trifluoperazine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trifluridine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trihexy- | F1, F2, F3, F4, F5, F6, F7, F8, F9, F10, F11, F12, |
| phenidyl | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trimethadione | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trimetho- | F1, F2, F3, F4, F5, F6, F7, F8, F9, F10, F11, F12, |
| benzamide | F13, F14, F15, F16, F17, F18, F19, F20, F21, F22, F23, |
| hydrochloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trimethoprim | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trimethoprim | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trimipramine | F1, F2, F3, F4, F5, F6, F7, F8, F9, F10, F11, F12, |
| maleate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| triptorelin | F1, F2, F3, F4, F5, F6, F7, F8, F9, F10, F11, F12, |
| pamoate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tromethamine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| tropicamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trospium | F1, F2, F3, F4, F5, F6, F7, F8, F9, F10, F11, F12, |
| chloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| trypan blue | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ulipristal | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ultramicrosize | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| umeclidinium | F1, F2, F3, F4, F5, F6, F7, F8, F9, F10, F11, F12, |
| bromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| urea | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| uridine | F1, F2, F3, F4, F5, F6, F7, F8, F9, F10, F11, F12, |
| triacetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ursodiol | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| valacyclovir | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| valbenazine | F1, F2, F3, F4, F5, F6, F7, F8, F9, F10, F11, F12, |
| tosylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| valganciclovir | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| valproate | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| valproic acid | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| valrubicin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| valsartan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| hydro- | F1, F2, F3, F4, F5, F6, F7, F8, F9, F10, F11, F12, |
| chlorothiazide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vancomycin | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vandetanib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vardenafil | F1, F2, F3, F4, F5, F6, F7, F8, F9, F10, F11, F12, |
| hydro- | F13, F14, F15, F16, F17, F18, F19, F20, F21, F22, F23, |
| chloride | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| varenicline | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vasopressin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vecuronium | F1, F2, F3, F4, F5, F6, F7, F8, F9, F10, F11, F12, |
| bromide | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| velaglucerase | F1, F2, F3, F4, F5, F6, F7, F8, F9, F10, F11, F12, |
| alfa | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vemurafenib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| venetoclax | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| venlafaxine | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| verapamil | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| verteporfin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vigabatrin | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vilazodone | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vinblastine | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vincristine | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vinorelbine | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vismodegib | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vitamin a | F1, F2, F3, F4, F5, F6, F7, F8, F9, F10, F11, F12, |
| palmitate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vorapaxar | F1, F2, F3, F4, F5, F6, F7, F8, F9, F10, F11, F12, |
| sulfate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| voriconazole | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vorinostat | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| vortioxetine | F1, F2, F3, F4, F5, F6, F7, F8, F9, F10, F11, F12, |
| hydro- | F13, F14, F15, F16, F17, F18, F19, F20, F21, F22, F23, |
| bromide | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| warfarin | F1, F2, F3, F4, F5, F6, F7, F8, F9, F10, F11, F12, |
| sodium | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| xenon xe-133 | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| zafirlukast | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| zaleplon | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| zanamivir | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ziconotide | F1, F2, F3, F4, F5, F6, F7, F8, F9, F10, F11, F12, |
| acetate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| zidovudine | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| zileuton | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| zinc acetate | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| zinc chloride | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ziprasidone | F1, F2, F3, F4, F5, F6, F7, F8, F9, F10, F11, F12, |
| hydrochloride | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| ziprasidone | F1, F2, F3, F4, F5, F6, F7, F8, F9, F10, F11, F12, |
| mesylate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| zoledronic | F1, F2, F3, F4, F5, F6, F7, F8, F9, F10, F11, F12, |
| acid | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| zolmitriptan | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| zolpidem | F1, F2, F3, F4, F5, F6, F7, F8, F9, F10, F11, F12, |
| tartrate | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |
| zonisamide | F1, F2, F3, F4, F5, F6, F7, F8, F9, F10, F11, F12, |
| | F13, F14, F15, F16, F17, F18, F19, F20, F21, F22, F23, |
| | F24, F25, F26, F27, F28, F29, F30, F31, F32, F33, |
| | F34, F35, F36, F37, F38, F39, F40, F41, F42, F43, |
| | F44, F45, F46, F47, F48, F49, F50, F51, F52, F53, |
| | F54, F55, F56, F57, F58, F59, F60, F61, F62, F63, |
| | F64, F65, F66, F67, F68, F69, F70, F71, F72, F73, |
| | F74, F75, F76, F77, F78, F79, F80, F81, F82, F83, |
| | F84, F85, F86, F87, F88, F89, F90, F91, F92, F93, |
| | F94, F95, F96, F97, F98, F99, or F100 |

Depending upon the active pharmaceutical ingredient, the dosage form may be used to treat, or provide relief of, any type of disease or condition. In some embodiments, a dosage form of Table 2 is used to treat a condition including, but not limited to, 16p11.2 Duplication; Azoospermia; Churg-Strauss Syndrome; Edema; Neuroaxonal Dystrophies; Hypoxanthine Guanine Phosphoribosyltransferase Deficiency; Schizophrenia, Disorganized; Glaucoma, Open-Angle; Plasma Cell Granuloma, Pulmonary; Lymphoma, Large-Cell, Immunoblastic; Tooth Fractures; Amyotrophic Lateral Sclerosis Type 11; Carcinoma, Basal Cell; De Quervain Disease; Familial Joint Instability Syndrome; Pain Insensitivity, Congenital; Lactose Intolerance; Spondylolisthesis; Hereditary Angioedema Types I and II; Pulmonary Alveolar Proteinosis; Mucolipidoses; Vertebrobasilar Insufficiency; 21-hydroxylase Deficiency; Azotemia; Chylothorax; Edema, Cardiac; Neurobehavioral Manifestations; Hypoxia; Schizophrenia, Paranoid; Glioblastoma; Plasma Cell Leukemia; Lymphoma, Mantle-Cell; Tooth Injuries; Anal Cancer; Carcinoma, Basosquamous; Deafness; Familial Lipoprotein Lipase Deficiency; Pain, Intractable; Lafora Disease; Spondylolysis; Hereditary Antithrombin Deficiency; Pulmonary Arterial Hypertension; Mucolipidosis III Alpha/beta; Vertigo; 22q11 Deletion Syndrome; B Cell Prolymphocytic Leukemia; Chylothorax, Congenital; Egg Hypersensitivity; Neuroblastoma; Hypoxia, Brain; Schizotypal Personality Disorder; Glioma; Plasmablastic Lymphoma; Lymphoma, Non-Hodgkin; Tooth Loss; Anaphylaxis; Carcinoma, Bronchogenic; Death; Familial Mediterranean Fever; Pain, Postoperative; Lambert-Eaton Myasthenic Syndrome; Spondylosis; Hereditary Antithrombin Deficiency Type 2; Pulmonary Arteriovenous Malformation; Mucolipidosis Type 4; Vesico-Ureteral Reflux; 22q11.2 Deletion Syndrome; B-cell Lymphoma; Cicatricial Pemphigoid; Ehlers-Danlos Syndrome; Neurocognitive Disorders; Hypoxia-Ischemia, Brain; Schnitzler Syndrome; Glioma, Subependymal; Plasmacytoma; Lymphoma, Primary Cutaneous Anaplastic Large Cell; Tooth Migration; Anaplasia; Carcinoma, Ductal; Death, Sudden; Familial Multiple Lipomatosis; Pain, Procedural; Lamellar Ichthyosis; Spontaneous Coronary Artery Dissection; Hereditary Ataxia; Pulmonary Aspergillosis; Mucopolysaccharidoses; Vesicovaginal Fistula; 22q13.3 Deletion Syndrome; Babesiosis; Cicatrix; Eisenmenger Complex; Neurocysticercosis; Hysteria; Schwannoma; Gliomatosis Cerebri; Platelet Storage Pool Deficiency; Lymphoma, Primary Effusion; Tooth Mobility; Anaplasmosis; Carcinoma, Ductal, Breast; Death, Sudden, Cardiac; Familial Pancreatic Cancer; Pain, Referred; Landau-Kleffner Syndrome; Sporotrichosis; Hereditary Autoinflammatory Diseases; Pulmonary Atelectasis; Mucopolysaccharidosis I; Vesicular Stomatitis; 3q29 Microdeletion Syndrome; Back Injuries; Cicatrix, Hypertrophic; Eisenmenger Syndrome; Neurocytoma; I Cell Disease; Schwannomatosis; Gliosarcoma; Platybasia; Lymphoma, T-Cell; Tooth Resorption; Anaplastic Astrocytoma; Carcinoma, Embryonal; Decompression Sickness; Familial Partial Lipodystrophy; Palatal Neoplasms; Langerhans Cell Histiocytosis; Sprains and Strains; Hereditary Breast and Ovarian Cancer Syndrome; Pulmonary Atresia; Mucopolysaccharidosis II; Vestibular Diseases; 46, XX Disorders of Sex Development; Back Pain; Ciguatera Fish Poisoning; Elbow Tendinopathy; Neurodegeneration With Brain Iron Accumulation; Iatrogenic Disease; Sciatic Neuropathy; Gliosis; Pleomorphic Xanthoastrocytoma; Lymphoma, T-Cell, Cutaneous; Tooth Wear; Anaplastic Ependymoma; Carcinoma, Endometrioid; Deficiency Diseases; Familial Partial Lipodystrophy Type 2; Palatopharyngeal Incompetence; Language Development Disorders; Sprue, Tropical; Hereditary Central Nervous System Demyelinating Diseases; Pulmonary Atresia With Ventricular Septal Defect; Mucopolysaccharidosis III; Vestibular Neuronitis; 47 XXX Syndrome; Bacteremia; Ciguatera Poisoning; Elective Mutism; Neurodegenerative Diseases; IBMPFD; Sciatica; Glomerulonephritis; Pleural Diseases; Lymphoma, T-Cell, Peripheral; Tooth, Impacted; Anaplastic Ganglioglioma; Carcinoma, Giant Cell; Deficiency of Interleukin-1 Receptor Antagonist; Familial Primary Pulmonary Hypertension; Palindromic Rheumatism; Language Disorders; Squamous Cell Carcinoma of Head and Neck; Hereditary Cerebral Hemorrhage With Amyloidosis; Pulmonary Blastoma; Mucopolysaccharidosis IV; Vestibulocochlear Nerve Diseases; 47, XYY Syndrome; Bacterial Infections; Ciliary Motility Disorders; Elephantiasis; Neurodermatitis; Ichthyosiform Erythroderma, Congenital; Scimitar Syndrome; Glomerulonephritis, IGA; Pleural Effusion; Lymphomatoid Granulomatosis; Tooth, Nonvital; Anaplastic Large Cell Lymphoma; Carcinoma, Hepatocellular; Deficiency of N-glycanase 1; Familial Prostate Cancer; Pallister-Hall Syndrome; Large Granular Lymphocyte Leukemia; Squamous Intraepithelial Lesions of the Cervix; Hereditary Coproporphyria; Pulmonary Disease, Chronic Obstructive; Mucopolysaccharidosis Type I; Vibrio Infections; 5-Nucleotidase Syndrome; Bacterial Meningitis; Ciliopathies; Elephantiasis, Filarial; Neurodevelopmental Disorders; Ichthyosis; Scleredema; Glomerulonephritis, Membranoproliferative; Pleural Effusion, Malignant; Lymphomatoid Papulosis; Tooth, Supernumerary; Anaplastic Oligoastrocytoma; Carcinoma, Intraductal, Noninfiltrating; Degloving Injuries; Familial Thoracic Aortic Aneurysm and Dissection; Palmoplantar Keratoderma; Laron Syndrome; ST Elevation Myocardial Infarction; Hereditary Diffuse Gastric Cancer; Pulmonary Edema; Mucopolysaccharidosis Type II; Vipoma; 5q-Syndrome; Bacteriuria; Citrullinemia; Elliptocytosis, Hereditary; Neuroectodermal Tumors; Ichthyosis Linearis Circumflexa; Scleredema Adultorum; Glomerulonephritis, Membranous; Pleural Neoplasms; Lymphomatous Thyroiditis; Toothache; Anaplastic Oligodendroglioma; Carcinoma, Islet Cell; Deglutition Disorders; Familial Transthyretin Amyloidosis; Pancoast Syndrome; Larva Migrans; Staghorn Calculi; Hereditary Elliptocytosis; Pulmonary Edema of Mountaineers; Mucopolysaccharidosis Type III; Viral Hemorrhagic Fever; Abdomen, Acute; Bacteroides Infections; Citrullinemia Type I; Ellis-Van Creveld Syndrome; Neuroectodermal Tumors, Primitive; Ichthyosis Vulgaris; Scleritis; Glomerulosclerosis, Focal Segmental; Pleurisy; Lymphopenia; Torsades de Pointes; Anaplastic Pleomorphic Xanthoastrocytoma; Carcinoma, Large Cell; Dehydration; Familial Tumoral Calcinosis; Pancreatic Adenoma; Laryngeal Cancer; Staphylococcal Infections; Hereditary Fructose Intolerance; Pulmonary Embolism; Mucopolysaccharidosis Type IIIA; Viremia; Abdominal Abscess; Baetz-Greenwalt Syndrome; Classic Galactosemia; Emaciation; Neuroectodermal Tumors, Primitive, Peripheral; Ichthyosis, Lamellar; Scleroderma, Diffuse; Glomus Tumor; Pleuroparenchymal Fibroelastosis; Lymphoproliferative Disorders; Torsion Abnormality; Anaplastic Thyroid Cancer; Carcinoma, Lobular; Delayed Emergence from Anesthesia; Fanconi Anemia; Pancreatic Cancer; Laryngeal Cleft; Staphylococcal Skin Infections; Hereditary Hemorrhagic Telangiectasia; Pulmonary Emphysema; Mucopolysaccharidosis Type IIIB; Virilism; Abdominal Aortic Aneurysm; Bainbridge-Ropers Syndrome; Clear Cell Renal Cell Carcinoma; Emanuel Syndrome; Neuroendocrine Tumors; Ichthyosis, X-Linked; Scleroderma, Limited; Glossitis; Pleuropneumonia; Lymphosarcoma; Torticollis; Anastomotic Leak; Carcinoma, Medullary; Delayed Graft Function; Fanconi Syndrome; Pancreatic Cyst; Laryngeal Diseases; Staphylococcal Toxic Shock Syndrome; Hereditary Leiomyomatosis and Renal Cell Cancer; Pulmonary Eosinophilia; Mucopolysaccharidosis Type IV; Virus Associated Hemophagocytic Syndrome; Abdominal Injuries; Balanitis; Cleft Lip; Embolism; Neuroepithelioma; Idiopathic Achalasia; Scleroderma, Localized; Glossitis, Benign Migratory; Pleuropulmonary Blastoma; Lysosomal Acid Lipase Deficiency; Tourette Syndrome; ANCA-associated Vasculitis; Carcinoma, Merkel Cell; Delirium; Farber Lipogranulomatosis; Pancreatic Diseases; Laryngeal Edema; Stargardt Disease; Hereditary Methemoglobinemia; Pulmonary Fibrosis; Mucopolysaccharidosis Type IVA; Virus Diseases; Abdominal Neoplasms; Balanitis Xerotica Obliterans; Cleft Palate; Embolism and Thrombosis; Neurofibroma; Idiopathic CD4 Positive T-lymphocytopenia; Scleroderma, Systemic; Glossopharyngeal Nerve Diseases; PMM2-CDG (CDG-la); Lysosomal Storage Diseases; Toxemia; Ancylostomiasis; Carcinoma, Mucoepidermoid; Delusions; Farber's Disease; Pancreatic Fistula; Laryngeal Neoplasms; Starvation; Hereditary Motor and Sensory Neuropathy; Pulmonary Heart Disease; Mucopolysaccharidosis Type VI; Visceral Pain; Abdominal Obesity Metabolic Syndrome; Balkan Endemic Nephropathy; Clinical Deterioration; Embolism, Air; Neurofibroma, Plexiform; Idiopathic Hypersomnia; Sclerosing Mesenteritis; Glossopharyngeal Neuralgia; Pneumocephalus; Machado-Joseph Disease; Toxocariasis; Andersen Syndrome; Carcinoma, Neuroendocrine; Dementia; Fasciculation; Pancreatic Intraductal Neoplasms; Laryngeal Nerve Injuries; Status Asthmaticus; Hereditary Multiple Osteochondromas; Pulmonary Infarction; Mucopolysaccharidosis Type VII; Visceral Prolapse; Abdominal Pain; Balkan Nephropathy; CLIPPERS; Embolism, Cholesterol; Neurofibromatoses; Idiopathic Inflammatory Myopathy; Sclerosis; Glucagonoma; Pneumococcal Infections; Macroglossia; Toxoplasmosis; Andersen-Tawil Syndrome; Carcinoma, Non-Small-Cell Lung; Dementia, Multi-Infarct; Fasciitis; Pancreatic Neoplasms; Laryngeal Papillomatosis; Status Epilepticus; Hereditary Neuropathy With Liability to Pressure Palsies; Pulmonary Surfactant Protein B, Deficiency of; Mucopolysaccharidosis VI; Visceral Steatosis; Abducens Nerve Diseases; Bankart Lesions; Cloacal Exstrophy; Embolism, Fat; Neurofibromatosis 1; Idiopathic Interstitial Pneumonias; Scoliosis; Glucocorticoid Resistance; Pneumoconiosis; Macrophage Activation Syndrome; Toxoplasmosis, Cerebral; Anemia; Carcinoma, Ovarian Epithelial; Dementia, Vascular; Fasciitis, Necrotizing; Pancreatic Neuroendocrine Tumor; Laryngismus; Steatitis; Hereditary Pancreatitis; Pulmonary Valve Insufficiency; Mucopolysaccharidosis VII; Vision Disorders; Aberrant Crypt Foci; BAP1 Tumor Predisposition Syndrome; Clostridium Infections; Embolism, Paradoxical; Neurofibromatosis 2; Idiopathic Intracranial Hypertension; Scorpion Stings; Glucose Intolerance; Pneumocystis Infections; Macular Degeneration; Toxoplasmosis, Congenital; Anemia, Aplastic; Carcinoma, Pancreatic Ductal; Demodicidosis; Fasciitis, Plantar; Pancreatic Pseudocyst; Laryngitis; Steatorrhea; Hereditary Paraganglioma-pheochromocytoma; Pulmonary Valve Stenosis; Mucormycosis; Vision, Low; Abetalipoproteinemia; Bardet-Biedl Syndrome; CLOVES Syndrome; Embryo Loss; Neurofibromatosis Type 1; Idiopathic Pulmonary Fibrosis; Scotoma; Glucose Metabolism Disorders; Pneumocystis jirovecii Pneumonia; Macular Edema; Toxoplasmosis, Ocular; Anemia, Diamond-Blackfan; Carcinoma, Papillary; Demyelinating Autoimmune Diseases, CNS; Fascioliasis; Pancreatitis; Laryngomalacia; Stenosis, Pulmonary Artery; Hereditary Sensory and Autonomic Neuropathies; Pulmonary Vein Stenosis; Mucositis; Vitamin A Deficiency; Abnormal Karyotype; Bardet-Biedl Syndrome 1; Clubfoot; Embryonal Carcinoma; Neurofibromatosis Type 2; Idiopathic Pulmonary Hemosiderosis; Scott Syndrome; Glucose Transporter Type 1 Deficiency Syndrome; Pneumocystosis; Magnesium Deficiency; Tracheal Diseases; Anemia, Dyserythropoietic, Congenital; Carcinoma, Papillary, Follicular; Demyelinating Diseases; Fat Necrosis; Pancreatitis, Acute Necrotizing; Laryngopharyngeal Reflux; Stenosis, Pulmonary Vein; Hereditary Sensory and Motor Neuropathy; Pulmonary Veno-Occlusive Disease; Muenke Syndrome; Vitamin B 12 Deficiency; Abnormalities, Drug-Induced; Bardet-Biedl Syndrome 10; Cluster Headache; Embryonal Sarcoma; Neurofibrosarcoma; Idiopathic Subglottic Tracheal Stenosis; Scrub Typhus; Glucose-6-phosphate Dehydrogenase Deficiency; Pneumonia; Mal De Debarquement Syndrome; Tracheal Neoplasms; Anemia, Hemolytic; Carcinoma, Renal Cell; Dendritic Cell Sarcoma, Follicular; Fatal Familial Insomnia; Pancreatitis, Alcoholic; Laryngostenosis; Stenotrophomonas maltophilia Infection; Hereditary Sensory Neuropathy Type 1; Pulmonic Stenosis; Muir-Torre Syndrome; Vitamin B 6 Deficiency; Abnormalities, Multiple; Bardet-Biedl Syndrome 11; Coagulation Protein Disorders; Embryonal Tumor With Multilayered Rosettes; Neurogenic Bowel; Idiopathic Thrombocytopeniaurpura; Scurvy; Glucose-galactose Malabsorption; Pneumonia, Aspiration; Malabsorption Syndromes; Tracheal Stenosis; Anemia, Hemolytic, Autoimmune; Carcinoma, Signet Ring Cell; Dendritic Cell Sarcoma, Interdigitating; Fatigue; Pancreatitis, Chronic; Lassa Fever; Stereotypic Movement Disorder; Hereditary Spastic Paraplegia; Pulpitis; Mullerian Aplasia; Vitamin D Deficiency; Abnormalities, Radiation-Induced; Bardet-Biedl Syndrome 12; Coal Worker's Pneumoconiosis; Emergence Delirium; Neurogenic Inflammation; IgA Deficiency; Seasonal Affective Disorder; Glucosephosphate Dehydrogenase Deficiency; Pneumonia, Bacterial; Malaria; Tracheitis; Anemia, Hemolytic, Congenital; Carcinoma, Skin Appendage; Dendritic Cell Tumor; Fatigue Syndrome, Chronic; Pancreatitis, Graft; Late Onset Disorders; Steroid Metabolism, Inborn Errors; Hereditary Spherocytosis; Punctate Inner Choroidopathy; Multicentric Castleman Disease; Vitamin E Deficiency; Abortion, Habitual; Bardet-Biedl Syndrome 2; Coats Disease; Emergencies; Neuroleptic Malignant Syndrome; IgA Nephropathy; Sebaceous Gland Diseases; Glutaric Acidemia Type II; Pneumonia, Eosinophilic; Malaria, Cerebral; Tracheobronchomalacia; Anemia, Hemolytic, Congenital Nonspherocytic; Carcinoma, Small Cell; Dengue; Fatty Liver; Pancytopenia; Late-Onset Familial Alzheimer Disease; Stevens-Johnson Syndrome; Heredodegenerative Disorders, Nervous System; Pupil Disorders; Multicentric Reticulohistiocytosis; Vitamin K Deficiency; Abortion, Incomplete; Bare Lymphocyte Syndrome; Cocaine Antenatal Exposure; Emery-Dreifuss Muscular Dystrophy; Neurologic Manifestations; IgG Deficiency; Seckel Syndrome; Glutathione Synthetase Deficiency; Pneumonia, Mycoplasma; Malaria, Falciparum; Tracheobronchomegaly; Anemia, Hypochromic; Carcinoma, Squamous Cell; Dengue Fever; Fatty Liver, Alcoholic; PANDAS; Late-onset Retinal Degeneration; Stevens-Johnson Syndrome/toxic Epidermal Necrolysis; Hermanski-Pudlak Syndrome; Pure Autonomic Failure; Multicore Disease; Vitelliform Macular Dystrophy; Abortion, Missed; Bare Lymphocyte Syndrome 2; Cocaine-Related Disorders; Emphysema; Neuroma; IgG4-related Disease; Secondary Adrenal Insufficiency; Glycogen Storage Disease; Pneumonia, Necrotizing; Malaria, Vivax; Tracheoesophageal Fistula; Anemia, Hypoplastic, Congenital; Carcinoma, Transitional Cell; Dense Deposit Disease; Favism; Panic Disorder; Latent Autoimmune Diabetes in Adults; Stickler Syndrome; Hermansky-Pudlak Syndrome; Pure Red Cell Aplasia; Multicystic Dysplastic Kidney; Vitiligo; Abortion, Septic; Baroreflex Failure; Coccidioidomycosis; Empyema; Neuroma, Acoustic; Ileal Diseases; Seizures; Glycogen Storage Disease Type 2; Pneumonia, Pneumococcal; Malformations of Cortical Development; Tracheomalacia; Anemia, Iron-Deficiency; Carcinoma, Verrucous; Dent Disease; Febrile Neutropenia; Panniculitis; Latent Tuberculosis; Stickler Syndrome Type 1; Hernia; Purine Nucleoside Phosphorylase Deficiency; Multicystic Renal Dysplasia, Bilateral; Vitreoretinopathy, Proliferative; Abortion, Spontaneous; Barotrauma; Coccidiosis; Empyema, Pleural; Neuromuscular Diseases; Ileitis; Seizures, Febrile; Glycogen Storage Disease Type 3; Pneumonia, Pneumocystis; Malformations of Cortical Development, Group II; Trachoma; Anemia, Megaloblastic; Carcinosarcoma; Dental Calculus; Fecal Impaction; Panniculitis, Peritoneal; Lateral Sinus Thrombosis; Stickler Syndrome, Type 2; Hernia, Abdominal; Purine-Pyrimidine Metabolism, Inborn Errors; Multifocal Choroiditis; Vitreous Detachment; Abortion, Threatened; Barrett Esophagus; Coccygodynia; Encephalitis; Neuromuscular Junction Diseases; Ileus; Self-Mutilation; Glycogen Storage Disease Type 4; Pneumonia, Staphylococcal; Malignant Carcinoid Syndrome; Transfusion Reaction; Anemia, Neonatal; Cardiac Complexes, Premature; Dental Caries; Fecal Incontinence; Pantothenate Kinase-Associated Neurodegeneration; Latex Hypersensitivity; Stiff-Person Syndrome; Hernia, Diaphragmatic; Purpura; Multifocal Fibrosclerosis; Vitreous Hemorrhage; Abruptio Placentae; Barth Syndrome; Cochlear Diseases; Encephalitis, Herpes Simplex; Neuromuscular Manifestations; Iliac Aneurysm; Self-Injurious Behavior; Glycogen Storage Disease Type 5; Pneumonia, Ventilator-Associated; Malignant Germ Cell Tumor; Transfusion-Related Acute Lung Injury; Anemia, Pernicious; Cardiac Conduction System Disease; Dental Deposits; Feeding and Eating Disorders; Panuveitis; Laurence-Moon Syndrome; Still's Disease, Adult-Onset; Hernia, Femoral; Purpura Fulminans; Multifocal Lymphangioendotheliomatosis With Thrombocytopenia; VLCAD Deficiency; Abscess; Bartonella Infections; Cockayne Syndrome; Encephalitis, Japanese; Neuromyelitis Optica; Iliotibial Band Syndrome; Semantic Dementia; Glycogen Storage Disease Type 7; Pneumonia, Viral; Malignant Hyperthermia; Transient Global Amnesia; Anemia, Refractory; Cardiac Output, High; Dental Enamel Hypoplasia; Feeding and Eating Disorders of Childhood; Papillary Glioneuronal Tumors; LCAD Deficiency; Stillbirth; Hernia, Hiatal; Purpura, Schoenlein-Henoch; Multifocal Motor Neuropathy; Vocal Cord Dysfunction; Absent Patella; Bartter Syndrome; Cogan Syndrome; Encephalitis, Tick-Borne; Neuromyelitis Optica Spectrum Disorder; Illness Behavior; Seminoma; Glycogen Storage Disease Type I; Pneumoperitoneum; Malignant Mesenchymoma; Transient Myeloproliferative Syndrome; Anemia, Refractory, with Excess of Blasts; Cardiac Output, Low; Dental Fissures; Felty Syndrome; Papillary Renal Cell Carcinoma; LCHAD Deficiency; Stomach Cancer; Hernia, Inguinal; Purpura, Thrombocytopenia Multiple Acyl Coenzyme A Dehydrogenase Deficiency; Vocal Cord Paralysis; Acalculous Cholecystitis; Basal Cell Carcinoma, Multiple; Cognition Disorders; Encephalitis, Viral; Neuronal Ceroid Lipofuscinosis 2; Illusions; Sensation Disorders; Glycogen Storage Disease Type II; Pneumothorax; Malignant Mesothelioma; Transient Tachypnea of the Newborn; Anemia, Sickle Cell; Cardiac Rupture; Dental Leakage; Female Athlete Triad Syndrome; Papillary Thyroid Carcinoma; Lead Poisoning; Stomach Diseases; Hernia, Umbilical; Purpura, Thrombocytopeniaiopathic; Multiple Chemical Sensitivity; Vogt-Koyanagi-Harada Disease; Acanthamoeba Keratitis; Basal Cell Nevus Syndrome; Cognitive Dysfunction; Encephalocele; Neuronal Ceroid Lipofuscinosis 3; Immune Complex Diseases; SEPN1-related Myopathy; Glycogen Storage Disease Type IIb; POEMS Syndrome; Malignant Migrating Partial Seizures of Infancy; Transitional Cell Cancer of the Renal Pelvis and Ureter; Anencephaly; Cardiac Tamponade; Dental Occlusion, Traumatic; Feminization; Papilledema; Learning Disorders; Stomach Neoplasms; Hernia, Ventral; Purpura, Thrombotic Thrombocytopeniaultiple Chronic Conditions; Voice Disorders; Acanthocheilonemiasis; Basal Ganglia Diseases; Cohen Syndrome; Encephalomalacia; Neuronal Ceroid-Lipofuscinoses; Immune Reconstitution Inflammatory Syndrome; Sepsis; Glycogen Storage Disease Type III; Poisoning; Malignant Mixed Mullerian Tumor; Transitional Cell Carcinoma; Anetoderma; Cardio-Renal Syndrome; Dental Plaque; Femoracetabular Impingement; Papilloma; Leber Congenital Amaurosis; Stomach Ulcer; Hernias, Diaphragmatic, Congenital; Pustular Psoriasis; Multiple Endocrine Neoplasia; Vomiting; Acanthosis Nigricans; Basal Ganglia Hemorrhage; Coinfection; Encephalomyelitis; Neuronal Intranuclear Inclusion Disease; Immune System Diseases; Sepsis-Associated Encephalopathy; Glycogen Storage Disease Type IV; Poliomyelitis; Malignant Peripheral Nerve Sheath Tumor; Translocation, Genetic; Aneuploidy; Cardiomegaly; Dental Pulp Diseases; Femoral Fractures; Papilloma, Intraductal; Leber Congenital Amaurosis 10; Stomatitis; Heroin Dependence; Pustulosis Palmaris Et Plantaris; Multiple Endocrine Neoplasia Type 1; Von Hippel-Lindau Disease; Accessory Atrioventricular Bundle; Becker Muscular Dystrophy; Cold Agglutinin Disease; Encephalomyelitis, Acute Disseminated; Neurosyphilis; Immune Thrombocytopenia; Septo-Optic Dysplasia; Glycogen Storage Disease Type V; Polyarteritis Nodosa; Malingering; Transposition of Great Vessels; Aneurysm; Cardiomyopathies; Dental Pulp Exposure; Femoral Neck Fractures; Papilloma, Inverted; Leber Hereditary Optic Neuropathy; Stomatitis, Aphthous; Herpangina; Pyelectasis; Multiple Endocrine Neoplasia Type 2; Von Willebrand Disease, Type 1; Accessory Nerve Diseases; Beckwith-Wiedemann Syndrome; Cold Urticaria; Encephalomyelitis, Eastern Equine; Neurotic Disorders; Immunoblastic Lymphadenopathy; Septo-optic Dysplasia Spectrum; Glycogen Storage Disease Type VI; Polyarticular Onset Juvenile Idiopathic Arthritis; Malnutrition; Transposition of the Great Arteries; Aneurysm, Dissecting; Cardiomyopathy Due to Anthracyclines; Dental Pulp Necrosis; Femoral Neuropathy; Papillomavirus Infections; Lecithin Cholesterol Acyltransferase Deficiency; Stomatitis, Denture; Herpes Genitalis; Pyelonephritis; Multiple Endocrine Neoplasia Type 2a; Von Willebrand Disease, Type 2; Accessory Nerve Injuries; BehÃ § et Disease; Colic; Encephalomyelitis, Equine; Neurotoxicity Syndromes; Immunodeficiency With Hyper IgM Type 1; Seroma; Glycogen Storage Disease Type VII; Polychondritis, Relapsing; Malocclusion; Transverse Myelitis; Aneurysm, False; Cardiomyopathy, Alcoholic; Dentatorubral-pallidoluysian Atrophy; Femur Head Necrosis; Papillon Lefevre Syndrome; Ledderhose Disease; Stomatitis, Herpetic; Herpes Labialis; Pyloric Stenosis; Multiple Endocrine Neoplasia Type 2b; Von Willebrand Disease, Type 3; Achlorhydria; Behavior, Addictive; Colitis; Encephalomyelitis, Venezuelan Equine; Neutral Lipid Storage Disease With Myopathy; Immunoglobulin G4-Related Disease; Serotonin Syndrome; Glycogen Storage Disease Type VIII; Polycystic Kidney Diseases; Malocclusion, Angle Class I; Trauma and Stressor Related Disorders; Aneurysm, Ruptured; Cardiomyopathy, Dilated; Dentin Sensitivity; Fetal Alcohol Spectrum Disorders; Papillon-Lefevre Disease; Left Ventricular Noncompaction; Stomatognathic Diseases; Herpes Simplex; Pyloric Stenosis, Hypertrophic; Multiple Mitochondrial Dysfunctions Syndrome; Von Willebrand Diseases; Achondroplasia; Behavioral Symptoms; Colitis, Collagenous; Encephalomyelitis, Western Equine; Neutropenia; Immunoglobulin Light-chain Amyloidosis; Serpiginous Choroiditis; Glycosuria, Renal; Polycystic Kidney, Autosomal Dominant; Malocclusion, Angle Class II; Trauma, Nervous System; Aneurysmal Bone Cysts; Cardiomyopathy, Hypertrophic; Dentin, Secondary; Fetal and Neonatal Alloimmune Thrombocytopenia; Paraganglioma; Leg Injuries; Stomatognathic System Abnormalities; Herpes Simplex Encephalitis; Pyoderma; Multiple Myeloma; Vulvar Cancer; Acid-Base Imbalance; Behavioral Variant of Frontotemporal Dementia; Colitis, Ischemic; Enchondroma; Nevi and Melanomas; Immunologic Deficiency Syndromes; Sertoli Cell Tumor; GM1 Gangliosidosis; Polycystic Kidney, Autosomal Recessive; Malocclusion, Angle Class III; Trematode Infections; Angelman Syndrome; Cardiomyopathy, Hypertrophic, Familial; Dentinogenesis Imperfecta; Fetal Death; Paraganglioma, Extra-Adrenal; Leg Length Inequality; Strabismus; Herpes Zoster; Pyoderma Gangrenosum; Multiple Organ Failure; Vulvar Diseases; Acidosis; Behcet Syndrome; Colitis, Lymphocytic; Encopresis; Nevoid Basal Cell Carcinoma Syndrome; Immunoproliferative Disorders; Sertoli Cell-Only Syndrome; GM3 Synthase Deficiency; Polycystic Liver Disease; Mammary Neoplasms, Animal; Tremor; Angina Pectoris; Cardiomyopathy, Restrictive; Dentofacial Deformities; Fetal Diseases; Paragangliomas 1; Leg Ulcer; Streptococcal Group A Invasive Disease; Herpes Zoster Ophthalmicus; Pyomyositis; Multiple Pulmonary Nodules; Vulvar Lichen Sclerosus; Acidosis, Lactic; Bell Palsy; Colitis, Microscopic; End Stage Liver Disease; Nevus; Impairment of Oral Perception; Sertoli-Leydig Cell Tumor; Goiter; Polycystic Ovary Syndrome; Mandibular Diseases; Tremor Hereditary Essential, 1; Angina Pectoris, Variant; Cardiospasm; Denys-Drash Syndrome; Fetal Distress; Parainfluenza Virus Type 3; Legg-Calve-Perthes Disease; Streptococcal Infections; Herpes Zoster Oticus; Pyonephrosis; Multiple Sclerosis; Vulvar Neoplasms; Acidosis, Renal Tubular; Benign Autosomal Dominant Myopathy; Colitis, Ulcerative; Endemic Kaposi Sarcoma; Nevus of Ota; Imperforate Anus; Severe Acute Malnutrition; Goiter, Nodular; Polycythemia; Mandibular Fractures; Treponema Infection; Angina, Stable; Cardiotoxicity; Dependent Personality Disorder; Fetal Edema; Paralysis; Legionellosis; Stress Disorders, Post-Traumatic; Herpesviridae Infections; Pyridoxine Deficiency; Multiple Sclerosis, Chronic Progressive; Vulvar Vestibulitis; Acidosis, Respiratory; Benign Essential Blepharospasm; Collagen Diseases; Endocardial Cushion Defects; Nevus, Blue; Impetigo; Severe Acute Respiratory Syndrome; Goldenhar Disease; Polycythemia Vera; Mandibular Injuries; Treponemal Infections; Angina, Unstable; Cardiovascular Abnormalities; Depersonalization; Fetal Growth Retardation; Paralysis, Hyperkalemic Periodic; Legionnairesâ€™ Disease; Stress Disorders, Traumatic; Heterotaxy; Pyridoxine-dependent Epilepsy; Multiple Sclerosis, Relapsing-Remitting; Vulvar Vestibulitis Syndrome; Acinetobacter Infections; Benign Paroxysmal Positional Vertigo; Collagenous Sprue; Endocarditis; Nevus, Epithelioid and Spindle Cell; Implant Capsular Contracture; Severe Combined Immunodeficiency; Goldenhar Syndrome; Polydactyly; Mandibular Neoplasms; Trichiasis; Angiodysplasia; Cardiovascular Infections; Depersonalization/derealization Disorder; Fetal Hypoxia; Paralysis, Obstetric; Legionnaires' Disease; Stress Disorders, Traumatic, Acute; Heterotaxy Syndrome; Pyropoikilocytosis Hereditary; Multiple Sulfatase Deficiency; Vulvodynia; Acinic Cell Carcinoma; Benign Rolandic Epilepsy (BRE); Collecting Duct Carcinoma; Endocarditis, Bacterial; Nevus, Pigmented; Impotence, Vasculogenic; Severe Combined Immunodeficiency, Atypical; Goldmann-Favre Syndrome; Polydipsia; Mannosidase Deficiency Diseases; Trichomonas Infections; Angioedema; Carney Complex; Depression; Fetal Macrosomia; Paramyotonia Congenita; Legius Syndrome; Stress, Psychological; Hiccup; Pyruvate Carboxylase Deficiency; Multiple Sulfatase Deficiency Disease; Vulvovaginitis; Acne Keloid; Beriberi; Colles' Fracture; Endocarditis, Subacute Bacterial; Nevus, Sebaceous of Jadassohn; Impulsive Behavior; Severe Congenital Neutropenia; Gonadal Disorders; Polydipsia, Psychogenic; Mannosidosis, Beta A, Lysosomal; Trichomonas Vaginitis; Angioedemas, Hereditary; Carnitine Palmitoyltransferase 2 Deficiency; Depression, Postpartum; Fetal Membranes, Premature Rupture; Paramyxoviridae Infections; Leigh Disease; Striae Distensae; Hidradenitis; Pyruvate Carboxylase Deficiency Disease; Multiple System Atrophy; Waardenburg Syndrome; Acne Vulgaris; Berylliosis; Coloboma; Endocrine Gland Neoplasms; New Daily-persistent Headache; Inappropriate ADH Syndrome; Severe Dengue; Gonadal Dysgenesis; Polyembryoma; Mansonelliasis; Trichothiodystrophy; Angiofibroma; Caroli Disease; Depressive Disorder; Fetal Nutrition Disorders; Paranasal Sinus Diseases; Leigh Syndrome; Stroke; Hidradenitis Suppurativa; Pyruvate Decarboxylase Deficiency; Multiple Trauma; WAGR Syndrome; Acoustic Neuroma; Beryllium Disease; Coloboma of Optic Nerve; Endocrine System Diseases; Newcastle Disease; Inborn Amino Acid Metabolism Disorder; Sex Chromosome Aberrations; Gonococcal Conjunctivitis; Polyendocrinopathies, Autoimmune; Mantle Cell Lymphoma; Trichothiodystrophy Syndromes; Angioid Streaks; Carotid Artery Diseases; Depressive Disorder, Major; Fetal Thalidomide Syndrome; Paranasal Sinus Neoplasms; Leiner Disease; Stroke, Lacunar; Hidradenocarcinoma; Pyruvate Dehydrogenase Complex Deficiency; Mumps; Waldenstrom Macroglobulinemia; Acquired Hemophilia; Best Vitelliform Macular Dystrophy; Colonic Diseases; Endodermal Sinus Tumor; Niemann-Pick Disease Type A; Incisional Hernia; Sex Chromosome Disorders; Gonorrhea; Polyhydramnios; Maple Syrup Urine Disease; Trichotillomania; Angioimmunoblastic Lymphadenopathy With Dysproteinemia; Carotid Artery Injuries; Depressive Disorder, Treatment-Resistant; Fetal Weight; Paraneoplastic Polyneuropathy; Leiomyoma; Strongyloidiasis; Hip Dislocation; Pyruvate Dehydrogenase Complex Deficiency Disease; Muscle Cramp; Walker-Warburg Syndrome; Acquired Hemophilia A; Beta-galactosidase-1 Deficiency; Colonic Diseases, Functional; Endoleak; Niemann-Pick Disease Type B; Inclusion Body Myopathy 2; Sex Cord-Gonadal Stromal Tumors; Goodpasture Syndrome; Polymicrogyria; Marburg Hemorrhagic Fever; Trichuriasis; Angioimmunoblastic T-cell Lymphoma; Carotid Artery Thrombosis; Dermatitis; Fetofetal Transfusion; Paraneoplastic Syndromes; Leiomyomatosis; Struma Ovarii; Hip Dislocation, Congenital; Pyruvate Kinase Deficiency; Muscle Eye Brain Disease; Wandering Behavior; Acquired Hyperostosis Syndrome; beta-Mannosidosis; Colonic Neoplasms; Endolymphatic Hydrops; Niemann-Pick Disease Type C1; Inclusion Body Myositis; Sexual Dysfunction, Physiological; Gordon Syndrome; Polymorphic Reticulosis; Marburg Virus Disease; Tricuspid Atresia; Angiokeratoma; Carotid Body Tumor; Dermatitis Herpetiformis; Fetomaternal Transfusion; Paraneoplastic Syndromes, Nervous System; Leiomyosarcoma; Stupor; Hip Fractures; Pyruvate Metabolism, Inborn Errors; Muscle Hypertonia; War-Related Injuries; Acquired Immunodeficiency Syndrome; Beta-Propeller Protein-Associated Neurodegeneration; Colonic Polyps; Endolymphatic Sac Tumor; Niemann-Pick Disease, Type A; Inclusion Conjunctivitis; Sexual Dysfunctions, Psychological; Gorham's Disease; Polymyalgia Rheumatica; Marcus Gunn Phenomenon; Tricuspid Valve Insufficiency; Angiolipoma; Carotid Stenosis; Dermatitis, Allergic Contact; Fever; Paranoid Disorders; Leishmaniasis; Sturge-Weber Syndrome; Hip Injuries; Pyuria; Muscle Hypotonia; Warm Antibody Hemolytic Anemia; Acquired Von Willebrand Syndrome; beta-Thalassemia; Colonic Pseudo-Obstruction; Endometrial Hyperplasia; Niemann-Pick Disease, Type B; Incontinentia Pigmenti; Sexually Transmitted Diseases; Gout; Polymyositis; Marfan Syndrome; Trigeminal Autonomic Cephalalgias; Angiolymphoid Hyperplasia with Eosinophilia; Carotid-Cavernous Sinus Fistula; Dermatitis, Atopic; Fever of Unknown Origin; Paraparesis; Leishmaniasis, Cutaneous; Stuttering; Hirschsprung Disease; Q Fever; Muscle Neoplasms; Warts; Acral Lentiginous Melanoma; Bethlem Myopathy; Color Vision Defects; Endometrial Neoplasms; Niemann-Pick Disease, Type C; Indolent B Cell Lymphoma; Sexually Transmitted Diseases, Bacterial; Graft Occlusion, Vascular; Polyneuropathies; Marginal Zone Lymphoma; Trigeminal Nerve Diseases; Angiomatosis; Carpal Tunnel Syndrome; Dermatitis, Contact; Fibrillary Glomerulonephritis; Paraparesis, Spastic; Leishmaniasis, Mucocutaneous; Subarachnoid Hemorrhage; Hirsutism; Quadriplegia; Muscle Rigidity; Wasting Disease, Chronic; Acrodermatitis; Biemond Syndrome; Colorectal Neoplasms; Endometrial Stromal Sarcoma; Niemann-Pick Diseases; Inert Gas Narcosis; Sexually Transmitted Diseases, Viral; Graft vs Host Disease; Polyomavirus Allograft Nephropathy; Marijuana Abuse; Trigeminal Nerve Injuries; Angiomyolipoma; Carpenter Syndrome; Dermatitis, Exfoliative; Fibrinogen Deficiency, Congenital; Paraparesis, Tropical Spastic; Leishmaniasis, Visceral; Subarachnoid Hemorrhage, Traumatic; His Bundle Tachycardia; Quality of Life; Muscle Spasticity; Wasting Syndrome; Acrodermatitis Enteropathica; Bilateral Vestibulopathy; Colorectal Neoplasms, Hereditary Nonpolyposis; Endometrial Stromal Tumors; Night Blindness; Infant Death; Sezary Syndrome; Gram-Negative Bacterial Infections; Polyomavirus Infections; Marijuana Use; Trigeminal Neuralgia; Angiosarcoma of the Liver; Cartilage Diseases; Dermatitis, Irritant; Fibroadenoma; Paraphilic Disorders; Lennox Gastaut Syndrome; Subclavian Steal Syndrome; Histiocytic Disorders, Malignant; Quinquaud's Decalvans Folliculitis; Muscle Weakness; Water Intoxication; Acromegaly; Bile Duct Cancer; Coma; Endometriosis; Night Eating Syndrome; Infant Nutrition Disorders; Shaken Baby Syndrome; Gram-Positive Bacterial Infections; Polyps; Marinesco-Sjogren Syndrome; Trigger Finger Disorder; Angiostrongyliasis; Cartilage-hair Hypoplasia; Dermatitis, Occupational; Fibrocystic Breast Disease; Paraphimosis; Lens Diseases; Subcorneal Pustular Dermatosis; Histiocytic Necrotizing Lymphadenitis; Rabies; Muscular Atrophy; Water-Electrolyte Imbalance; ACTH Syndrome, Ectopic; Bile Duct Cysts; Coma, Post-Head Injury; Endometritis; No-Reflow Phenomenon; Infant, Newborn, Diseases; Shared Paranoid Disorder; Grand Kaine Fulling Syndrome; Polyradiculoneuropathy; Marshall Syndrome; Trigger Thumb; Anhedonia; Castleman Disease; Dermatitis, Perioral; Fibrodysplasia Ossificans Progressiva; Paraplegia; Lens Subluxation; Subcutaneous Emphysema; Histiocytic Sarcoma; Radial Neuropathy; Muscular Atrophy, Spinal; Waterborne Diseases; ACTH-independent Macronodular Adrenal Hyperplasia; Bile Duct Diseases; Combat Disorders; Endomyocardial Fibrosis; Nociceptive Pain; Infant, Premature, Diseases; Shashi-Pena Syndrome; Granular Cell Tumor; Polyradiculoneuropathy, Chronic Inflammatory Demyelinating; Masked Hypertension; Triple Negative Breast Neoplasms; Aniridia; Cat-Scratch Disease; Dermatitis, Photoallergic; Fibrolamellar Carcinoma; Paraproteinemias; Lentigo; Subcutaneous Panniculitis-like T-cell Lymphoma; Histiocytoma; Radiation Induced Brachial Plexopathy; Muscular Diseases; Watermelon Stomach; ACTH-Secreting Pituitary Adenoma; Bile Duct Neoplasms; Common Bile Duct Diseases; Endophthalmitis; Nocturia; Infantile Apnea; Sheldon-Hall Syndrome; Granulocytopenia; Polyradiculopathy; Mast Cell Activation Syndrome; Trismus; Aniridia-Ptosis-Intellectual Disability-Familial Obesity; Catalepsy; Dermatitis, Phototoxic; Fibroma; Parapsoriasis; Lentigo Maligna Melanoma; Subdural Effusion; Histiocytoma, Benign Fibrous; Radiation Injuries; Muscular Disorders, Atrophic; WDHA Syndrome; Actinic Cheilitis; Bile Reflux; Common Bile Duct Neoplasms; Endotoxemia; Nocturnal Enuresis; Infantile Myofibromatosis; Shellfish Hypersensitivity; Granuloma; Polyuria; Mast-Cell Sarcoma; Trisomy; Anisakiasis; Cataplexy; Dermatitis, Seborrheic; Fibromatosis, Abdominal; Parasitemia; Lentivirus Infections; Subependymal Giant Cell Astrocytoma; Histiocytoma, Malignant Fibrous; Radiation Injuries, Experimental; Muscular Dystrophies; Weber Syndrome; Activated Protein C Resistance; Biliary Atresia; Common Cold; Engraftment Syndrome; Nocturnal Myoclonus Syndrome; Infantile Neuroaxonal Dystrophy; Shigellosis; Granuloma Annulare; Popliteal Cyst; Mastitis; Trisomy 13; Aniseikonia; Cataract; Dermatofibrosarcoma; Fibromatosis, Aggressive; Parasitic Diseases; LEOPARD Syndrome; Subependymoma; Histiocytosis; Radiation Pneumonitis; Muscular Dystrophies, Limb-Girdle; Weight Gain; Acute Articular Rheumatism; Biliary Atresia Intrahepatic Syndromic Form; Common Variable Immunodeficiency; Enlarged Vestibular Aqueduct Syndrome; Nodding Syndrome; Infantile Onset Spinocerebellar Ataxia; Shock; Granuloma, Giant Cell; Porokeratosis; Mastocytoma; Trisomy 13 Syndrome; Anisometropia; Catastrophic Antiphospholipid Syndrome; Dermatofibrosarcoma Protuberans; Fibromatosis, Plantar; Parasomnias; Leprosy; Substance Abuse, Intravenous; Histiocytosis, Langerhans-Cell; Radiculopathy; Muscular Dystrophy, Duchenne; Weight Loss; Acute Chest Syndrome; Biliary Dyskinesia; Communicable Diseases; Enophthalmos; Nodular Melanoma; Infantile Scoliosis; Shock, Cardiogenic; Granuloma, Laryngeal; Porphyria Cutanea Tarda; Mastocytosis; Trisomy 18; Ankle Fractures; Catastrophic Illness; Dermatomycoses; Fibromuscular Dysplasia; Parathyroid Carcinoma; Leprosy, Lepromatous; Substance Withdrawal Syndrome; Histiocytosis, Non-Langerhans-Cell; Radiodermatitis; Muscular Dystrophy, Emery-Dreifuss; Weil Disease; Acute Coronary Syndrome; Biliary Fistula; Communicable Diseases, Emerging; Enteritis; Nodular Regenerative Hyperplasia; Infantile-onset Ascending Hereditary Spastic Paralysis; Shock, Hemorrhagic; Granuloma, Plasma Cell; Porphyria, Acute Intermittent; Mastocytosis, Cutaneous; Trisomy 18 Syndrome; Ankle Injuries; Catatonia; Dermatomyositis; Fibromyalgia; Parathyroid Diseases; Leprosy, Multibacillary; Substance-Related Disorders; Histiocytosis, Sinus; Radius Fractures; Muscular Dystrophy, Facioscapulohumeral; Wells Syndrome; Acute Disease; Biliary Tract Cancer; Communication Disorders; Enterobacteriaceae Infections; Non 24 Hour Sleep Wake Disorder; Infarction; Shock, Septic; Granulomatosis with Polyangiitis; Porphyria, Erythropoietic; Mastocytosis, Systemic; Trochlear Nerve Diseases; Ankyloglossia; Catecholaminergic Polymorphic Ventricular Tachycardia; Dermoid Cyst; Fibrosarcoma; Parathyroid Neoplasms; Leptospirosis; Succinic Semialdehyde Dehydrogenase Deficiency; Histoplasmosis; Rapid-onset Dystonia-parkinsonism; Muscular Dystrophy, Oculopharyngeal; Werner Syndrome; Acute Disseminated Encephalomyelitis; Biliary Tract Diseases; Community-Acquired Infections; Enterocolitis; Non Functioning Pancreatic Endocrine Tumor; Infarction, Anterior Cerebral Artery; Shock, Surgical; Granulomatous Disease, Chronic; Porphyria, Variegate; Mastodynia; Trophoblastic Neoplasms; Ankylosing Vertebral Hyperostosis With Tylosis; Catheter-Related Infections; Desmoid Tumor; Fibrosis; Paratuberculosis; Leriche Syndrome; Sudden Infant Death; HIV Enteropathy; Rare Diseases; Musculoskeletal Abnormalities; Wernicke Encephalopathy; Acute Erythroid Leukemia; Biliary Tract Neoplasms; Compartment Syndromes; Enterocolitis, Necrotizing; Non-alcoholic Fatty Liver Disease; Infarction, Middle Cerebral Artery; Shock, Traumatic; Granulomatous Mastitis; Porphyrias; Mastoiditis; Trophoblastic Tumor, Placental Site; Ankylosis; Cauda Equina Syndrome; Desmoplastic Infantile Astrocytoma; Fibrous Dysplasia; Paratyphoid Fever; Lesch-Nyhan Syndrome; Sudden Infant Death Syndrome; HIV Infections; Rasmussen Encephalitis; Musculoskeletal Diseases; Wernicke-Korsakoff Syndrome; Acute Fatty Liver of Pregnancy; Binge Drinking; Compassion Fatigue; Enterocolitis, Neutropenic; Non-ST Elevated Myocardial Infarction; Infarction, Posterior Cerebral Artery; Short Bowel Syndrome; Granulomatous Slack Skin Disease; Porphyrias, Hepatic; Maternal Death; Tropical Sprue; Ankylosis of Teeth; Causalgia; Desmoplastic Small Round Cell Tumor; Fibrous Dysplasia of Bone; Parental Death; Lethargy; Sudden Sensorineural Hearing Loss; HIV Seropositivity; Raynaud Disease; Musculoskeletal Pain; West Nile Encephalitis; Acute Febrile Encephalopathy; Binge-Eating Disorder; Complement Component 5 Deficiency; Enterocolitis, Pseudomembranous; Nonbullous Congenital Ichthyosiform Erythroderma; Infection; Shoulder Dislocation; Granulosa Cell Tumor; Port-Wine Stain; Maternally Inherited Diabetes and Deafness; Truncus Arteriosus, Persistent; Anodontia; CD4 Deficiency; Developmental Disabilities; Fibrous Dysplasia, Polyostotic; Paresis; Leucocyte Adhesion Defect; Suicidal Ideation; HIV Wasting Syndrome; Reactive Arthritis; Mushroom Poisoning; West Nile Fever; Acute Flaccid Myelitis; Biotinidase Deficiency; Complement Component Deficiency; Enteropathica; Noncommunicable Diseases; Infectious Arthritis; Shoulder Fractures; Granulosa Cell Tumor of the Ovary; Positive-Pressure Respiration, Intrinsic; Maturity-onset Diabetes of the Young; Trypanosomiasis; Anomia; CDKL5 Deficiency Disorder; Developmental Dysphasia Familial; Filariasis; Paresthesia; Leukemia; Suicide; HIV-Associated Lipodystrophy Syndrome; Reactive Attachment Disorder; Mutism; West Nile Virus; Acute Generalized Exanthematous Pustulosis; Bipolar and Related Disorders; Complete Atrioventricular Canal; Enteropathy-Associated T-Cell Lymphoma; Nondystrophic Myotonia; Infectious Encephalitis; Shoulder Impingement Syndrome; Graves Disease; Posner-Schlossman Syndrome; Maturity-onset Diabetes of the Young, Type 2; Trypanosomiasis, African; Anophthalmos; Cecal Diseases; Developmental Prosopagnosia; Filoviridae Infections; Parkes Weber Syndrome; Leukemia, B-Cell; Suicide, Assisted; Hoarding Disorder; Recessive Dystrophic Epidermolysis Bullosa; Myalgia; West Syndrome; Acute Graft Versus Host Disease; Bipolar Disorder; Complex Regional Pain Syndromes; Enterovirus Infections; Nonseminomatous Germ Cell Tumor; Infectious Mononucleosis; Shoulder Injuries; Graves Ophthalmopathy; Post Polio Syndrome; Maturity-onset Diabetes of the Young, Type 3; Trypanosomiasis, Human East-African; Anorectal Malformations; Celiac Disease; Dextrocardia; Finger Injuries; Parkinson Disease; Leukemia, B-cell, Chronic; Suicide, Attempted; Hoarseness; Rectal Diseases; Myasthenia Gravis; Western Equine Encephalitis; Acute Intermittent Porphyria; Bird Fancier's Lung; Composite Lymphoma; Enthesitis-related Juvenile Idiopathic Arthritis; Nontuberculous Mycobacterial Lung Disease; Infective Endocarditis; Shoulder Pain; Gray Platelet Syndrome; Post-Concussion Syndrome; Maxillary Diseases; Tuberculoma; Anorexia; Cell Transformation, Neoplastic; Di Guglielmo's Syndrome; Firesetting Behavior; Parkinson Disease, Secondary; Leukemia, Basophilic, Acute; Sulfatidosis; Hodgkin Disease; Rectal Fistula; Myasthenia Gravis, Neonatal; Wet Macular Degeneration; Acute Interstitial Pneumonia; Birdshot Chorioretinopathy; Compulsive Behavior; Enthesopathy; Noonan Syndrome; Inferior Wall Myocardial Infarction; Shprintzen-Goldberg Craniosynostosis Syndrome; Gray Zone Lymphoma; Post-Dural Puncture Headache; Maxillary Neoplasms; Tuberculosis; Anorexia Nervosa; Cellulite; Diabetes Complications; Fissure in Ano; Parkinsonian Disorders; Leukemia, Biphenotypic, Acute; Sulfite Oxidase Deficiency; Hodgkin Lymphoma; Rectal Neoplasms; Myasthenic Syndromes, Congenital; Wheat Hypersensitivity; Acute Kidney Injury; Birt-Hogg-Dube Syndrome; Compulsive Personality Disorder; Entropion; Norrie Disease; Infertility; Shwachman-Diamond Syndrome; Griscelli Syndrome; Post-Exercise Hypotension; Maxillary Sinusitis; Tuberculosis, Central Nervous System; Anovulation; Cellulitis; Diabetes Insipidus; Fistula; Paronychia; Leukemia, Eosinophilic, Acute; Sunburn; Holoprosencephaly; Rectal Prolapse; Mycetoma; WHIM Syndrome; Acute Leukemia of Ambiguous Lineage; Birth Injuries; Conduct Disorder; Enuresis; Nose Deformities, Acquired; Infertility, Female; Shy-Drager Syndrome; Growth Disorders; Post-Lyme Disease Syndrome; Maxillofacial Abnormalities; Tuberculosis, Lymph Node; Anterior Compartment Syndrome; Central Centrifugal Cicatricial Alopecia; Diabetes Insipidus, Nephrogenic; Flail Chest; Parotid Diseases; Leukemia, Erythroblastic, Acute; Sunstroke; Homocystinuria; Rectocele; Mycobacterium Abscessus; Whiplash Injuries; Acute Lung Injury; Birth Weight; Condylomata Acuminata; Environmental Illness; Nose Diseases; Infertility, Male; Sialadenitis; Growth Hormone Deficiency; Post-transplant Lymphoproliferative Disease; Maxillofacial Injuries; Tuberculosis, Meningeal; Anterior Cruciate Ligament Injuries; Central Congenital Hypothyroidism; Diabetes Insipidus, Neurogenic; Flank Pain; Parotid Neoplasms; Leukemia, Hairy Cell; Superficial Spreading Melanoma; Homocystinuria Due to CBS Deficiency; Rectovaginal Fistula; Mycobacterium Avium Complex Infections; Whipple Disease; Acute Lymphoblastic Leukemia; Bisphosphonate-Associated Osteonecrosis of the Jaw; Cone Dystrophy; Eosinophilia; Nose Neoplasms; Inflammation; Sialic Acid Storage Disease; Growth Hormone-Secreting Pituitary Adenoma; Post-traumatic Epilepsy; May-Thurner Syndrome; Tuberculosis, Miliary; Anterior Ischemic Optic Neuropathy; Central Cord Syndrome; Diabetes Mellitus; Flatfoot; Parotitis; Leukemia, Large Granular Lymphocytic; Superinfection; Homologous Wasting Disease; Recurrence; Mycobacterium avium-intracellulare Infection; White Coat Hypertension; Acute Megakaryoblastic Leukemia; Bites and Stings; Cone-Rod Dystrophies; Eosinophilia-Myalgia Syndrome; Nuchal Cord; Inflammatory Bowel Diseases; Sialorrhea; GTP Cyclohydrolase I Deficiency; Post-Traumatic Headache; McCune-Albright Syndrome; Tuberculosis, Multidrug-Resistant; Anterior Uveitis; Central Core Disease; Diabetes Mellitus, Experimental; Flatulence; Paroxysmal Hemicrania; Leukemia, Lymphocytic, Chronic, B-Cell; Superior Limbic Keratoconjunctivitis; Hookworm Infections; Recurrent Hydatidiform Mole; Mycobacterium Infections; Whooping Cough; Acute Monoblastic Leukemia; Bites, Human; Cone-rod Dystrophy 2; Eosinophilic Enteropathy; Nut and Peanut Hypersensitivity; Inflammatory Breast Cancer; Sick Sinus Syndrome; Guillain-Barre Syndrome; Postcholecystectomy Syndrome; Measles; Tuberculosis, Osteoarticular; Anterior Wall Myocardial Infarction; Central Diabetes Insipidus; Diabetes Mellitus, Lipoatrophic; Flaviviridae Infections; Paroxysmal Kinesigenic Choreoathetosis; Leukemia, Lymphoid; Superior Mesenteric Artery Syndrome; Hordeolum; Recurrent Laryngeal Nerve Injuries; Mycobacterium Infections, Nontuberculous; Wiedemann-Steiner Syndrome; Acute Mountain Sickness; BK-virus Nephropathy; Confusion; Eosinophilic Esophagitis; Nut Hypersensitivity; Inflammatory Breast Neoplasms; Sickle Beta Thalassemia; Gynandroblastoma; Posterior Column Ataxia; Meckel Syndrome; Tuberculosis, Pleural; Anthrax; Central Nervous System Bacterial Infections; Diabetes Mellitus, Type 1; Flavivirus Infections; Paroxysmal Nocturnal Hemoglobinuria; Leukemia, Mast-Cell; Superior Vena Cava Syndrome; Horizontal Gaze Palsy With Progressive Scoliosis; Recurrent Respiratory Papillomatosis; Mycobacterium Xenopi; Williams Syndrome; Acute Myeloblastic Leukemia With Maturation; Bladder Exstrophy; Congenital Abnormalities; Eosinophilic Fasciitis; Nutrition Disorders; Inflammatory Myofibroblastic Tumor; Sickle Cell Anemia; Gynatresia; Posterior Leukoencephalopathy Syndrome; Meconium Aspiration Syndrome; Tuberculosis, Pulmonary; Anti-Glomerular Basement Membrane Disease; Central Nervous System Cysts; Diabetes Mellitus, Type 2; Fluoride Poisoning; Paroxysmal Ventricular Fibrillation; Leukemia, Megakaryoblastic, Acute; Suppuration; Horner Syndrome; Red-Cell Aplasia, Pure; Mycoplasma Infections; Wilms Tumor; Acute Myeloblastic Leukemia Without Maturation; Blast Crisis; Congenital Adrenal Hyperplasia; Eosinophilic Granulomatosis With Polyangiitis; Nystagmus, Congenital; Influenza in Birds; Sickle Cell Trait; Gynecomastia; Posterior Tibial Tendon Dysfunction; Meconium Ileus; Tuberculosis, Spinal; Anti-N-Methyl-D-Aspartate Receptor Encephalitis; Central Nervous System Diseases; Diabetes, Gestational; Fluorosis, Dental; Pars Planitis; Leukemia, Monocytic, Acute; Supraglottic Laryngeal Cancer; Hot Flashes; Reducing Body Myopathy; Mycoplasmal Pneumonia; Wilson Disease; Acute Myeloid Leukemia; Blast Injuries; Congenital Amegakaryocytic Thrombocytopenia; Ependymoma; Nystagmus, Pathologic; Influenza, Human; Sideroblastic Anemia Pyridoxine-refractory Autosomal Recessive; Gyrate Atrophy; Posterior Urethral Valves; MECP2 Duplication Syndrome; Tuberculous Meningitis; Anti-Neutrophil Cytoplasmic Antibody-Associated Vasculitis; Central Nervous System Fungal Infections; Diabetic Angiopathies; Flushing; Parsonage Turner Syndrome; Leukemia, Myelogenous, Chronic, BCR-ABL Positive; Supranuclear Ocular Palsy; Hoyeraal Hreidarsson Syndrome; Refeeding Syndrome; Mycoses; Wiskott-Aldrich Syndrome; Acute Myelomonocytic Leukemia; Blastic Plasmacytoid Dendritic Cell; Congenital Aplastic Anemia; Epidermal Cyst; Obesity; Infratentorial Neoplasms; Siderosis; Gyrate Atrophy of Choroid and Retina; Posterior Uveitis; Medial Tibial Stress Syndrome; Tuberculous Uveitis; Anticholinergic Syndrome; Central Nervous System Germinoma; Diabetic Cardiomyopathies; FMR1-related Primary Ovarian Insufficiency; Partial Deletion of Y; Leukemia, Myeloid; Supranuclear Palsy, Progressive; HTLV-1 Associated Myelopathy/tropical Spastic Paraparesis; Reflex Sympathetic Dystrophy; Mycosis Fungoides; Wolf-Hirschhorn Syndrome; Acute Non Lymphoblastic Leukemia; Blastocystis Infections; Congenital Central Hypoventilation Syndrome; Epidermodysplasia Verruciformis; Obesity Hypoventilation Syndrome; Inhalant Abuse; Sigmoid Diseases; Haemophilus Infections; Postoperative Complications; Median Neuropathy; Tuberous Sclerosis; Anticipation, Genetic; Central Nervous System Infections; Diabetic Foot; Focal Cortical Dysplasia of Taylor; Parvoviridae Infections; Leukemia, Myeloid, Accelerated Phase; Supratentorial Neoplasms; HTLV-I Infections; Reflex, Abnormal; Mydriasis; Wolff-Parkinson-White Syndrome; Acute Pain; Blastomycosis; Congenital Contractural Arachnodactyly; Epidermolysis Bullosa; Obesity, Abdominal; Injection Site Reaction; Sigmoid Neoplasms; Haemophilus Influenzae; Postoperative Hemorrhage; Mediastinal Diseases; Tubular Aggregate Myopathy; Antiphospholipid Syndrome; Central Nervous System Neoplasms; Diabetic Ketoacidosis; Focal Dermal Hypoplasia; PASLI Disease; Leukemia, Myeloid, Acute; Supratentorial Primitive Neuroectodermal Tumor; HTLV-II Infections; Refractive Errors; Myelitis; Wolfram Syndrome; Acute Promyelocytic Leukemia; Blepharitis; Congenital Cytomegalovirus; Epidermolysis Bullosa Acquisita; Obesity, Metabolically Benign; Insect Bites and Stings; Signs and Symptoms, Digestive; Hailey-Hailey Disease; Postoperative Nausea and Vomiting; Mediastinal Emphysema; Tufted Angioma; Antisocial Personality Disorder; Central Nervous System Vascular Malformations; Diabetic Nephropathies; Focal Infection; Passive-Aggressive Personality Disorder; Leukemia, Myeloid, Chronic, Atypical, BCR-ABL Negative; Supravalvular Aortic Stenosis; Human T-cell Leukemia Virus Type 1; Refsum Disease; Myelitis, Transverse; Wolman Disease; Acute Radiation Syndrome; Blepharoptosis; Congenital Diaphragmatic Hernia; Epidermolysis Bullosa Dystrophica; Obesity, Morbid; Insomnia, Fatal Familial; Signs and Symptoms, Respiratory; Hair Diseases; Postpartum Hemorrhage; Mediastinal Neoplasms; Tufting Enteropathy; Antisynthetase Syndrome; Central Nervous System Viral Diseases; Diabetic Neuropathies; Focal Infection, Dental; Patellar Dislocation; Leukemia, Myeloid, Chronic-Phase; Surgical Wound; Human T-cell Leukemia Virus Type 2; Refsum Disease, Infantile; Myelodysplastic Syndrome With Excess Blasts; Wound Infection; Acute Respiratory Distress Syndrome; Blepharospasm; Congenital Disorders of Glycosylation; Epidermolysis Bullosa Simplex; Obsessive Behavior; Insulin Resistance; Silicosis; Hairy Cell Leukemia; Postpartum Thyroiditis; Mediastinitis; Tularemia; Antithrombin III Deficiency; Central Neurocytoma; Diabetic Retinopathy; Focal Nodular Hyperplasia; Patellofemoral Pain Syndrome; Leukemia, Myelomonocytic, Acute; Surgical Wound Dehiscence; Humeral Fractures; Refsum Disease, Infantile Form; Myelodysplastic Syndromes; Wounds and Injuries; Acute Zonal Occult Outer Retinopathy; Blind Loop Syndrome; Congenital Dyserythropoietic Anemia; Epidermolysis Bullosa Simplex, Generalized; Obsessive-Compulsive Disorder; Insulin-like Growth Factor I Deficiency; Silver-Russell Syndrome; Halitosis; Postpericardiotomy Syndrome; Medically Unexplained Symptoms; Tumefactive Multiple Sclerosis; Anuria; Central Pain Syndrome; Diamond-Blackfan Anemia; Focal Segmental Glomerulosclerosis; Patent Ductus Arteriosus; Leukemia, Myelomonocytic, Chronic; Surgical Wound Infection; Huntington Disease; Relapsing Fever; Myelodysplastic-Myeloproliferative Diseases; Wounds, Gunshot; Acute-On-Chronic Liver Failure; Blindness; Congenital Dyserythropoietic Anemia Type 1; Epidermolysis Bullosa Simplex, Localized; Obstetric Labor Complications; Insulinoma; Single Ventricular Heart; Hallucinations; Postphlebitic Syndrome; Medium-chain Acyl-coenzyme A Dehydrogenase Deficiency; Tumor Lysis Syndrome; Anus Diseases; Central Serous Chorioretinopathy; Diaper Rash; Folic Acid Deficiency; Pathological Conditions, Anatomical; Leukemia, Myelomonocytic, Juvenile; Susac Syndrome; Hurler Syndrome; Relapsing Polychondritis; Myelodysplastic/myeloproliferative Disease; Wounds, Nonpenetrating; Acute-Phase Reaction; Blister; Congenital Erythropoietic Porphyria; Epidermolysis Bullosa, Junctional; Obstetric Labor, Premature; Intellectual Disability; Sinonasal Undifferentiated Carcinoma; Hallux Limitus; Postpoliomyelitis Syndrome; Medulloblastoma; Tumor Necrosis Factor Receptor-associated Periodic Syndrome; Anus Neoplasms; Centronuclear Myopathy; Diaphragmatic Eventration; Follicular Cyst; Pauciarticular Onset Juvenile Idiopathic Arthritis; Leukemia, Neutrophilic, Chronic; Swayback; Hurlerâ€″Scheie Syndrome; REM Sleep Behavior Disorder; Myelofibrosis; Wounds, Penetrating; Adamantinoma; Blood Coagulation Disorders; Congenital Fiber Type Disproportion; Epidermolytic Ichthyosis; Occipital Horn Syndrome; Intermittent Claudication; Sinus Cancer; Hallux Rigidus; Postthrombotic Syndrome; Medulloblastoma, Childhood; Tumor Virus Infections; Anxiety Disorders; Cerebellar Astrocytoma, Childhood; Diaphragmatic Hernia Exomphalos Corpus Callosum Agenesis; Follicular Lymphoma; Peanut Hypersensitivity; Leukemia, Plasma Cell; Sweat Gland Diseases; Hurthle Cell Thyroid Cancer; REM Sleep Parasomnias; Myeloid Leukemia; Wrist Injuries; Adams-Stokes Syndrome; Blood Coagulation Disorders, Inherited; Congenital Fibrosis of Extraocular Muscles; Epididymitis; Occupational Diseases; Intertrigo; Sinus Thrombosis, Intracranial; Hallux Valgus; Postural Orthostatic Tachycardia Syndrome; Megacolon; Turcot Syndrome; Anxiety, Separation; Cerebellar Ataxia; Diarrhea; Folliculitis; Pearson Syndrome; Leukemia, Prolymphocytic; Sweat Gland Neoplasms; Hutchinson's Melanotic Freckle; Remission, Spontaneous; Myeloid Sarcoma; X-linked Adrenal Hypoplasia Congenita; Addison Disease; Blood Loss, Surgical; Congenital Generalized Fibromatosis; Epidural Neoplasms; Occupational Injuries; Intervertebral Disc Degeneration; Sinusitis; Hallux Varus; Potassium Aggravated Myotonia; Megalencephaly; Turner Syndrome; Aortic Aneurysm; Cerebellar Ataxia and Hypogonadotropic Hypogonadism; Diarrhea, Infantile; Food Addiction; Pectus Carinatum; Leukemia, Prolymphocytic, B-Cell; Sweating, Gustatory; Hyaline Fibromatosis Syndrome; Renal Agenesis; Myelomeningocele; X-linked Adrenoleukodystrophy; Adenine Phosphoribosyltransferase Deficiency; Blood Platelet Disorders; Congenital Generalized Lipodystrophy; Epiglottitis; Occupational Stress; Intervertebral Disc Displacement; Sitosterolemia; Hamartoma; Potassium Deficiency; Melanoma; Twin to Twin Transfusion Syndrome; Aortic Aneurysm, Abdominal; Cerebellar Ataxia Ectodermal Dysplasia; Diastasis, Bone; Food Hypersensitivity; Pediatric Acute-onset Neuropsychiatric Syndrome; Leukemia, Prolymphocytic, T-Cell; Sweet Syndrome; Hyaline Membrane Disease; Renal Artery Obstruction; Myeloproliferative Disorders; X-linked Agammaglobulinemia; Adenocarcinoma; Blood Protein Disorders; Congenital Heart Block; Epilepsia Partialis Continua; Ochronosis; Intestinal Atresia; Situs Inversus; Hamartoma Syndrome, Multiple; Potocki-Shaffer Syndrome; Melanoma, Experimental; Twins, Conjoined; Aortic Aneurysm, Thoracic; Cerebellar Degeneration; Diastasis, Muscle; Food Intolerance; Pediatric Crohn's Disease; Leukemia, Promyelocytic, Acute; Sydenham's Chorea; Hyalinosis, Systemic; Renal Cell Carcinoma; MYH-associated Polyposis; X-Linked Combined Immunodeficiency Diseases; Adenocarcinoma in Situ; Bloom Syndrome; Congenital Hemolytic Anemia; Epilepsies, Myoclonic; Ocular Cicatricial Pemphigoid; Intestinal Diseases; Sixth Nerve Palsy; Hammer Toe Syndrome; Pouchitis; Melanoma, Familial; Tympanic Membrane Perforation; Aortic Arch Syndromes; Cerebellar Diseases; Diastema; Foot Deformities; Pediatric Hypertension; Leukemia, T-Cell; Symptom Flare Up; Hydatidiform Mole; Renal Cell Carcinoma 4; MYH9 Related Thrombocytopenia; X-linked Congenital Stationary Night Blindness; Adenocarcinoma of Lung; Blue Rubber Bleb Nevus Syndrome; Congenital Hepatic Fibrosis; Epilepsies, Partial; Ocular Hypertension; Intestinal Diseases, Parasitic; Sjogren's Syndrome; Hand Deformities; Poxviridae Infections; Melanosis; Type 1 Plasminogen Deficiency; Aortic Coarctation; Cerebellar Hypoplasia; DICER1-related Pleuropulmonary Blastoma Cancer Predisposition Syndrome; Foot Deformities, Acquired; Pediatric Multiple Sclerosis; Leukemia, T-cell, Chronic; Syncope; Hydatidiform Mole, Invasive; Renal Colic; Myocardial Bridging; X-linked Creatine Deficiency; Adenocarcinoma of the Appendix; Body Dysmorphic Disorders; Congenital Herpes Simplex; Epilepsy; Ocular Hypotension; Intestinal Fistula; Sjogren-Larsson Syndrome; Hand Deformities, Congenital; Prader-Willi Syndrome; MELAS Syndrome; Typhlitis; Aortic Diseases; Cerebellar Liponeurocytoma; Diencephalic Syndrome; Foot Deformities, Congenital; Pediatric Obesity; Leukemia-Lymphoma, Adult T-Cell; Syncope, Vasovagal; Hydatidosis; Renal Insufficiency; Myocardial Infarction; X-linked Hypohidrotic Ectodermal Dysplasia; Adenocarcinoma, Bronchiolo-Alveolar; Body Temperature Changes; Congenital Human Immunodeficiency Virus; Epilepsy Juvenile Absence; Ocular Melanoma; Intestinal Lymphangiectasia; Skin Abnormalities; Hand Dermatoses; Pre-Eclampsia; Melena; Typhoid Fever; Aortic Rupture; Cerebellar Neoplasms; Dientamoebiasis; Foot Dermatoses; Pediatric Ulcerative Colitis; Leukemoid Reaction; Syndactyly; Hydranencephaly; Renal Insufficiency, Chronic; Myocardial Ischemia; X-linked Hypophosphatemia; Adenocarcinoma, Clear Cell; Body Weight; Congenital Hydrocephalus; Epilepsy With Myoclonic-atonic Seizures; Ocular Motility Disorders; Intestinal Neoplasms; Skin Diseases; Hand Injuries; Pre-Excitation Syndromes; Melioidosis; Typhus; Aortic Stenosis, Supravalvular; Cerebral Amyloid Angiopathy; Diffuse Astrocytoma; Foot Diseases; Pedophilia; Leukoaraiosis; Syndrome; Hydrarthrosis; Renal Medullary Carcinoma; Myocardial Reperfusion Injury; X-linked Lymphoproliferative Syndrome; Adenocarcinoma, Follicular; Body Weight Changes; Congenital Hyperinsulinism; Epilepsy, Absence; Ocular Muscular Dystrophy; Intestinal Obstruction; Skin Diseases, Bacterial; Hand, Foot and Mouth Disease; Precancerous Conditions; Melorheostosis; Typhus, Epidemic Louse-Borne; Aortic Valve Insufficiency; Cerebral Amyloid Angiopathy, Familial; Diffuse Axonal Injury; Foot Injuries; Pelizaeus-Merzbacher Disease; Leukocyte Adhesion Deficiency Type 1; Synkinesis; Hydrocephalus; Renal Oncocytoma; Myocardial Stunning; X-linked Lymphoproliferative Syndrome 2; Adenocarcinoma, Mucinous; Body-Weight Trajectory; Congenital Hypothyroidism; Epilepsy, Complex Partial; Ocular Toxoplasmosis; Intestinal Perforation; Skin Diseases, Eczematous; Hand-Arm Vibration Syndrome; Precocious Puberty; Membranoproliferative Glomerulonephritis; Tyrosinemia Type 1; Aortic Valve Stenosis; Cerebral Arterial Diseases; Diffuse Cerebral Sclerosis of Schilder; Foot Ulcer; Pellucid Marginal Degeneration; Leukocyte Disorders; Synovial Cyst; Hydrocephalus, Normal Pressure; Renal Tubular Acidosis; Myocarditis; X-linked Myotubular Myopathy; Adenocarcinoma, Papillary; Bohring-Opitz Syndrome; Congenital Insensitivity to Pain; Epilepsy, Frontal Lobe; Oculocerebrorenal Syndrome; Intestinal Polyposis; Skin Diseases, Genetic; Hand-Foot Syndrome; Precocious Puberty, Gonadotropin-dependent; Membranous Nephropathy; Tyrosinemia Type 2; Aortitis; Cerebral Astrocytoma, Childhood; Diffuse Cutaneous Systemic Sclerosis; Foot-and-Mouth Disease; Pelvic Floor Disorders; Leukocyte-Adhesion Deficiency Syndrome; Synovial Sarcoma; Hydronephrosis; Renal Tubular Acidosis, Distal, Autosomal Dominant; Myoclonic Epilepsies, Progressive; X-linked Severe Combined Immunodeficiency; Adenocarcinoma, Scirrhous; Bone Cysts; Congenital Lymphedema; Epilepsy, Generalized; Oculocutaneous Albinism; Intestinal Polyps; Skin Diseases, Infectious; Hansen's Disease; Precursor B-Cell Lymphoblastic Leukemia-Lymphoma; Memory Disorders; Tyrosinemia Type 3; Aphakia; Cerebral Hemorrhage; Diffuse Gastric Cancer; Foramen Ovale, Patent; Pelvic Girdle Pain; Leukocytosis; Synovitis; Hydrophthalmos; Renal Tubular Transport, Inborn Errors; Myoclonic Epilepsy, Juvenile; X-linked Thrombocytopenia; Adenocarcinoma, Sebaceous; Bone Cysts, Aneurysmal; Congenital Mesoblastic Nephroma; Epilepsy, Partial, Motor; Oculomotor Nerve Diseases; Intestinal Pseudo-Obstruction; Skin Diseases, Metabolic; Hantavirus Infections; Precursor Cell Lymphoblastic Leukemia-Lymphoma; Mendelian Susceptibility to Mycobacterial Diseases; Tyrosinemias; Aphakia, Postcataract; Cerebral Hemorrhage, Traumatic; Diffuse Intrinsic Pontine Glioma; Forearm Injuries; Pelvic Infection; Leukodystrophy; Synovitis, Pigmented Villonodular; Hydrops Fetalis; Reperfusion Injury; Myoclonus; Xanthogranuloma, Juvenile; Adenofibroma; Bone Demineralization, Pathologic; Congenital Microtia; Epilepsy, Post-Traumatic; Oculopharyngeal Muscular Dystrophy; Intestinal Volvulus; Skin Diseases, Papulosquamous; Hantavirus Pulmonary Syndrome; Precursor T-Cell Lymphoblastic Leukemia-Lymphoma; Menetrier Disease; Ulcer; Aphasia; Cerebral Infarction; Diffuse Large B-Cell Lymphoma; Foreign Bodies; Pelvic Inflammatory Disease; Leukodystrophy, Globoid Cell; Syphilis; Hydrothorax; Reproductive Tract Infections; Myoclonus Epilepsy; Xanthoma Disseminatum; Adenoid Cystic Carcinoma; Bone Diseases; Congenital Mirror Movement Disorder; Epilepsy, Reflex; Odontogenic Tumors; Intra-Abdominal Hypertension; Skin Diseases, Parasitic; Hashimoto Disease; Prediabetic State; Meniere Disease; Ulcerative Proctitis; Aphasia, Broca; Cerebral Palsy; DiGeorge Syndrome; Foreign-Body Reaction; Pelvic Neoplasms; Leukodystrophy, Metachromatic; Syphilis, Congenital; Hyper IgE Syndrome; Respiration Disorders; Myoclonus-dystonia; Xanthomatosis; Adenoma; Bone Diseases, Developmental; Congenital Muscular Dystrophy; Epilepsy, Rolandic; Ogilvie Syndrome; Intra-Articular Fractures; Skin Diseases, Vesiculobullous; Hashimoto Encephalopathy; Pregnancy Complications; Meningeal Carcinomatosis; Ullrich Congenital Muscular Dystrophy; Aphasia, Conduction; Cerebral Palsy Ataxic; Digestive System Abnormalities; Fournier Gangrene; Pelvic Organ Prolapse; Leukoencephalopathies; Syphilis, Latent; Hyper-IgD Syndrome; Respiratory Aspiration; Myoepithelial Carcinoma; Xanthomatosis, Cerebrotendinous; Adenoma of the Adrenal Gland; Bone Diseases, Endocrine; Congenital Muscular Dystrophy Due to Dystroglycanopathy; Epilepsy, Temporal Lobe; Olfaction Disorders; Intraabdominal Infections; Skin Diseases, Viral; Head and Neck Neoplasms; Pregnancy Complications, Cardiovascular; Meningeal Neoplasms; Ulna Fractures; Aphasia, Primary Progressive; Cerebral Palsy Athetoid; Digestive System Diseases; Fowler's Syndrome; Pelvic Pain; Leukoencephalopathy With Vanishing White Matter; Syringoma; Hyper-IgM Immunodeficiency Syndrome; Respiratory Aspiration of Gastric Contents; Myofascial Pain Syndromes; Xeroderma Pigmentosum; Adenoma, Acidophil; Bone Diseases, Infectious; Congenital Muscular Dystrophy Type 1A; Epilepsy, Tonic-Clonic; Olfactory Nerve Injuries; Intracranial Aneurysm; Skin Manifestations; Head Injuries, Closed; Pregnancy Complications, Hematologic; Meningioma; Ulnar Nerve Compression Syndromes; Aphasia, Wernicke; Cerebral Palsy Spastic Hemiplegic; Digestive System Fistula; Fowlpox; Pemphigoid, Benign Mucous Membrane; Leukoencephalopathy, Progressive Multifocal; Syringomyelia; Hyper-IgM Immunodeficiency Syndrome, Type 1; Respiratory Distress Syndrome, Adult; Myofibrillar Myopathy; Xerophthalmia; Adenoma, Basophil; Bone Diseases, Metabolic; Congenital Myasthenic Syndrome; Epileptic Syndromes; Olfactory Neuroblastoma; Intracranial Arterial Diseases; Skin Neoplasms; Head Injuries, Penetrating; Pregnancy Complications, Infectious; Meningitis; Ulnar Neuropathies; Aphonia; Cerebral Small Vessel Diseases; Digestive System Neoplasms; FOXG1 Syndrome; Pemphigoid, Bullous; Leukomalacia, Periventricular; Systemic Candidiasis; Hyperacusis; Respiratory Distress Syndrome, Infant; Myofibroma; Xerostomia; Adenoma, Bile Duct; Bone Malalignment; Congenital Myotonic Dystrophy; Epiphyseal Dysplasia Multiple With Early-onset Diabetes Mellitus; Oligoastrocytoma; Intracranial Arteriosclerosis; Skin Ulcer; Headache; Pregnancy Complications, Neoplastic; Meningitis, Aseptic; Unconsciousness; Aphthous Stomatitis; Cerebrospinal Fluid Leak; Dihydroxyadeninuria; Fracture Dislocation; Pemphigus; Leukopenia; Systemic Capillary Leak Syndrome; Hyperadrenalism; Respiratory Distress Syndrome, Newborn; Myofibromatosis; XYY Karyotype; Adenoma, Chromophobe; Bone Marrow Diseases; Congenital Pseudoarthrosis; Epiretinal Membrane; Oligodendroglioma; Intracranial Arteriovenous Malformations; Skull Base Neoplasms; Headache Disorders; Pregnancy in Diabetics; Meningitis, Bacterial; Underage Drinking; Aplasia Cutis Congenita; Cerebrotendinous Xanthomatosis; Dilatation, Pathologic; Fractures, Avulsion; Pemphigus Foliaceus; Leukoplakia; Systemic Inflammatory Response Syndrome; Hyperaldosteronism; Respiratory Hypersensitivity; Myoglobinuria; Y Chromosome Infertility; Adenoma, Islet Cell; Bone Marrow Neoplasms; Congenital Rubella; Episodic Ataxia; Oligohydramnios; Intracranial Embolism; Skull Fractures; Headache Disorders, Primary; Pregnancy, Abdominal; Meningitis, Cryptococcal; Undifferentiated Connective Tissue Diseases; Aplastic Anemia; Cerebrovascular Disorders; Dilated Cardiomyopathy; Fractures, Bone; Pemphigus Vulgaris; Leukoplakia, Hairy; Systemic Mastocytosis; Hyperalgesia; Respiratory Insufficiency; Myokymia; Yang Deficiency; Adenoma, Liver Cell; Bone Neoplasms; Congenital Sucrase-isomaltase Deficiency; Episodic Ataxia With Nystagmus; Oligomenorrhea; Intracranial Embolism and Thrombosis; Sleep Apnea Syndromes; Headache Disorders, Secondary; Pregnancy, Cornual; Meningitis, Fungal; Undifferentiated Pleomorphic Sarcoma; Apnea; Cerebrovascular Trauma; Dilated Cardiomyopathy With Hypergonadotropic Hypogonadism; Fractures, Closed; Pemphigus, Benign Familial; Leukoplakia, Oral; Systemic Onset Juvenile Idiopathic Arthritis; Hyperammonemia; Respiratory Paralysis; Myoma; Yaws; Adenoma, Pleomorphic; Bone Resorption; Congenital Sucrose Isomaltose Malabsorption; Epispadias; Oligospermia; Intracranial Hemorrhage, Hypertensive; Sleep Apnea, Central; Healthcare-Associated Pneumonia; Pregnancy, Ectopic; Meningitis, Meningococcal; Unicentric Castleman Disease; Apocrine Carcinoma; Ceroid Storage Disease; Dipetalonema Infections; Fractures, Comminuted; Penile Cancer, Adult; Levator Syndrome; Systemic Scleroderma; Hyperandrogenism; Respiratory Sounds; Myopathies, Nemaline; Yellow Fever; Adenoma, Villous; Borderline Personality Disorder; Congenital Thrombotic Thrombocytopenia Purpura; Epistaxis; Oliguria; Intracranial Hemorrhage, Traumatic; Sleep Apnea, Obstructive; Hearing Disorders; Pregnancy, Interstitial; Meningitis, Pneumococcal; Unilateral Breast Neoplasms; Apparent Mineralocorticoid Excess; Cervical Dystonia; Diphtheria; Fractures, Compression; Penile Diseases; Lewy Body Disease; Systemic Vasculitis; Hyperargininemia; Respiratory Syncytial Virus Infections; Myopathies, Structural, Congenital; Yersinia Infections; Adenomatous Polyposis Coli; Bordetella Infections; Congenital Torticollis; Epithelial-myoepithelial Carcinoma; Olivopontocerebellar Atrophies; Intracranial Hemorrhages; Sleep Arousal Disorders; Hearing Loss; Pregnancy, Ovarian; Meningitis, Viral; Uniparental Disomy; Appendiceal Neoplasms; Cervical Intraepithelial Neoplasia; Diplopia; Fractures, Malunited; Penile Induration; Leydig Cell Tumor; Systolic Murmurs; Hyperbilirubinemia; Respiratory System Abnormalities; Myopathy Congenital; Yersinia pseudotuberculosis Infections; Adenomatous Polyps; Borrelia Infections; Congenital Tracheal Stenosis; Epithelioid Sarcoma; Omenn Syndrome; Intracranial Hypertension; Sleep Bruxism; Hearing Loss, Bilateral; Pregnancy, Prolonged; Meningocele; Uniparental Disomy of Chromosome 11; Appendicitis; Chagas Cardiomyopathy; Dipsogenic Diabetes Insipidus; Fractures, Multiple; Penile Neoplasms; Li-Fraumeni Syndrome; T Cell Immunodeficiency Primary; Hyperbilirubinemia, Neonatal; Respiratory Tract Diseases; Myopathy, Central Core; Yin Deficiency; Adenomyoepithelioma; Botulism; Congenital Vertical Talus; Epstein-Barr Virus Infections; Onchocerciasis; Intracranial Hypotension; Sleep Deprivation; Hearing Loss, Central; Pregnancy, Tubal; Meningococcal Infections; Unverricht-Lundborg Disease; Apraxia, Ideomotor; Chagas Disease; Discitis; Fractures, Open; Penis Agenesis; Lice Infestations; T-cell Prolymphocytic Leukemia; Hypercalcemia; Respiratory Tract Infections; Myopia; Zellweger Syndrome; Adenomyosis; Bourneville Syndrome; Conjunctival Diseases; Equinus Deformity; Onchocerciasis, Ocular; Intracranial Thrombosis; Sleep Disorders, Circadian Rhythm; Hearing Loss, Conductive; Prehypertension; Meningococcemia; Unverricht-Lundborg Syndrome; Apraxias; Chalazion; Disease Attributes; Fractures, Spontaneous; Pentalogy of Cantrell; Lichen Planopilaris; T-cell/histiocyte Rich Large B Cell Lymphoma; Hypercalciuria; Respiratory Tract Neoplasms; Myopia, Degenerative; Zenker Diverticulum; Adenosarcoma; Bowen's Disease; Conjunctival Neoplasms; Erdheim-Chester Disease; Oncogenic Osteomalacia; Intrahepatic Cholangiocarcinoma; Sleep Disorders, Intrinsic; Hearing Loss, Functional; Preleukemia; Meningoencephalitis; Upper Extremity Deep Vein Thrombosis; Apudoma; Chanarin-Dorfman Syndrome; Disease Progression; Fractures, Stress; Peptic Ulcer; Lichen Planus; T-Lymphocytopenia, Idiopathic CD4-Positive; Hypercapnia; Restless Legs Syndrome; Myosarcoma; Zika Virus Infection; Adenosine Deaminase Deficiency; Brachial Plexus Neuritis; Conjunctivitis; Erectile Dysfunction; Onycholysis; Intrahepatic Cholestasis of Pregnancy; Sleep Initiation and Maintenance Disorders; Hearing Loss, High-Frequency; Premature Birth; Meningomyelocele; Upper Extremity Deformities, Congenital; Aquagenic Pruritus; Chandler's Syndrome; Disease Resistance; Fractures, Ununited; Peptic Ulcer Hemorrhage; Lichen Planus, Oral; Tabes Dorsalis; Hypercholesterolemia; Restless Legs Syndrome, Susceptibility To, 1; Myositis; Zollinger-Ellison Syndrome; Adenosquamous Carcinoma of the Endometrium; Brachial Plexus Neuropathies; Conjunctivitis, Allergic; Erosive Pustular Dermatosis of the Scalp; Onychomycosis; Intraocular Lymphoma; Sleep Wake Disorders; Hearing Loss, Mixed Conductive-Sensorineural; Premature Ejaculation; Menkes Disease; Urachal Cancer; Arachnodactyly; Channelopathies; Disease Susceptibility; Fragile X Syndrome; Peptic Ulcer Perforation; Lichen Sclerosus; Tachycardia; Hyperekplexia; Restless Legs Syndrome, Susceptibility To, 6; Myositis Ossificans; Zoonoses

Adenoviridae Infections; Brachydactyly; Conjunctivitis, Bacterial; Eructation; Open Bite; Intraocular Melanoma; Sleep-Wake Transition Disorders; Hearing Loss, Noise-Induced; Premenstrual Dysphoric Disorder; Menkes Kinky Hair Syndrome; Urachal Cyst; Arachnoiditis; Chaotic Atrial Tachycardia; Diseases in Twins; Frailty; Perceptual Disorders; Lichen Sclerosus et Atrophicus; Tachycardia, Atrioventricular Nodal Reentry; Hyperemesis Gravidarum; Reticular Dysgenesis; Myositis, Inclusion Body; Zygomatic Fractures; Adenovirus Infections, Human; Bradycardia; Conjunctivitis, Inclusion; Erysipelas; Ophthalmia Neonatorum; Intraoperative Awareness; Sleepiness; Hearing Loss, Sensorineural; Premenstrual Syndrome; Menopause; Urea Cycle Disorders; Arbovirus Infections; Charcot-Marie-Tooth Disease; Disorders of Environmental Origin; Fraser Syndrome; Peri-Implantitis; Liddle Syndrome; Tachycardia, Ectopic Atrial; Hyperemia; Reticulohistiocytoma; Myotonia; Zygomycosis; Adenylosuccinase Deficiency; Brain Abscess; Conjunctivitis, Viral; Erythema; Ophthalmoplegia; Intraoperative Complications; Slipped Capital Femoral Epiphyses; Hearing Loss, Sudden; Prenatal Exposure Delayed Effects; Menopause, Premature; Urea Cycle Disorders, Inborn; Arcus Senilis; Charcot-Marie-Tooth Disease Type 1; Disorders of Excessive Somnolence; Frasier Syndrome; Periapical Abscess; Light Chain Deposition Disease; Tachycardia, Ectopic Junctional; Hypereosinophilic Syndrome; Retinal Artery Occlusion; Myotonia Atrophica; Adiposis Dolorosa; Brain Concussion; Connective Tissue Diseases; Erythema Chronicum Migrans; Ophthalmoplegia, Chronic Progressive External; Intravenous Leiomyomatosis; Slow Virus Diseases; Hearing Loss, Unilateral; Presbycusis; Menorrhagia; Ureaplasma Infections; Arginase Deficiency; Charcot-Marie-Tooth Disease Type 1A; Disorders of Sex Development; Freeman Sheldon Syndrome; Periapical Diseases; Lightning Injuries; Tachycardia, Paroxysmal; Hyperesthesia; Retinal Degeneration; Myotonia Congenita; Adjustment Disorders; Brain Contusion; Conotruncal Heart Malformations; Erythema Elevatum Diutinum; Opioid-Related Disorders; Intrinsic Factor Deficiency; Small Cell Lung Cancer; Heart Arrest; Presbyopia; Menstruation Disturbances; Uremia; Argininosuccinic Aciduria; Charcot-Marie-Tooth Disease Type 1B; Disruptive, Impulse Control, and Conduct Disorders; Frey's Syndrome; Periapical Granuloma; Ligneous Conjunctivitis; Tachycardia, Reciprocating; Hypergammaglobulinemia; Retinal Detachment; Myotonic Disorders; Adnexal Diseases; Brain Damage, Chronic; Consciousness Disorders; Erythema Infectiosum; Opportunistic Infections; Intussusception; Small Cell Lung Carcinoma; Heart Block; Pressure Ulcer; Mental Disorders; Ureteral Calculi; Arm Injuries; Charcot-Marie-Tooth Disease Type 2; Disseminated Intravascular Coagulation; Friedreich Ataxia; Periapical Periodontitis; Limb Deformities, Congenital; Tachycardia, Sinus; Hyperglycemia; Retinal Diseases; Myotonic Dystrophy; ADNP Syndrome; Brain Death; Constipation; Erythema Multiforme; Opsoclonus-Myoclonus Syndrome; Invasive Fungal Infections; Small Fiber Neuropathy; Heart Defects, Congenital; Priapism; Mental Fatigue; Ureteral Diseases; Arnold-Chiari Malformation; Charcot-Marie-Tooth Disease Type 2A; Disseminated Peritoneal Leiomyomatosis; Frontal Fibrosing Alopecia; Periarthritis; Limb-girdle Muscular Dystrophy; Tachycardia, Supraventricular; Hyperhidrosis; Retinal Drusen; Myotonic Dystrophy Type 1; Adrenal Cancer; Brain Diseases; Constriction, Pathologic; Erythema Nodosum; Optic Atrophies, Hereditary; Invasive Pulmonary Aspergillosis; Small Intestinal Adenocarcinoma; Heart Diseases; Primary Agammaglobulinemia; Mental Retardation, X-Linked; Ureteral Neoplasms; Aromatic L-amino Acid Decarboxylase Deficiency; Charles Bonnet Syndrome; Dissociative Disorders; Frontal Sinusitis; Pericardial Effusion; Limb-girdle Muscular Dystrophy Type 2A; Tachycardia, Ventricular; Hyperhomocysteinemia; Retinal Dystrophies; Myotonic Dystrophy Type 2; Adrenal Cortex Diseases; Brain Diseases, Metabolic; Continuous Spike-wave During Slow Sleep Syndrome; Erythroblastosis, Fetal; Optic Atrophy; Iridocorneal Endothelial Syndrome; Small Intestine Cancer; Heart Failure; Primary Angiitis of the Central Nervous System; Meralgia Paresthetica; Ureteral Obstruction; Arrhinia; CHD2 Myoclonic Encephalopathy; Dissociative Identity Disorder; Frontotemporal Dementia; Pericarditis; Limb-girdle Muscular Dystrophy Type 2B; Tachypnea; Hyperinsulinism; Retinal Hemorrhage; Myxoid Liposarcoma; Adrenal Cortex Neoplasms; Brain Diseases, Metabolic, Inborn; Contracture; Erythromelalgia; Optic Atrophy 1; Iridocyclitis; Smallpox; Heart Failure, Diastolic; Primary Biliary Cholangitis; Mercury Poisoning; Ureterolithiasis; Arrhythmia, Sinus; Chediak-Higashi Syndrome; Dissociative Seizures; Frontotemporal Dementia, Ubiquitin-positive; Pericarditis, Constrictive; Limb-girdle Muscular Dystrophy Type 2E; Taeniasis; Hyperinsulinism-hyperammonemia Syndrome; Retinal Necrosis Syndrome, Acute; Myxopapillary Ependymoma; Adrenal Gland Diseases; Brain Edema; Contusions; Erythroplakia; Optic Atrophy, Autosomal Dominant; Iris Diseases; Smear Layer; Heart Failure, Systolic; Primary Carnitine Deficiency; Merkel Cell Carcinoma; Urethral Cancer; Arrhythmias, Cardiac; Cheilitis; Distal Arthrogryposis Type 1; Frontotemporal Lobar Degeneration; Pericarditis, Tuberculous; Limb-girdle Muscular Dystrophy Type 21; Takayasu Arteritis; Hyperkalemia; Retinal Neoplasms; N-acetylglutamate Synthase Deficiency; Adrenal Gland Neoplasms; Brain Hemorrhage, Traumatic; Convalescence; Erythroplasia; Optic Atrophy, Hereditary, Leber; Iris Neoplasms; Smith-Lemli-Opitz Syndrome; Heart Injuries; Primary Central Nervous System Lymphoma; Mesangial Proliferative Glomerulonephritis; Urethral Diseases; Arrhythmogenic Right Ventricular Dysplasia; Chemical and Drug Induced Liver Injury; Distal Myopathies; Frostbite; Pericoronitis; Limb-girdle Muscular Dystrophy, Type 2C; Takotsubo Cardiomyopathy; Hyperkalemic Periodic Paralysis; Retinal Neovascularization; Nail Diseases; Adrenal Hyperplasia, Congenital; Brain Infarction; Conversion Disorder; Erythropoietic Protoporphyria; Optic Disk Drusen; Iritis; Smith-Magenis Syndrome; Heart Murmurs; Primary Ciliary Dyskinesia; Mesenchymoma; Urethral Neoplasms; Arsenic Poisoning; Chemical and Drug Induced Liver Injury, Chronic; Distal Renal Tubular Acidosis; Fructose Intolerance; Perilymphatic Fistula; Limb-girdle Muscular Dystrophy, Type 2D; Talipes; Hyperkeratosis, Epidermolytic; Retinal Perforations; Nail-Patella Syndrome; Adrenal Insufficiency; Brain Injuries; Coproporphyria, Hereditary; Escherichia coli Infections; Optic Nerve Diseases; Iron Metabolism Disorders; Smoke Inhalation Injury; Heart Neoplasms; Primary Cutaneous Amyloidosis; Mesenteric Ischemia; Urethral Obstruction; Arterial Calcification and Distal Joint Calcification; Chemotherapy-Induced Febrile Neutropenia; Diurnal Enuresis; Fructose Metabolism, Inborn Errors; Perinatal Death; Limbic Encephalitis; Tangier Disease; Hyperkinesis; Retinal Telangiectasis; Nails, Ingrown; Adrenocortical Adenoma; Brain Injuries, Diffuse; Corneal Diseases; Esophageal Achalasia; Optic Nerve Glioma; Iron Overload; Smoldering Multiple Myeloma; Heart Rupture; Primary Cutaneous Follicle Center Lymphoma; Mesial Movement of Teeth; Urethral Stricture; Arterial Calcification of Infancy; Cherubism; Diverticular Diseases; Fryns Syndrome; Periodontal Atrophy; Limited Cutaneous Systemic Sclerosis; Tardive Dyskinesia; Hyperlactatemia; Retinal Vasculitis; Nails, Malformed; Adrenocortical Carcinoma; Brain Injuries, Traumatic; Corneal Dystrophies, Hereditary; Esophageal and Gastric Varices; Optic Nerve Injuries; Iron-refractory Iron Deficiency Anemia; Snake Bites; Heart Rupture, Post-Infarction; Primary Dysautonomias; Mesothelioma; Urethritis; Arterial Occlusive Diseases; Chest Pain; Diverticulitis; Fuchs' Endothelial Dystrophy; Periodontal Attachment Loss; Limited Systemic Sclerosis; Tarlov Cysts; Hyperlipidemia Type 3; Retinal Vasculopathy With Cerebral Leukodystrophy; Nakajo Nishimura Syndrome; Adrenocortical Hyperfunction; Brain Injury, Chronic; Corneal Edema; Esophageal Atresia; Optic Nerve Neoplasms; Irritable Bowel Syndrome; Sneezing; Heart Septal Defects; Primary Effusion Lymphoma; Metabolic Diseases; Urinary Bladder Calculi; Arterial Tortuosity Syndrome; Cheyne-Stokes Respiration; Diverticulitis, Colonic; Fucosidosis; Periodontal Diseases; Linear IgA Bullous Dermatosis; Taste Disorders; Hyperlipidemia, Familial Combined; Retinal Vein Occlusion; Nakajo Syndrome; Adrenogenital Syndrome; Brain Ischemia; Corneal Endothelial Cell Loss; Esophageal Cancer; Optic Neuritis; Isaacs Syndrome; Snoring; Heart Septal Defects, Atrial; Primary Fanconi Syndrome; Metabolic Side Effects of Drugs and Substances; Urinary Bladder Diseases; Arteriosclerosis; Chiari Malformation Type 2; Diverticulosis, Colonic; Functioning Pancreatic Endocrine Tumor; Periodontal Pocket; Linear IgA Disease; Tauopathies; Hyperlipidemias; Retinitis; Narcolepsy; Adrenoleukodystrophy; Brain Neoplasms; Corneal Endothelial Dystrophy Type 2; Esophageal Diseases; Optic Neuropathy, Ischemic; Ischemia; Social Communication Disorder; Heart Septal Defects, Ventricular; Primary Graft Dysfunction; Metabolic Syndrome; Urinary Bladder Neck Obstruction; Arteriosclerosis Obliterans; Chickenpox; Diverticulum; Fungemia; Periodontitis; Linear Nevus Sebaceous Syndrome; Tay-Sachs Disease; Hyperlipoproteinemia Type 4; Retinitis Pigmentosa; Nasal Obstruction; Adrenomyeloneuropathy; Brain Stem Infarctions; Corneal Injuries; Esophageal Fistula; Optic Pathway Glioma; Ischemic Attack, Transient; Soft Tissue Infections; Heart Tumor; Primary Hyperoxaluria Type 1; Metabolism, Inborn Errors; Urinary Bladder Neoplasms; Arteriovenous Fistula; Chikungunya; Diverticulum, Colon; Funnel Chest; Peripartum Cardiomyopathy; Linear Scleroderma; TDP-43 Proteinopathies; Hyperlipoproteinemia Type 5; Retinitis Pigmentosa 15; Nasal Polyps; Adult Neuronal Ceroid Lipofuscinosis; Brain Stem Neoplasms; Corneal Neovascularization; Esophageal Motility Disorders; Oral Cancer; Isolated ACTH Deficiency; Soft Tissue Injuries; Heart Valve Diseases; Primary Hyperoxaluria Type 2; Metachromatic Leukodystrophy; Urinary Bladder, Neurogenic; Arteriovenous Malformations; Chikungunya Fever; Dizziness; Furcation Defects; Peripheral Arterial Disease; Linitis Plastica; Telangiectasia, Hereditary Hemorrhagic; Hyperlipoproteinemia Type I; Retinitis Pigmentosa-deafness Syndrome; Nasal Septal Perforation; Adult Polyglucosan Body Disease; Brain Tumor, Adult; Corneal Opacity; Esophageal Neoplasms; Oral Hemorrhage; Isolated Growth Hormone Deficiency; Soft Tissue Neoplasms; Heartburn; Primary Hyperparathyroidism; Metal Metabolism, Inborn Errors; Urinary Bladder, Overactive; Arteritis; Chilblains; DNA Repair-Deficiency Disorders; Furunculosis; Peripheral Nerve Injuries; Lip and Oral Cavity Cancer; Telangiectasis; Hyperlipoproteinemia Type II; Retinoblastoma; Nasopharyngeal Carcinoma; ADULT Syndrome; Brain Tumor, Childhood; Corneal Perforation; Esophageal Perforation; Oral Leukoplakia; Isolated Growth Hormone Deficiency Type 1A; Soft Tissue Sarcoma; Heat Exhaustion; Primary Intestinal Lymphangiectasia; Metaplasia; Urinary Bladder, Underactive; Arthralgia; Child Behavior Disorders; DNA Virus Infections; Fusariosis; Peripheral Nervous System Diseases; Lip Neoplasms; Temporomandibular Ankylosis; Hyperlipoproteinemia Type III; Retinopathy of Prematurity; Nasopharyngeal Diseases; Adult T-cell Leukemia/lymphoma; BRCA1 Hereditary Breast and Ovarian Cancer Syndrome; Corneal Ulcer; Esophageal Spasm, Diffuse; Oral Manifestations; Isolated Noncompaction of the Ventricular Myocardium; Solitary Fibrous Tumor, Pleural; Heat Stress Disorders; Primary Lateral Sclerosis; Metaplastic Carcinoma of the Breast; Urinary Calculi; Arthritis; Child Development Disorders, Pervasive; Dominant Optic Atrophy; Gagging; Peripheral Nervous System Neoplasms; Lipedema; Temporomandibular Joint Disorders; Hyperlipoproteinemia Type IV; Retinoschisis; Nasopharyngeal Neoplasms; Adult-onset Still's Disease; Breakthrough Pain; Corneal Wavefront Aberration; Esophageal Squamous Cell Carcinoma; Oral Squamous Cell Carcinoma; Jackhammer Esophagus; Solitary Fibrous Tumors; Heat Stroke; Primary Liver Cancer; Metastatic Squamous Neck Cancer With Occult Primary; Urinary Fistula; Arthritis, Experimental; Child Nutrition Disorders; Donnai-Barrow Syndrome; Gait Apraxia; Peripheral T-cell Lymphoma; Lipid Metabolism Disorders; Temporomandibular Joint Dysfunction Syndrome; Hyperlipoproteinemia Type V; Retrocochlear Diseases; Nasopharyngitis; Adult-onset Vitelliform Macular Dystrophy; Breast Cancer Lymphedema; Cornelia De Lange Syndrome; Esophageal Stenosis; Oral Submucous Fibrosis; Jansen Type Metaphyseal Chondrodysplasia; Solitary Kidney; Heavy Metal Poisoning; Primary Myelofibrosis; Metatarsalgia; Urinary Incontinence; Arthritis, Gouty; CHILD Syndrome; Dopamine Beta Hydroxylase Deficiency; Gait Ataxia; Peripheral Vascular Diseases; Lipid Metabolism, Inborn Errors; Tendinopathy; Hyperlipoproteinemias; Retrognathia; Nausea; Affective Disorders, Psychotic; Breast Cancer, Male; Coronary Aneurysm; Esophageal Varices; Oral Ulcer; Japanese Encephalitis; Solitary Pulmonary Nodule; Heel Spur; Primary Orthostatic Hypotension; Metatarsus Varus; Urinary Incontinence, Stress; Arthritis, Infectious; Childhood Acute Lymphoblastic Leukemia; Double Outlet Right Ventricle; Gait Disorders, Neurologic; Periprosthetic Fractures; Lipidoses; Tendon Entrapment; Hypermobile Ehlers-Danlos Syndrome; Retroperitoneal Fibrosis; NBIA/DYT/PARK-PLA2G6; Affective Symptoms; Breast Carcinoma In Situ; Coronary Artery Aneurysm; Esophagitis; Orbital Cellulitis; Jaundice; Somatoform Disorders; Helicobacter Infections; Primary Orthostatic Tremor; Methemoglobinemia; Urinary Incontinence, Urge; Arthritis, Juvenile; Childhood Apraxia of Speech; Down Syndrome; Galactorrhea; Peritoneal Diseases; Lipodermatosclerosis; Tendon Injuries; Hypernatremia; Retroperitoneal Liposarcoma; Neck Injuries; Afibrinogenemia; Breast Cyst; Coronary Artery Disease; Esophagitis, Peptic; Orbital Diseases; Jaundice, Neonatal; Somatosensory Disorders; HELLP Syndrome; Primary Ovarian Insufficiency; Methemoglobinemia, Beta-globin Type; Urinary Retention; Arthritis, Psoriatic; Childhood Brain Stem Glioma; Doxorubicin Induced Cardiomyopathy; Galactorrhoea-Hyperprolactinaemia; Peritoneal Fibrosis; Lipodystrophy; Tennis Elbow; Hyperopia; Retroperitoneal Neoplasms; Neck Pain; Agammaglobulinemia; Breast Diseases; Coronary Disease; Esotropia; Orbital Fractures; Jaundice, Obstructive; Somatostatinoma; Helminthiasis; Primary Pigmented Nodular Adrenocortical Disease; Methionine Adenosyltransferase Deficiency; Urinary Tract Infections; Arthritis, Reactive; Childhood Disintegrative Disorder; Dravet Syndrome; Galactose Epimerase Deficiency; Peritoneal Neoplasms; Lipodystrophy Due to Peptidic Growth Factors Deficiency; Tenosynovitis; Hyperostosis; Retropharyngeal Abscess; Necrobiosis Lipoidica; Agenesis of Corpus Callosum; Breast Neoplasms; Coronary Occlusion; Essential Hypertension; Orbital Lymphoma; Jaw Abnormalities; Somnambulism; Hemangioblastoma; Primary Progressive Aphasia; Methylmalonic Acidemia; Urination Disorders; Arthritis, Rheumatoid; Childhood Hepatocellular Carcinoma; Drinking Behavior; Galactosemias; Peritonitis; Lipodystrophy, Congenital Generalized; Tension-Type Headache; Hyperostosis, Diffuse Idiopathic Skeletal; Retroviridae Infections; Necrobiotic Disorders; Ageusia; Breast Neoplasms, Male; Coronary Restenosis; Essential Thrombocythemia; Orbital Neoplasms; Jaw Diseases; Space Motion Sickness; Hemangioendothelioma; Primary Progressive Nonfluent Aphasia; Methylmalonic Acidemia With Homocystinuria Type cbIC; Urogenital Abnormalities; Arthrogryposis; Childhood Hypophosphatasia; Drowning; Galactosialidosis; Peritonitis, Tuberculous; Lipodystrophy, Familial Partial; Teratogenesis; Hyperoxaluria; Rett Syndrome; Necrosis; Aggression; Breech Presentation; Coronary Stenosis; Essential Tremor; Orbital Pseudotumor; Jaw Fractures; Spasm; Hemangioendothelioma, Epithelioid; Primary Sclerosing Cholangitis; Metrorrhagia; Urogenital Neoplasms; Arthrogryposis Multiplex Congenita; Childhood Supratentorial Embryonal Tumor, Not Otherwise Specified; Drug Eruptions; Gallbladder Cancer; Peritonsillar Abscess; Lipogranulomatosis; Teratoma; Hyperoxaluria, Primary; Reversible Cerebral Vasoconstriction Syndrome; Necrotizing Autoimmune Myopathy; Aggressive NK Cell Leukemia; Brenner Tumor; Coronary Thrombosis; Esthesioneuroblastoma, Olfactory; Orchitis; Jaw, Edentulous; Spasmodic Dysphonia; Hemangioma; Primary Spontaneous Pneumothorax; Mevalonate Kinase Deficiency; Urolithiasis; Arthropathy, Neurogenic; Childhood-Onset Fluency Disorder; Drug Hypersensitivity; Gallbladder Diseases; Perivascular Epithelioid Cell Neoplasms; Lipoma; Teratozoospermia; Hyperoxia; Revesz Syndrome; Necrotizing Enterocolitis; Aggressive Periodontitis; Brenner Tumor of Ovary; Coronary Vasospasm; Ethmoid Sinusitis; Organic Acidemia; Jaw, Edentulous, Partially; Spasms, Infantile; Hemangioma Thrombocytopenia Syndrome; Prinzmetal's Variant Angina; Mevalonic Aciduria; Urologic Diseases; Articulation Disorders; Childhood-Onset Schizophrenia; Drug Hypersensitivity Syndrome; Gallbladder Neoplasms; Periventricular Heterotopia; Lipomatosis; Testicular Cancer; Hyperparathyroidism; Rhabdoid Tumor; Necrotizing Fasciitis; Aging, Premature; Brittle Cornea Syndrome; Coronary Vessel Anomalies; Euglenozoa Infections; Organophosphate Poisoning; Jervell-Lange Nielsen Syndrome; Spastic Ataxia Charlevoix-Saguenay Type; Hemangioma, Capillary; Prion Diseases; Microaneurysm; Urologic Neoplasms; Arts Syndrome; Children's Interstitial Lung Disease; Drug Overdose; Gallstones; Periventricular Leukomalacia; Lipomatosis, Multiple Symmetrical; Testicular Diseases; Hyperparathyroidism, Primary; Rhabdomyolysis; Needlestick Injuries; Agnosia; Brittle Diabetes; Coronavirus Infections; Eunuchism; Ornithine Carbamoyltransferase Deficiency Disease; Jet Lag Syndrome; Spastic Diplegia Cerebral Palsy; Hemangioma, Cavernous; Problem Behavior; Microcephalic Osteodysplastic Primordial Dwarfism Type 1; Urological Manifestations; Asbestosis; Chlamydia Infections; Drug Reaction With Eosinophilia and Systemic Symptoms; Gambling; Periventricular Nodular Heterotopia; Lipomyelomeningocele; Testicular Germ Cell Tumor; Hyperparathyroidism, Secondary; Rhabdomyoma; Neglected Diseases; Agoraphobia; Broken Heart Syndrome; Corpus Callosum Agenesis; Euthyroid Sick Syndromes; Ornithine Transcarbamylase Deficiency; Jeune Syndrome; Spastic Paraparesis; Hemangioma, Cavernous, Central Nervous System; Proctitis; Microcephaly; Urticaria; Ascariasis; Chlamydial Pneumonia; Drug Resistant Epilepsy; Gangliocytoma; Permanent Neonatal Diabetes Mellitus; Liposarcoma; Testicular Hydrocele; Hyperphagia; Rhabdomyosarcoma; Neisseria meningitidis Infection; Agranulocytosis; Bronchial Adenomas/carcinoids Childhood; Corticobasal Degeneration; Evans Syndrome; Orofaciodigital Syndromes; JMP Syndrome; Spastic Paraplegia, Hereditary; Hemangiopericytoma; Proctocolitis; Microcystic Adnexal Carcinoma; Urticaria Pigmentosa; Ascaridiasis; Choanal Atresia; Drug-Related Side Effects and Adverse Reactions; Ganglioglioma; Perniosis; Liposarcoma, Myxoid; Testicular Neoplasms; Hyperphosphatemia; Rhabdomyosarcoma Alveolar; Neisseriaceae Infections; Agraphia; Bronchial Diseases; Corynebacterium Infections; Ewing Sarcoma; Oropharyngeal Cancer, Adult; Job Syndrome; Specific Antibody Deficiency; Hemangiosarcoma; Prodromal Symptoms; Microcystic Lymphatic Malformation; Usher Syndrome, Type 1; Ascites; Cholangiocarcinoma; Drug-Seeking Behavior; Ganglion Cysts; Peroneal Neuropathies; Listeria Infection; Testicular Seminoma; Hyperpigmentation; Rhabdomyosarcoma Embryonal; Nelson Syndrome; Aicardi Syndrome; Bronchial Fistula; Costello Syndrome; Ewing's Family of Tumors; Oropharyngeal Neoplasms; Joint Deformities, Acquired; Specific Learning Disorder; Hemarthrosis; Progeria; Microencephaly; Usher Syndrome, Type 1B; Ascorbic Acid Deficiency; Cholangitis; Dry Eye Syndromes; Ganglioneuroblastoma; Peroxisomal Biogenesis Disorders; Listeriosis; Testicular Yolk Sac Tumor; Hyperplasia; Rhabdomyosarcoma, Alveolar; Nemaline Myopathy; Aicardi-Goutieres Syndrome; Bronchial Hyperreactivity; Cough; Exanthema; Orotic Aciduria Type 1; Joint Diseases; Speech Disorders; Hematemesis; Prognathism; Micrognathism; Usher Syndromes; Asherman's Syndrome; Cholangitis, Sclerosing; Dry Socket; Ganglioneuroma; Peroxisomal Disorders; Lithiasis; Testotoxicosis; Hyperprolactinemia; Rhabdomyosarcoma, Embryonal; Nematode Infections; AIDS Dementia Complex; Bronchial Neoplasms; Cowden Syndrome; Exercise-induced Anaphylaxis; Orthomyxoviridae Infections; Joint Dislocations; Speech Sound Disorder; Hematologic Diseases; Progressive Bulbar Palsy; Microphthalmia; Uterine Carcinosarcoma; Aspartylglucosaminuria; Cholecystitis; Duane Retraction Syndrome; Gangliosidoses; Peroxisome Biogenesis disorder-Zellweger Syndrome Spectrum; Livedoid Vasculopathy; Tetanus; Hyperprolinemia; Rheumatic Diseases; Neointima; AIDS-Associated Nephropathy; Bronchial Spasm; Coxsackievirus Infections; Exfoliation Syndrome; Orthostatic Intolerance; Joint Instability; Spermatic Cord Torsion; Hematologic Neoplasms; Progressive Familial Intrahepatic Cholestasis 1; Microphthalmos; Uterine Cervical Diseases; Aspartylglycosaminuria; Cholecystitis, Acute; Duane Syndrome; Gangliosidoses, GM2; Persian Gulf Syndrome; Liver Abscess; Tetany; Hypersensitivity; Rheumatic Fever; Neonatal Abstinence Syndrome; AIDS-Related Complex; Bronchiectasis; Cramp-fasciculation Syndrome; Exfoliative Dermatitis; Ossification of Posterior Longitudinal Ligament; Joint Loose Bodies; Spermatocele; Hematoma; Progressive Hemifacial Atrophy; Microsatellite Instability; Uterine Cervical Dysplasia; Asperger Syndrome; Cholecystolithiasis; Duane-radial Ray Syndrome; Gangliosidosis, GM1; Persistent Fetal Circulation Syndrome; Liver Abscess, Amebic; Tethered Cord Syndrome; Hypersensitivity Pneumonitis; Rheumatic Heart Disease; Neonatal Adrenoleukodystrophy; AIDS-Related Opportunistic Infections; Bronchiolitis; Cranial Nerve Diseases; Exocrine Pancreatic Insufficiency; Ossification, Heterotopic; Joubert Syndrome; Sphenoid Sinusitis; Hematoma, Epidural, Cranial; Progressive Multifocal Leukoencephalopathy; Microscopic Polyangiitis; Uterine Cervical Incompetence; Aspergillosis; Choledochal Cyst; Duarte Galactosemia; Gangrene; Persistent Hyperplastic Primary Vitreous; Liver Abscess, Pyogenic; Tetrahydrobiopterin Deficiency; Hypersensitivity Vasculitis; Rheumatoid Nodule; Neonatal Brachial Plexus Palsy; Airway Obstruction; Bronchiolitis Obliterans; Craniocerebral Trauma; Exophthalmos; Osteitis; Joubert Syndrome With Oculorenal Anomalies; Spherocytosis, Hereditary; Hematoma, Epidural, Spinal; Progressive Myoclonic Epilepsy; Microsporidiosis; Uterine Cervical Neoplasms; Aspergillosis, Allergic Bronchopulmonary; Choledocholithiasis; Dubin-Johnson Syndrome; Gardner Syndrome; Persistent Truncus Arteriosus; Liver Cirrhosis; Tetralogy of Fallot; Hypersensitivity, Delayed; Rheumatoid Vasculitis; Neonatal Herpes; Airway Remodeling; Bronchiolitis Obliterans Organizing Pneumonia; Craniofacial Abnormalities; Exostoses; Osteitis Deformans; Junctional Epidermolysis Bullosa; Spheroid Body Myopathy; Hematoma, Subdural; Progressive Non-fluent Aphasia; Microstomia; Uterine Cervicitis; Asphyxia; Cholelithiasis; Duchenne Muscular Dystrophy; Gas Gangrene; Persistent Vegetative State; Liver Cirrhosis, Alcoholic; Tetrasomy; Hypersensitivity, Immediate; Rhinitis; Neonatal Meningitis; Akathisia, Drug-Induced; Bronchiolitis, Viral; Craniofacial Dysostosis; Exostoses, Multiple Hereditary; Osteoarthritis; Juvenile Dermatomyositis; Sphincter of Oddi Dysfunction; Hematoma, Subdural, Acute; Progressive Supranuclear Palsy; Microtia-Anotia; Uterine Diseases; Asphyxia Neonatorum; Cholera; Ductus Arteriosus, Patent; Gastric Antral Vascular Ectasia; Personality Disorders; Liver Cirrhosis, Biliary; Tetrasomy X; Hypersomnolence, Idiopathic; Rhinitis, Allergic; Neonatal Onset Multisystem Inflammatory Disease; Akinetic Mutism; Bronchitis; Craniofacial Fibrous Dysplasia; Exotropia; Osteoarthritis, Hip; Juvenile Huntington Disease; Sphingolipidoses; Hematoma, Subdural, Chronic; Progressive Supranuclear Palsy Atypical; Microvascular Angina; Uterine Hemorrhage; Asthenia; Cholestasis; Dumping Syndrome; Gastric Fistula; Peters Anomaly; Liver Cirrhosis, Experimental; Thalassemia; Hypersplenism; Rhinitis, Allergic, Perennial; Neonatal Ovarian Cyst; AL Amyloidosis; Bronchitis, Chronic; Craniofacial Microsomia; Exsanguination; Osteoarthritis, Knee; Juvenile Myelomonocytic Leukemia; Spider Bites; Hematoma, Subdural, Intracranial; Prolactinoma; Microvascular Rarefaction; Uterine Inertia; Asthenopia; Cholestasis, Extrahepatic; Duodenal Atresia; Gastric Lymphoma; Peutz-Jeghers Syndrome; Liver Diseases; Therapy Related Acute Myeloid Leukemia and Myelodysplastic Syndrome; Hypertelorism; Rhinitis, Allergic, Seasonal; Neonatal Sepsis; Alagille Syndrome; Bronchomalacia; Craniofrontonasal Dysplasia; Exstrophy of the Bladder; Osteoarthritis, Spine; Juvenile Myoclonic Epilepsy; Spina Bifida; Hematuria; Prolapse; Midline Lethal Granuloma; Uterine Neoplasms; Asthenozoospermia; Cholestasis, Intrahepatic; Duodenal Diseases; Gastric Outlet Obstruction; Pfeiffer Syndrome; Liver Diseases, Alcoholic; Thiamine Deficiency; Hypertension; Rhinitis, Atrophic; Neonatal Stroke; Albinism; Bronchopneumonia; Craniomandibular Disorders; Exstrophy-epispadias Complex; Osteoarthropathy, Primary Hypertrophic; Juvenile Osteoporosis; Spina Bifida Cystica; Hemianopsia; Prolidase Deficiency; Migraine Disorders; Uterine Perforation; Asthma; Cholesteatoma; Duodenal Neoplasms; Gastrinoma; PGM1-CDG; Liver Failure; Thinness; Hypertension, Malignant; Rhinitis, Vasomotor; Neonatal Systemic Lupus Erythematosus; Albinism, Ocular; Bronchopulmonary Dysplasia; Craniopharyngioma; Extensively Drug-Resistant Tuberculosis; Osteoarthropathy, Secondary Hypertrophic; Juvenile Polymyositis; Spina Bifida Occulta; Hemicrania Continua; Proliferative Verrucous Leukoplakia; Migraine with Aura; Uterine Prolapse; Asthma, Aspirin-Induced; Cholesteatoma, Middle Ear; Duodenal Obstruction; Gastritis; PHACE Syndrome; Liver Failure, Acute; Thomas Syndrome; Hypertension, Portal; Rhinoscleroma; Neoplasm Metastasis; Albinism, Oculocutaneous; Brown Syndrome; Craniosynostoses; Extragonadal Germ Cell Tumor; Osteoblastoma; Juvenile Polyposis Syndrome; Spinal Cord Compression; Hemifacial Microsomia; Proopiomelanocortin Deficiency; Migraine without Aura; Uterine Retroversion; Asthma, Exercise-Induced; Cholesterol Ester Storage Disease; Duodenal Ulcer; Gastritis, Atrophic; Phagocyte Bactericidal Dysfunction; Liver Neoplasms; Thoracic Diseases; Hypertension, Pregnancy-Induced; Rhizomelic Chondrodysplasia Punctata; Neoplasm Micrometastasis; Albuminuria; Brown-Sequard Syndrome; CREST Syndrome; Extramammary Paget Disease; Osteochondritis; Juvenile Retinoschisis; Spinal Cord Diseases; Hemifacial Spasm; Propionic Acidemia; Mild Phenylketonuria; Uterine Rupture; Asthma, Occupational; Cholesteryl Ester Storage Disease; Duodenitis; Gastritis, Hypertrophic; Phantom Limb; Liver Neoplasms, Experimental; Thoracic Injuries; Hypertension, Pulmonary; RHYNS Syndrome; Neoplasm Recurrence, Local; Alcohol Abstinence; Brucellosis; Creutzfeldt-Jakob Disease; Extranodal Nasal NK/T Cell Lymphoma; Osteochondritis Dissecans; Juvenile Spondyloarthropathy; Spinal Cord Injuries; Hemihypertrophy; Propofol Infusion Syndrome; Milk Hypersensitivity; Uterine Sarcoma; Astigmatism; Choline Deficiency; Duodenogastric Reflux; Gastro-enteropancreatic Neuroendocrine Tumor; Pharyngeal Diseases; Localized Lipodystrophy; Thoracic Neoplasms; Hypertension, Renal; Rib Fractures; Neoplasm Regression, Spontaneous; Alcohol Amnestic Disorder; Brugada Syndrome; Creutzfeldt-Jakob Syndrome; Extravasation of Diagnostic and Therapeutic Materials; Osteochondrodysplasias; Kabuki Syndrome; Spinal Cord Ischemia; Hemiplegia; Prosopagnosia; Miller Fisher Syndrome; Uveal Diseases; Astrocytoma; Chondroblastoma; Dupuytren Contracture; Gastroenteritis; Pharyngeal Neoplasms; Localized Scleroderma; Thoracic Outlet Syndrome; Hypertension, Renovascular; Richter Syndrome; Neoplasm Seeding; Alcohol Drinking; Bruxism; Cri-du-Chat Syndrome; Eye Abnormalities; Osteochondroma; Kallmann Syndrome; Spinal Cord Neoplasms; Hemiplegic Migraine; Prostatic Diseases; Miller Syndrome; Uveal Neoplasms; Asymptomatic Diseases; Chondrocalcinosis; Dwarfism; Gastroesophageal Reflux; Pharyngitis; Locked-in Syndrome; Thrombasthenia; Hypertensive Retinopathy; Rickets; Neoplasm, Residual; Alcohol Drinking in College; Budd-Chiari Syndrome; Crigler Najjar Syndrome, Type 1; Eye Burns; Osteochondromatosis; Kaposi Sarcoma; Spinal Cord Vascular Diseases; Hemobilia; Prostatic Hyperplasia; Milroy Disease; Uveitis; Asymptomatic Infections; Chondrodysplasia Punctata; Dwarfism, Pituitary; Gastrointestinal Diseases; Phenylketonurias; Loeys-Dietz Syndrome; Thromboangiitis Obliterans; Hyperthyroidism; Rickets, Hypophosphatemic; Neoplasms by Histologic Type; Alcohol Withdrawal Delirium; Buerger Disease; Crigler-Najjar Syndrome; Eye Diseases; Osteochondrosis; Kaposi Varicelliform Eruption; Spinal Curvatures; Hemochromatosis; Prostatic Intraepithelial Neoplasia; Mineralocorticoid Excess Syndrome, Apparent; Uveitis, Anterior; Ataxia; Chondrodysplasia Punctata Syndrome; Dysarthria; Gastrointestinal Hemorrhage; Pheochromocytoma; Logopenic Progressive Aphasia; Thrombocythemia, Essential; Hypertrichosis; Rickettsia Infections; Neoplasms, Adipose Tissue; Alcohol Withdrawal Seizures; Bulbar Palsy, Progressive; Critical Illness; Eye Diseases, Hereditary; Osteogenesis Imperfecta; Kaposiform Hemangioendothelioma; Spinal Diseases; Hemochromatosis Type 3; Prostatic Neoplasms; Minicore Myopathy With External Ophthalmoplegia; Uveitis, Intermediate; Ataxia-Hypogonadism-Choroidal Dystrophy; Chondrodysplasia Punctata, Rhizomelic; Dysautonomia, Familial; Gastrointestinal Neoplasms; Philadelphia Chromosome; Loiasis; Thrombocytopenia; Hypertriglyceridemia; Rift Valley Fever; Neoplasms, Adnexal and Skin Appendage; Alcohol-Induced Disorders; Bulbo-Spinal Atrophy, X-Linked; Crohn Disease; Eye Foreign Bodies; Osteogenesis Imperfecta Type II; Kaposiform Lymphangiomatosis; Spinal Dysraphism; Hemochromatosis Type 4; Prostatic Neoplasms, Castration-Resistant; Minimal Change Disease; Uveitis, Posterior; Ataxia Telangiectasia; Chondroma; Dysbiosis; Gastrointestinal Stromal Tumors; Phimosis; Long QT Syndrome; Thrombocytopenia, Neonatal Alloimmune; Hypertriglyceridemic Waist; Rigid Spine Syndrome; Neoplasms, Basal Cell; Alcohol-Induced Disorders, Nervous System; Bulimia; Cross Infection; Eye Hemorrhage; Osteogenesis Imperfecta Type III; Kartagener Syndrome; Spinal Fractures; Hemoglobin C Disease; Prostatism; Miosis; Uveomeningoencephalitic Syndrome; Ataxia With Oculomotor Apraxia Type 1; Chondromalacia Patellae; Dyscalculia; Gastroparesis; Phlebitis; Long QT Syndrome 1; Thrombocytosis; Hypertrophy; RNA Virus Infections; Neoplasms, Bone Tissue; Alcohol-Related Disorders; Bulimia Nervosa; Croup; Eye Infections; Osteogenesis Imperfecta Type IV; Kasabach-Merritt Syndrome; Spinal Injuries; Hemoglobin SC Disease; Prostatitis; Mite Infestations; Vaccinia; Ataxia With Oculomotor Apraxia Type 2; Chondrosarcoma; Dysembryoplastic Neuroepithelial Tumor; Gastroschisis; Phobia, Social; Long QT Syndrome 3; Thromboembolism; Hypertrophy, Left Ventricular; ROHHAD; Neoplasms, Connective and Soft Tissue; Alcoholic Intoxication; Bullous Pemphigoid; Crouzon Syndrome; Eye Infections, Bacterial; Osteogenesis Imperfecta Type VI; Kashin-Beck Disease; Spinal Muscular Atrophies of Childhood; Hemoglobinemia; Prosthesis Failure; Mitochondrial Complex II Deficiency; VACTERL Association; Ataxia With Vitamin E Deficiency; Chondrosarcoma, Clear Cell; Dysentery; GATA2 Deficiency; Phobic Disorders; Long Term Adverse Effects; Thrombophilia; Hypertrophy, Right Ventricular; Rokitansky Sequence; Neoplasms, Connective Tissue; Alcoholic Korsakoff Syndrome; Bullying; Crush Injuries; Eye Infections, Fungal; Osteolysis; Kawasaki Disease; Spinal Muscular Atrophy; Hemoglobinopathies; Prosthesis-Related Infections; Mitochondrial Diseases; Vagina, Absence of; Athabaskan Brainstem Dysgenesis; Chondrosarcoma, Mesenchymal; Dysentery, Amebic; Gaucher Disease; Phosphoglycerate Kinase Deficiency; Lordosis; Thrombophlebitis; Hyperuricemia; Romano-Ward Syndrome; Neoplasms, Cystic, Mucinous, and Serous; Alcoholic Neuropathy; Bundle-Branch Block; Crusted Scabies; Eye Injuries; Osteolysis, Essential; Kearns-Sayre Syndrome; Spinal Muscular Atrophy 1; Hemoglobinuria; Prosthetic Joint Infection; Mitochondrial DNA Depletion Syndrome; Vaginal Cancer; Atherosclerosis; Chordoid Glioma of the Third Ventricle; Dysentery, Bacillary; Gaucher Disease Type 1; Phosphoglycerate Mutase Deficiency; Low Back Pain; Thrombosis; Hyperventilation; Root Caries; Neoplasms, Experimental; Alcoholism; Bunion; Cryoglobulinemia; Eye Injuries, Penetrating; Osteoma; Keloid; Spinal Muscular Atrophy Type 2; Hemoglobinuria, Paroxysmal; Protein C Deficiency; Mitochondrial DNA-associated Leigh Syndrome; Vaginal Discharge; Athletic Injuries; Chordoma; Dysequilibrium Syndrome; Gaucher Disease Type 2; Phosphorus Metabolism Disorders; Low Tension Glaucoma; Thrombotic Microangiopathies; Hypervitaminosis A; Root Resorption; Neoplasms, Fibrous Tissue; Alexander Disease; Burkholderia Infections; Cryopyrin-Associated Periodic Syndromes; Eye Manifestations; Osteoma, Osteoid; Kennedy Disease; Spinal Muscular Atrophy Type 3; Hemolysis; Protein Deficiency; Mitochondrial Encephalomyopathies; Vaginal Diseases; Atransferrinemia; Chorea; Dysferlinopathy; Gaucher Disease Type 3; Photophobia; Lowe Oculocerebrorenal Syndrome; Thrombotic Thrombocytopeniaurpura, Acquired; Hypesthesia; Rosacea; Neoplasms, Germ Cell and Embryonal; Alkalosis; Burkitt Lymphoma; Cryptococcosis; Eye Neoplasms; Osteomalacia; Keratitis; Spinal Muscular Atrophy With Respiratory Distress 1; Hemolytic-Uremic Syndrome; Protein-Energy Malnutrition; Mitochondrial Encephalomyopathy Lactic Acidosis and Stroke-like Episodes; Vaginal Fistula; Atrial Fibrillation; Chorea-acanthocytosis; Dysfibrinogenemia; Gender Dysphoria; Photosensitive Epilepsy; Lower Extremity Deformities, Congenital; Thymic Epithelial Tumor; Hypoadrenalism; Rosai-Dorfman Disease; Neoplasms, Glandular and Epithelial; Alkalosis, Respiratory; Burning Mouth Syndrome; Cryptogenic Organizing Pneumonia; Eye Pain; Osteomyelitis; Keratitis, Herpetic; Spinal Neoplasms; Hemoperitoneum; Protein-Losing Enteropathies; Mitochondrial Genetic Disorders; Vaginal Neoplasms; Atrial Flutter; Chorioamnionitis; Dysgerminoma; Generalized Pustular Psoriasis; Photosensitivity Disorders; Lower Urinary Tract Symptoms; Thymoma; Hypoadrenocorticism, Familial; Rotator Cuff Injuries; Neoplasms, Hormone-Dependent; Alkaptonuria; Burnout, Professional; Cryptorchidism; Eyelid Diseases; Osteonecrosis; Keratoacanthoma; Spinal Osteochondrosis; Hemophagocytic Lymphohistiocytosis; Proteinuria; Mitochondrial Myopathies; Vaginismus; Atrial Myxoma, Familial; Choriocarcinoma; Dysgeusia; Genetic Diseases, Inborn; Phyllodes Tumor; Lung Abscess; Thymus Neoplasms; Hypoalbuminemia; Rotator Cuff Tear Arthropathy; Neoplasms, Mesothelial; Allan-Herndon-Dudley Syndrome; Burnout, Psychological; Cryptosporidiosis; Fabry Disease; Osteopetrosis; Keratoconjunctivitis; Spinal Shock; Hemophilia; Proteus Infections; Mitochondrial Neurogastrointestinal Encephalopathy Syndrome; Vaginitis; Atrial Premature Complexes; Chorioretinitis; Dyskeratosis Congenita; Genetic Diseases, X-Linked; Phyllodes Tumor of the Breast; Lung Adenocarcinoma; Thyroid Cancer, Follicular; Hypoaldosteronism; Rotavirus Infections; Neoplasms, Multiple Primary; Allergic Bronchopulmonary Aspergillosis; Burns; Crystal Arthropathies; FACES Syndrome; Osteopetrosis Autosomal Dominant Type 2; Keratoconjunctivitis Sicca; Spinal Stenosis; Hemophilia A; Proteus Syndrome; Mitochondrial Trifunctional Protein Deficiency; Vaginosis, Bacterial; Atrial Remodeling; Choristoma; Dyskinesia, Drug-Induced; Genetic Predisposition to Disease; Pica; Lung Diseases; Thyroid Cancer, Medullary; Hypoalphalipoproteinemias; Rothmund-Thomson Syndrome; Neoplasms, Nerve Tissue; Alopecia; Burns, Chemical; Cubital Tunnel Syndrome; Facial Asymmetry; Osteophyte; Keratoconus; Spinocerebellar Ataxia 1; Hemophilia B; Prothrombin Deficiency; Mitral Valve Insufficiency; Vagus Nerve Diseases; Atrioventricular Block; Choroid Diseases; Dyskinesias; Genital Diseases, Female; Pick Disease of the Brain; Lung Diseases, Fungal; Thyroid Cancer, Papillary; Hypobetalipoproteinemia, Familial, Apolipoprotein B; Roussy Levy Syndrome; Neoplasms, Neuroepithelial; Alopecia Areata; Burns, Electric; Cumulative Trauma Disorders; Facial Dermatoses; Osteoporosis; Keratoderma, Palmoplantar; Spinocerebellar Ataxia 10; Hemophilic Arthropathy; Protoporphyria; Mitral Valve Prolapse; Vagus Nerve Injuries; Atrioventricular Septal Defect; Choroid Hemorrhage; Dyslexia; Genital Diseases, Male; Picornaviridae Infections; Lung Diseases, Interstitial; Thyroid Carcinoma, Anaplastic; Hypobetalipoproteinemias; Rubella; Neoplasms, Plasma Cell; Alopecia Totalis; Bursitis; Currarino Triad; Facial Hemiatrophy; Osteoporosis, Postmenopausal; Keratoderma, Palmoplantar, Diffuse; Spinocerebellar Ataxia 11; Hemopneumothorax; Protoporphyria, Erythropoietic; Mitral Valve Stenosis; Varicella Zoster Virus Infection; Atrophic Vaginitis; Choroid Neoplasms; Dyslexia, Acquired; Genital Neoplasms, Female; Piebaldism; Lung Diseases, Obstructive; Thyroid Diseases; Hypocalcemia; Rubella Syndrome, Congenital; Neoplasms, Radiation-Induced; Alopecia Universalis; Buruli Ulcer; Cushing Syndrome; Facial Injuries; Osteoporosis-pseudoglioma Syndrome; Keratomalacia; Spinocerebellar Ataxia 12; Hemoptysis; Protozoan Infections; Mixed Connective Tissue Disease; Varicocele; Atrophy; Choroid Plexus Carcinoma; Dyslipidemias; Genital Neoplasms, Male; Pierre Robin Sequence; Lung Injury; Thyroid Dysgenesis; Hypocapnia; Rubinstein-Taybi Syndrome; Neoplasms, Second Primary; Alpers Syndrome; Cachexia; Cutaneous Fistula; Facial Neoplasms; Osteoporotic Fractures; Keratosis; Spinocerebellar Ataxia 13; Hemorrhage; Prurigo; Mixed Tumor, Malignant; Varicose Ulcer; Attention Deficit and Disruptive Behavior Disorders; Choroid Plexus Neoplasms; Dysmenorrhea; Genomic Instability; Pierre Robin Syndrome; Lung Neoplasms; Thyroid Neoplasms; Hypochondriasis; Rumination Disorder; Neoplasms, Squamous Cell; Alpha 1-Antitrypsin Deficiency; CADASIL; Cutaneous Lupus Erythematosus; Facial Nerve Diseases; Osteoradionecrosis; Keratosis, Actinic; Spinocerebellar Ataxia 14; Hemorrhagic Disorders; Prurigo Nodularis; Mixed Tumor, Mesodermal; Varicose Veins; Attention Deficit Disorder with Hyperactivity; Choroidal Neovascularization; Dysostoses; Genu Valgum; Pigment-dispersion Syndrome; Lung, Hyperlucent; Thyroid Nodule; Hypochondroplasia; Rupture; Neoplasms, Unknown Primary; alpha-Mannosidosis; Cadaver; Cutaneous Mastocytosis; Facial Nerve Injuries; Osteosarcoma; Keratosis, Seborrheic; Spinocerebellar Ataxia 15; Hemorrhagic Fever with Renal Syndrome; Pruritus; Mixed Tumor, Mullerian; Variegate Porphyria; Attenuated Familial Adenomatous Polyposis; Choroideremia; Dyspareunia; Genu Varum; Pigmentation Disorders; Lupus Erythematosus, Cutaneous; Thyroiditis; Hypocomplementemic Urticarial Vasculitis; Rupture, Spontaneous; Neoplasms, Vascular Tissue; alpha-Thalassemia; Calcinosis; Cutaneous Polyarteritis Nodosa; Facial Neuralgia; Osteosarcoma, Juxtacortical; Kernicterus; Spinocerebellar Ataxia 17; Hemorrhagic Fever, Crimean; Pruritus Ani; Miyoshi Myopathy; Vasa Previa; Atypical Hemolytic Uremic Syndrome; Choroiditis; Dyspepsia; Geographic Atrophy; Pigmented Villonodular Synovitis; Lupus Erythematosus, Discoid; Thyroiditis, Autoimmune; Hypoglossal Nerve Diseases; Russell-Silver Syndrome; Neoplastic Cells, Circulating; Alphavirus Infections; Calciphylaxis; Cutaneous Sclerosis; Facial Pain; Osteosclerosis; Ketosis; Spinocerebellar Ataxia 18; Hemorrhagic Fever, Ebola; Pruritus Vulvae; Mobility Limitation; Vascular Calcification; Atypical Squamous Cells of the Cervix; Chromophil Renal Cell Carcinoma; Dysphonia; Germ Cells Tumors; PIK3CA-related Overgrowth Spectrum; Lupus Erythematosus, Systemic; Thyroiditis, Subacute; Hypoglycemia; Sacrococcygeal Teratoma; Neoplastic Processes; Alport Syndrome; Calcium Metabolism Disorders; Cutaneous T-cell Lymphoma; Facial Paralysis; Ostium Secundum Atrial Septal Defect; Kidney Calculi; Spinocerebellar Ataxia 2; Hemorrhagic Fevers, Viral; Pseudarthrosis; Mobius Syndrome; Vascular Diseases; Auditory Diseases, Central; Chromophobe Renal Cell Carcinoma; Dysplastic Nevus Syndrome; Germinoma; Pilocytic Astrocytoma; Lupus Nephritis; Thyrotoxic Periodic Paralysis; Hypogonadism; Sacroiliitis; Neoplastic Syndromes, Hereditary; AlstrÃIm Syndrome; Calculi; Cutaneous-skeletal Hypophosphatemia Syndrome; Facies; Otitis; Kidney Diseases; Spinocerebellar Ataxia 20; Hemorrhoids; Pseudoachondroplasia; Molluscum Contagiosum; Vascular Ehlers-Danlos Syndrome; Auditory Neuropathy Spectrum Disorder; Chromosomal Instability; Dyspnea; Gestational Trophoblastic Disease; Pilomatrixoma; Lupus Vasculitis, Central Nervous System; Thyrotoxicosis; Hypohidrosis; Sagittal Sinus Thrombosis; Neovascularization, Pathologic; Alstrom Syndrome; Caliciviridae Infections; Cutis Laxa; Facioscapulohumeral Muscular Dystrophy; Otitis Externa; Kidney Diseases, Cystic; Spinocerebellar Ataxia 21; Hemosiderosis; Pseudocholinesterase Deficiency; Molybdenum Cofactor Deficiency; Vascular Fistula; Auditory Perceptual Disorders; Chromosomal Triplication; Dyspnea, Paroxysmal; Gestational Trophoblastic Tumor; Pilonidal Sinus; Lyme Disease; Tibial Fractures; Hypohidrotic Ectodermal Dysplasia; Salivary Gland Diseases; Nephritis; Alternating Hemiplegia of Childhood; Callosities; Cyanosis; Factor V Deficiency; Otitis Media; Kidney Failure, Chronic; Spinocerebellar Ataxia 23; Hemospermia; Pseudohypoaldosteronism; Monckeberg Medial Calcific Sclerosis; Vascular Headaches; Autism Spectrum Disorder; Chromosome 15q Duplication; Dyssomnias; Gestational Weight Gain; Pineal Germ Cell Tumor; Lyme Neuroborreliosis; Tibial Meniscus Injuries; Hypokalemia; Salivary Gland Neoplasms; Nephritis, Hereditary; Altitude Sickness; Campylobacter Infections; Cyberbullying; Factor VII Deficiency; Otitis Media with Effusion; Kidney Neoplasms; Spinocerebellar Ataxia 25; Hemostatic Disorders; Pseudohypoparathyroidism; Mondini Dysplasia; Vascular Malformations; Autistic Disorder; Chromosome 17p Deletion; Dysthymic Disorder; Giant Axonal Neuropathy; Pineal Parenchymal Tumors of Intermediate Differentiation; Lymph Node Neoplasm; Tibial Neuropathy; Hypokalemic Periodic Paralysis; Salmonella Infections; Nephritis, Interstitial; Alveolar Bone Loss; Camurati-Engelmann Disease; Cyclothymic Disorder; Factor X Deficiency; Otitis Media, Suppurative; Kidney Tubular Necrosis, Acute; Spinocerebellar Ataxia 26; Hemothorax; Pseudohypoparathyroidism Type 1A; Monkeypox; Vascular Neoplasms; Autoimmune Autonomic Ganglionopathy; Chromosome 1p36 Deletion Syndrome; Dystocia; Giant Cell Arteritis; Pinealoma; Lymphadenitis; Tic Disorders; Hypokinesia; Salpingitis; Nephrocalcinosis; Alveolar Echinococcosis; Canavan Disease; Cylindroma; Factor XI Deficiency; Oto-Palatal-digital Syndrome; Kienbock's Disease; Spinocerebellar Ataxia 27; Henipavirus Infections; Pseudohypoparathyroidism With Albright Hereditary Osteodystrophy; Monoclonal Gammopathy of Undetermined Significance; Vascular Remodeling; Autoimmune Diseases; Chromosome 3q29 Microduplication Syndrome; Dystonia; Giant Cell Myocarditis; Pineoblastoma; Lymphadenopathy; Tick Bites; Hypolipoproteinemias; Salter-Harris Fractures; Nephrogenic Diabetes Insipidus; Alveolar Soft Part Sarcoma; Cancer Pain; Cystadenocarcinoma; Factor XII Deficiency; Oto-palato-digital Syndrome Type 1; Kikuchi Disease; Spinocerebellar Ataxia 28; Henoch-Schonlein Purpura; Pseudolymphoma; Monomelic Amyotrophy; Vascular Ring; Autoimmune Diseases of the Nervous System; Chromosome 5q Deletion; Dystonia Musculorum Deformans; Giant Cell Tumor of Bone; Pineocytoma; Lymphangiectasis; Tick-Borne Diseases; Hyponatremia; Sandhoff Disease; Nephrogenic Fibrosing Dermopathy; Alveolitis, Extrinsic Allergic; Candida Glabrata; Cystadenocarcinoma, Mucinous; Factor XIII Deficiency; Otomycosis; Klatskin Tumor; Spinocerebellar Ataxia 29; Hepadnaviridae Infections; Pseudomonas Infections; Mononeuropathies; Vascular System Injuries; Autoimmune Encephalitis; Chromosome 7q Duplication; Dystonic Disorders; Giant Cell Tumor of Tendon Sheath; Pinta; Lymphangiectasis, Intestinal; Tick-borne Encephalitis; Hypoparathyroidism; SAPHO Syndrome; Nephrogenic Systemic Fibrosis; Alzheimer Disease; Candidemia; Cystadenocarcinoma, Serous; Failed Back Surgery Syndrome; Otorhinolaryngologic Diseases; Klebsiella Infections; Spinocerebellar Ataxia 3; Heparin-induced Thrombocytopenia; Pseudomyxoma Peritonei; Monosomy; Vasculitis; Autoimmune Enteropathy; Chromosome Aberrations; Dystrophic Epidermolysis Bullosa; Giant Cell Tumors; Piriformis Muscle Syndrome; Lymphangioleiomyomatosis; Tics; Hypopharyngeal Cancer; Sarcoglycanopathies; Nephrolithiasis; Alzheimer Disease Type 1; Candidiasis; Cystadenoma; Failure to Thrive; Otorhinolaryngologic Neoplasms; Kleine-Levin Syndrome; Spinocerebellar Ataxia 30; Hepatic Encephalopathy; Pseudoneonatal Adrenoleukodystrophy; Mood Disorders; Vasculitis, Central Nervous System; Autoimmune Hemolytic Anemia; Chromosome Deletion; Dystrophinopathy; Giant Papillary Conjunctivitis; Piriformis Syndrome; Lymphangioma; Tinea; Hypopharyngeal Neoplasms; Sarcoidosis; Nephroma, Mesoblastic; Alzheimer Disease Type 2; Candidiasis, Chronic Mucocutaneous; Cystadenoma, Mucinous; Fallopian Tube Cancer; Otosclerosis; Klinefelter Syndrome; Spinocerebellar Ataxia 31; Hepatic Insufficiency; Pseudopelade of Brocq; Morning Sickness; Vasculitis, Leukocytoclastic, Cutaneous; Autoimmune Hepatitis; Chromosome Disorders; Dysuria; Giardiasis; Pitt-Hopkins Syndrome; Lymphangiomatosis; Tinea Capitis; Hypophosphatasia; Sarcoidosis, Pulmonary; Nephronophthisis; Alzheimer Disease Type 3; Candidiasis, Invasive; Cystadenoma, Serous; Fallopian Tube Diseases; Out-of-Hospital Cardiac Arrest; Klippel-Feil Syndrome; Spinocerebellar Ataxia 34; Hepatic Veno-Occlusive Disease; Pseudophakia; Morphea; Vasoplegia; Autoimmune Inner Ear Disease; Chronic Active Epstein-Barr Virus Infection; Eales Disease; Gigantism; Pituitary ACTH Hypersecretion; Lymphangiomyoma; Tinea Pedis; Hypophosphatemia; Sarcoma; Nephropathic Cystinosis; Amaurosis Fugax; Candidiasis, Oral; Cystic Adenomatoid Malformation of Lung; Fallopian Tube Neoplasms; Ovarian Cancer; Klippel-Trenaunay Syndrome; Spinocerebellar Ataxia 35; Hepatitis; Pseudopseudohypoparathyroidism; Morphine Dependence; Vasospasm, Intracranial; Autoimmune Lymphoproliferative Syndrome; Chronic Disease; Ear Deformities, Acquired; Gigantomastia; Pituitary Cancer; Lymphangiosarcoma; Tinea Versicolor; Hypophosphatemia, Familial; Sarcoma Botryoides; Nephrosclerosis; Amblyopia; Candidiasis, Vulvovaginal; Cystic Adenomatoid Malformation of Lung, Congenital; Familial Adenomatous Polyposis; Ovarian Carcinosarcoma; Klippel-Trenaunay-Weber Syndrome; Spinocerebellar Ataxia 36; Hepatitis A; Pseudotumor Cerebri; Morton Neuroma; Vein of Galen Aneurysm; Autoimmune Pancreatitis; Chronic Eosinophilic Pneumonia; Ear Diseases; Gilbert Disease; Pituitary Diseases; Lymphatic Abnormalities; Tinnitus; Hypophosphatemic Rickets; Sarcoma, Alveolar Soft Part; Nephrosis; Amebiasis; CANDLE Syndrome; Cystic Fibrosis; Familial Alzheimer Disease; Ovarian Cysts; Knee Dislocation; Spinocerebellar Ataxia 37; Hepatitis B; Pseudoxanthoma Elasticum; Morvan's Fibrillary Chorea; Vein of Galen Malformations; Autoimmune Polyglandular Syndrome Type 1; Chronic Erosive Gastritis; Ear Neoplasms; Gingival Diseases; Pituitary Neoplasms; Lymphatic Diseases; Tissue Adhesions; Hypopigmentation; Sarcoma, Clear Cell; Nephrosis, Lipoid; Ameloblastoma; Capillary Hemangioblastoma; Cysticercosis; Familial Atrial Fibrillation; Ovarian Diseases; Knee Injuries; Spinocerebellar Ataxia 4; Hepatitis B, Chronic; Psoriasis; Motion Sickness; Velopharyngeal Insufficiency; Autoimmune Pulmonary Alveolar Proteinosis; Chronic Graft Versus Host Disease; Earache; Gingival Hemorrhage; Pituitary Stalk Interruption Syndrome; Lymphatic Filariasis; Tobacco Use Disorder; Hypopituitarism; Sarcoma, Endometrial Stromal; Nephrotic Syndrome; Amelogenesis Imperfecta; Capillary Leak Syndrome; Cystinosis; Familial Cerebral Cavernous Malformation; Ovarian Epithelial Cancer; Korsakoff Syndrome; Spinocerebellar Ataxia 5; Hepatitis C; Psoriatic Juvenile Idiopathic Arthritis; Motor Disorders; Venezuelan Equine Encephalitis; Autoimmune Retinopathy; Chronic Granulomatous Disease; Early Infantile Epileptic Encephalopathy; Gingival Hyperplasia; Pityriasis; Lymphatic Malformations; Togaviridae Infections; Hypoplastic Left Heart Syndrome; Sarcoma, Ewing; Nerve Compression Syndromes; Amenorrhea; Capsule Opacification; Cystinuria; Familial Cold Autoinflammatory Syndrome; Ovarian Germ Cell Tumor; Krabbe Disease; Spinocerebellar Ataxia 7; Hepatitis C, Chronic; Psychological Trauma; Motor Neuron Disease; Venous Insufficiency; Autonomic Dysreflexia; Chronic Inflammatory Demyelinating Polyneuropathy; Early-onset, Autosomal Dominant Alzheimer Disease; Gingival Hypertrophy; Pityriasis Lichenoides; Lymphatic Metastasis; Tongue Cancer; Hypoplastic Right Heart Syndrome; Sarcoma, Kaposi; Nerve Degeneration; Amino Acid Metabolism, Inborn Errors; Carbamoyl Phosphate Synthetase 1 Deficiency; Cystitis; Familial Colorectal Cancer; Ovarian Hyperstimulation Syndrome; Krukenberg Carcinoma; Spinocerebellar Ataxia 8; Hepatitis D; Psychomotor Agitation; Motor Skills Disorders; Venous Thromboembolism; Autonomic Nervous System Diseases; Chronic Intestinal Pseudoobstruction; Eastern Equine Encephalitis; Gingival Neoplasms; Pityriasis Lichenoides Chronica; Lymphedema; Tongue Diseases; Hypoproteinemia; Sarcoma, Myeloid; Nerve Sheath Neoplasms; AML With Myelodysplasia-related Features; Carbamoyl-Phosphate Synthase I Deficiency Disease; Cystitis, Interstitial; Familial Deafness; Ovarian Low Malignant Potential Tumor; Krukenberg Tumor; Spinocerebellar Ataxia Autosomal Recessive 3; Hepatitis D, Chronic; Psychomotor Disorders; Mounier-Kuhn Syndrome; Venous Thrombosis; Autosomal Dominant Cerebellar Ataxia; Chronic Kidney Disease-Mineral and Bone Disorder; Ebola Virus Disease; Gingival Overgrowth; Pityriasis Rosea; Lymphoblastic Lymphoma; Tongue Neoplasms; Hypoprothrombinemias; Sarcoma, Synovial; Nervous System Malformations; Amnesia; Carbohydrate Metabolism, Inborn Errors; Cystocele; Familial Dilated Cardiomyopathy; Ovarian Neoplasms; KSHV Inflammatory Cytokine Syndrome; Spinocerebellar Ataxia Autosomal Recessive 7; Hepatitis E; Psychophysiologic Disorders; Mouth Abnormalities; Ventilator-Induced Lung Injury; Autosomal Dominant Cerebellar Ataxia, Deafness, and Narcolepsy; Chronic Lymphocytic Leukemia; Ebstein Anomaly; Gingival Pocket; Pityriasis Rubra Pilaris; Lymphocele; Tonsillar Neoplasms; Hypospadias; Sarcopenia; Nervous System Neoplasms; Amnesia, Transient Global; Carbon Monoxide Poisoning; Cysts; Familial Dysautonomia; Ovarian Sex Cord Tumor With Annular Tubules; Kuster Syndrome; Spinocerebellar Ataxia Autosomal Recessive With Axonal Neuropathy; Hepatitis, Alcoholic; Psychoses, Substance-Induced; Mouth Breathing; Ventricular Dysfunction; Autosomal Dominant Hyper IgE Syndrome; Chronic Myeloid Leukemia; Ecchymosis; Gingival Recession; Placenta Accreta; Lymphocytosis; Scabies; Nesidioblastosis; Amphetamine-Related Disorders; Carbuncle; Cytomegalic Inclusion Disease; Familial Encephalopathy With Neuroserpin Inclusion Bodies; Ovarian Small Cell Carcinoma; Kwashiorkor; Spinocerebellar Ataxia Type 6; Hepatitis, Autoimmune; Psychotic Disorders; Mouth Diseases; Ventricular Dysfunction, Left; Autosomal Dominant Partial Epilepsy With Auditory Features; Chronic Myelomonocytic Leukemia; Eccrine Porocarcinoma; Gingivitis; Placenta Diseases; Lymphohistiocytosis, Hemophagocytic; Tooth Abnormalities; Hypotension, Orthostatic; Scheie Syndrome; Netherton Syndrome; Amputation, Traumatic; Carcinogenesis; Cytomegalovirus Infections; Familial Exudative Vitreoretinopathy; Overbite; Kyphosis; Spinocerebellar Ataxias; Hepatitis, Chronic; PTEN Hamartoma Tumor Syndrome; Mouth Neoplasms; Ventricular Dysfunction, Right; Autosomal Dominant Tubulointerstitial Kidney Disease; Chronic Myeloproliferative Disorders; Echinococcosis; Gingivitis, Necrotizing Ulcerative; Placenta Disorder; Lymphoma; Tooth Abrasion; Hypothalamic Diseases; Scheuermann Disease; Neural Crest Tumor; Amyloid Neuropathies; Carcinoid Heart Disease; Cytomegalovirus Retinitis; Familial HDL Deficiency; Overnutrition; Labor Pain; Spinocerebellar Degenerations; Hepatitis, Viral, Human; Pterygium; Mouth, Edentulous; Ventricular Fibrillation; Autosomal Recessive Axonal Neuropathy With Neuromyotonia; Chronic Neutrophilic Leukemia; Echinococcosis, Hepatic; Gitelman Syndrome; Placenta Previa; Lymphoma AIDS Related; Tooth Ankylosis; Hypothalamic Neoplasms; Schistosomiasis; Neural Tube Defects; Amyloid Neuropathies, Familial; Carcinoid Syndrome; D-bifunctional Protein Deficiency; Familial Hemiplegic Migraine; Overweight; Labyrinth Diseases; Splenic Rupture; Hepatoblastoma; Pterygium of the Conjunctiva and Cornea; Movement Disorders; Ventricular Flutter; Autosomal Recessive Hyper IgE Syndrome; Chronic Pain; Eclampsia; Glanders; Placenta, Retained; Lymphoma, AIDS-Related; Tooth Attrition; Hypothalamic Obesity; Schistosomiasis haematobia; Neuralgia; Amyloidosis; Carcinoid Tumor; Dacryocystitis; Familial Hemiplegic Migraine Type 1; Pachydermoperiostosis; Labyrinthitis; Splenomegaly; Hepatolenticular Degeneration; Puberty, Delayed; Moyamoya Disease; Ventricular Outflow Obstruction; Autosomal Recessive Juvenile Parkinson Disease; Chronic Periodontitis; Ectodermal Dysplasia; Glanzmann Thrombasthenia; Placental Insufficiency; Lymphoma, B-Cell; Tooth Avulsion; Hypothermia; Schistosomiasis japonica; Neuralgia, Postherpetic; Amyloidosis AA; Carcinoma; Dandruff; Familial Hypertrophic Cardiomyopathy; Pachyonychia Congenita; Lacerations; Split Spinal Cord Malformation; Hepatomegaly; Puberty, Precocious; Mucinoses; Ventricular Premature Complexes; Autosomal Recessive Polycystic Kidney Disease; Chronic Progressive External Ophthalmoplegia; Ectodermal Dysplasia 1, Anhidrotic; Glaucoma; Plagiocephaly; Lymphoma, B-Cell, Marginal Zone; Tooth Demineralization; Hypothyroidism; Schistosomiasis mansoni; Neurasthenia; Amyloidosis Beta2M; Carcinoma in Situ; Danon Disease; Familial Hypobetalipoproteinemia; Paget Disease of the Breast; Lacrimal Apparatus Diseases; Spondylarthrites; Hepatopulmonary Syndrome; Pubic Symphysis Diastasis; Muckle-Wells Syndrome; Ventricular Remodeling; Autosomal Recessive Spastic Ataxia 4; Chronic Recurrent Multifocal Osteomyelitis; Ectopic Pregnancy; Glaucoma 3 Primary Infantile B; Plagiocephaly, Nonsynostotic; Lymphoma, Extranodal NK-T-Cell; Tooth Discoloration; Hypotrichosis; Schizophrenia; Neurilemmoma; Amyloidosis, Familial; Carcinoma, Acinar Cell; Darier Disease; Familial Hypophosphatemic Rickets; Paget Disease, Extramammary; Lacrimal Duct Obstruction; Spondylarthropathies; Hepatorenal Syndrome; Pudendal Neuralgia; Mucocele; Ventricular Septal Defects; Autosomal Recessive Spinocerebellar Ataxia 9; Chronic Thromboembolic Pulmonary Hypertension; Ectropion; Glaucoma, Angle-Closure; Plague; Lymphoma, Follicular; Tooth Diseases; Hypotrichosis Simplex; Schizophrenia Spectrum and Other Psychotic Disorders; Neuritis; Amyopathic Dermatomyositis; Carcinoma, Adenoid Cystic; DCMA Syndrome; Familial Isolated Hyperparathyroidism; Paget's Disease, Mammary; Lactate Dehydrogenase Deficiency; Spondylitis; Hereditary Amyloidosis; Puerperal Disorders; Mucocutaneous Lymph Node Syndrome; Vernal Keratoconjunctivitis; Avitaminosis; Chronic Traumatic Encephalopathy; Eczema; Glaucoma, Congenital; Plaque, Amyloid; Lymphoma, Large B-Cell, Diffuse; Tooth Erosion; Hypoventilation; Schizophrenia, Catatonic; Neuroacanthocytosis; Amyotrophic Lateral Sclerosis; Carcinoma, Adenosquamous; De Lange Syndrome; Familial Isolated Pituitary Adenoma; Pain; Lactation Disorders; Spondylitis, Ankylosing; Hereditary Angioedema; Puerperal Infection; Mucoepidermoid Carcinoma; Vertebral Artery Dissection; Axenfeld-Rieger Syndrome; Chronobiology Disorders; Eczema, Dyshidrotic; Glaucoma, Neovascular; Plaque, Atherosclerotic; Lymphoma, Large-Cell, Anaplastic; Tooth Eruption, Ectopic; Hypovolemia; Schizophrenia, Childhood; Neuroaspergillosis; etc.

The term “treating” or “treatment” broadly includes any kind of treatment activity, including the diagnosis, cure, mitigation, or prevention of disease in man or other animals, or any activity that otherwise affects the structure or any function of the body of man or other animals.

### Example 1

The effect of varying amounts of potassium carbonate (K₂CO₃) and sodium bicarbonate (NaHCO₃) on the pH of acidic media was tested. The acidic media was chosen to simulate gastric conditions. K₂CO₃ or NaHCO₃ was added to 50 ml of a 0.01 N HCl solution (pH 2). The pH of the solution was measured after addition of the K₂CO₃ or NaHCO₃. Deionized water (240 mL) was then added to the mixture and pH was measured again. The results are shown in Tables 3-6.

TABLE 3
Results with K₂CO₃ (0.01N HCl)

| K₂CO₃ (mg) | pH |
|---|---|
| 25 | 2.84 |
| 35 | 6.29 |
| 45 | 8.05 |
| 50 | 8.29 |
| 100 | 9.43 |
| 200 | 10.14 |
| 300 | 10.39 |
| 400 | 10.55 |
| 450 | 10.58 |

TABLE 4
Results with K₂CO₃ (0.01N HCl + Water)

| K₂CO₃ (mg) | pH |
|---|---|
| 200 | 10.27 |
| 300 | 10.46 |
| 400 | 10.57 |
| 450 | 10.63 |

TABLE 5
Results with NaHCO₃ (0.01N HCl)

| NaHCO₃ (mg) | pH |
|---|---|
| 200 | 5.28 |
| 300 | 5.90 |
| 400 | 6.44 |
| 450 | 6.86 |
| 500 | 8.23 |
| 750 | 8.30 |
| 1000 | 8.36 |

TABLE 6
Results with NaHCO₃ (0.01N HCl + Water)

| NaHCO₃ (mg) | pH |
|---|---|
| 200 | 5.41 |
| 300 | 5.89 |
| 400 | 6.11 |
| 450 | 6.46 |
| 500 | 8.33 |
| 750 | 8.54 |
| 1000 | 8.60 |

### Example 2

Tablets containing meloxicam and combinations of cyclodextrin, K₂CO₃, or NaHCO₃ were manufactured and tested for dissolution. Tablets containing meloxicam alone (MOBIC®) were purchased and also tested for dissolution. The tested tablets are listed in Table 7. Meloxicam in the form of meloxicam/cyclodextrin inclusion complexes was used in the tablets containing meloxicam and cyclodextrin. The inclusion complexes were formed by mixing meloxicam and cyclodextrin in an aqueous pH-adjusted solution. The pH of the solution was adjusted using buffering agents. The resulting soluble meloxicam/cyclodextrin inclusion complexes were then spray dried. This spray-dried dispersion was used in the manufacture of the tablets containing cyclodextrin.

TABLE 7
Tablets

| | |
|---|---|
| Tablet A | 15 mg meloxicam + 25 mg K₂CO3 |
| Tablet B | 15 mg meloxicam + 50 mg K₂CO3 |
| Tablet C | 15 mg meloxicam + 100 mg K₂CO3 |
| Tablet D | 15 mg meloxicam + 150 mg K₂CO3 |
| Tablet E | 15 mg meloxicam + 500 mg NaHCO3 |
| Tablet F | 15 mg meloxicam + 100 mg SBEβCD |
| Tablet G | 15 mg meloxicam + 100 mg SBEβCD + 25 mg K₂CO3 |
| Tablet H | 15 mg meloxicam + 100 mg SBEβCD + 50 mg K₂CO3 |
| Tablet I | 15 mg meloxicam + 100 mg SBEβCD + 100 mg K₂CO3 |
| Tablet J | 15 mg meloxicam + 100 mg SBEβCD + 150 mg K₂CO3 |
| Tablet K | 15 mg meloxicam + 100 mg SBEβCD + 500 mg NaHCO3 |
| Tablet L | 15 mg meloxicam (MOBIC ®) |

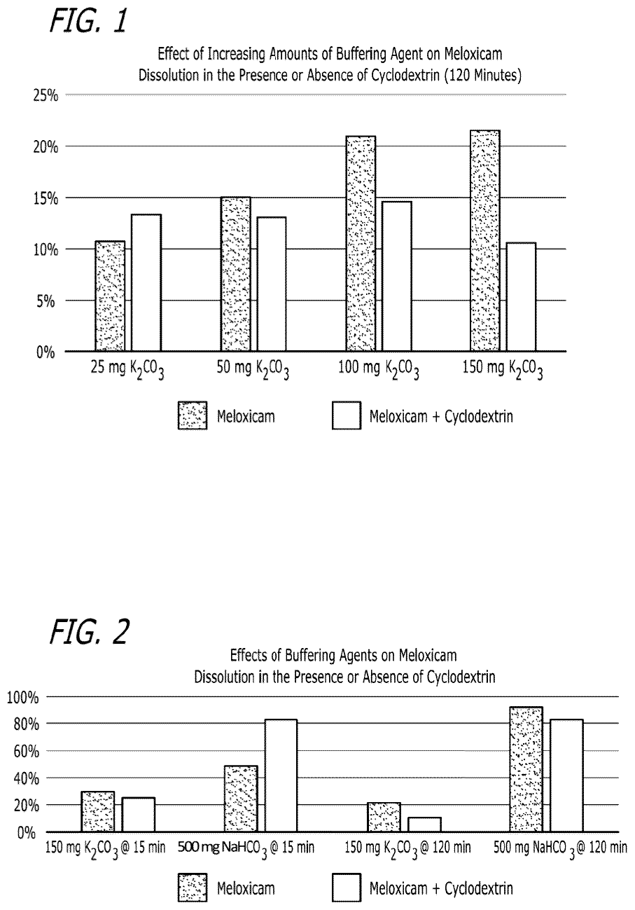

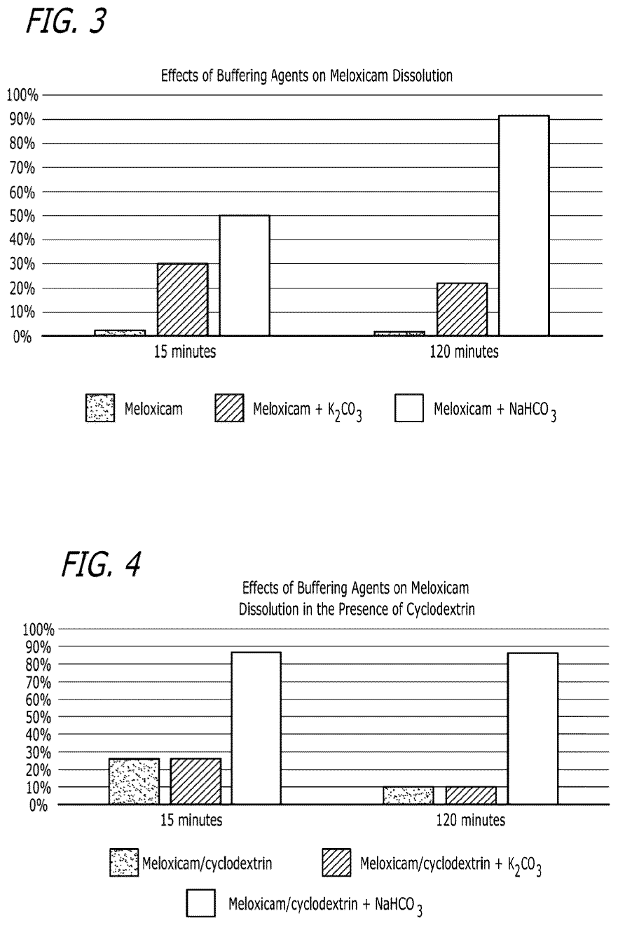

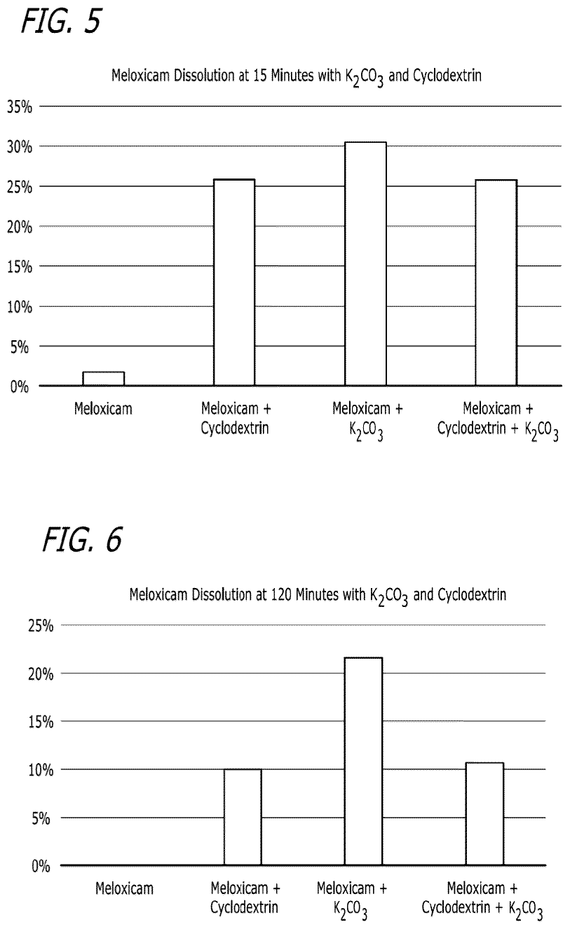

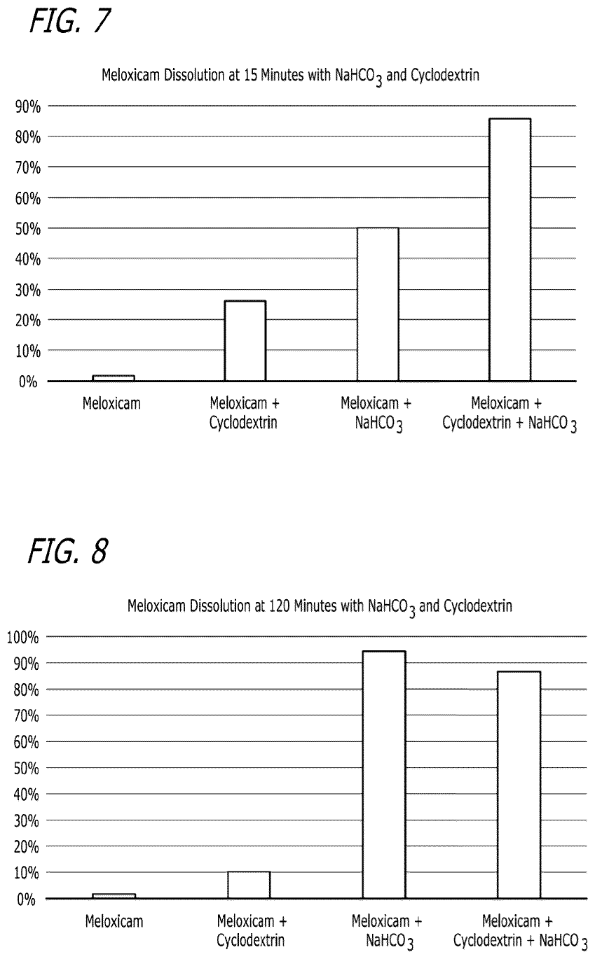

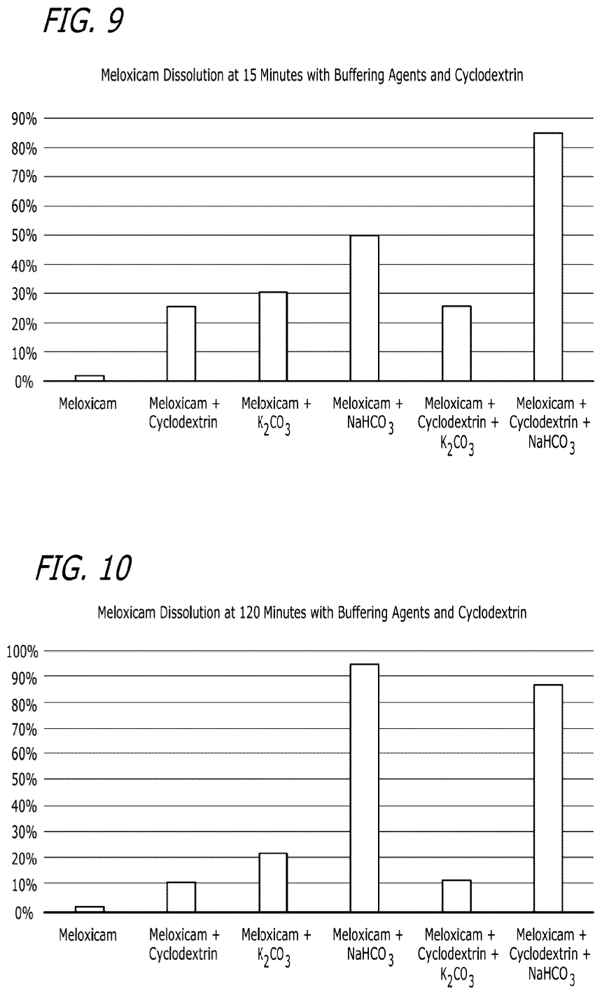

Dissolution testing in acidic medium (chosen to simulate gastric conditions) was performed by placing the tablets in a 0.01 N HCl solution, at an agitation rate of 75 RPM, and vessel temperature of approximately 37° C. The results are presented in Table 8 and in FIGS. 1-10. Results at various time points (0, 15, 30, 45, 60, 90, and 120 minutes) are presented as percent (%) of meloxicam dissolved.

TABLE 8
Dissolution Results

| | 0 | 15 | 30 | 45 | 60 | 90 | 120 |
|---|---|---|---|---|---|---|---|
| | mins | mins | mins | mins | mins | mins | mins |
| Tablet A | 0% | 23% | 17% | 15% | 13% | 12% | 11% |
| Tablet B | 0% | 27% | 20% | 17% | 16% | 17% | 15% |
| Tablet C | 0% | 31% | 26% | 25% | 24% | 23% | 21% |
| Tablet D | 0% | 30% | 26% | 25% | 24% | 23% | 22% |
| Tablet E | 0% | 50% | 66% | 77% | 84% | 92% | 95% |
| Tablet F | 0% | 26% | 17% | 14% | 12% | 11% | 10% |
| Tablet G | 0% | 48% | 39% | 26% | 20% | 16% | 14% |
| Tablet H | 0% | 44% | 30% | 22% | 17% | 16% | 13% |
| Tablet I | 0% | 32% | 33% | 27% | 21% | 16% | 15% |
| Tablet J | 0% | 26% | 27% | 19% | 15% | 12% | 11% |
| Tablet K | 0% | 85% | 86% | 86% | 86% | 86% | 86% |
| Tablet L | 0% | 2% | 2% | 2% | 2% | 2% | 2% |

Dissolution of meloxicam was greater with the tablets containing various combinations of meloxicam and cyclodextrin, K₂CO₃, or NaHCO₃, as compared to tablets containing meloxicam alone. For example, after 120 minutes, dissolution of meloxicam tablets containing NaHCO₃ was 95% as compared to 2% for tablets containing meloxicam alone.

Dissolution of meloxicam increases with increasing amounts of K₂CO₃ in the absence of cyclodextrin. However, in the presence of cyclodextrin, increasing amounts of K₂CO₃ did not appear to increase meloxicam dissolution. At the highest dose of potassium carbonate tested, meloxicam dissolution in the presence of cyclodextrin was reduced by approximately 50% as compared to meloxicam dissolution in the absence of cyclodextrin at 120 minutes.

Dissolution of meloxicam with NaHCO₃ was significantly greater than that observed with the highest dose of K₂CO₃ at 15 minutes (50% versus 30%), and at 120 minutes (92% versus 23%). Meloxicam dissolution in the presence of cyclodextrin was also significantly greater with NaHCO₃ as compared to the highest dose of K₂CO₃ at 15 minutes (85% versus 26%), and at 120 minutes (86% versus 12%). NaHCO₃ in the presence of cyclodextrin increased meloxicam dissolution at 15 minutes as compared to potassium carbonate which resulted in a reduction in dissolution.

### Example 3

A bilayer tablet containing 1) an inclusion complex of SBEβCD with meloxicam, prepared as described below, and 2) sodium bicarbonate that was prepared (SBEβCD-Meloxicam/Bicarbonate). The first layer contained an inclusion complex of 15 mg meloxicam and 100 mg SBEβCD, and 100 mg of sodium bicarbonate. The second layer contained 40 mg of esomeprazole and 400 mg of sodium bicarbonate.

A total of 20 human subjects were randomly assigned in a 1:1 ratio to treatment with the SBEβCD-Meloxicam/Bicarbonate tablets described above or Mobic® tablets (15 mg meloxicam), once daily for 6 days under fasting conditions.

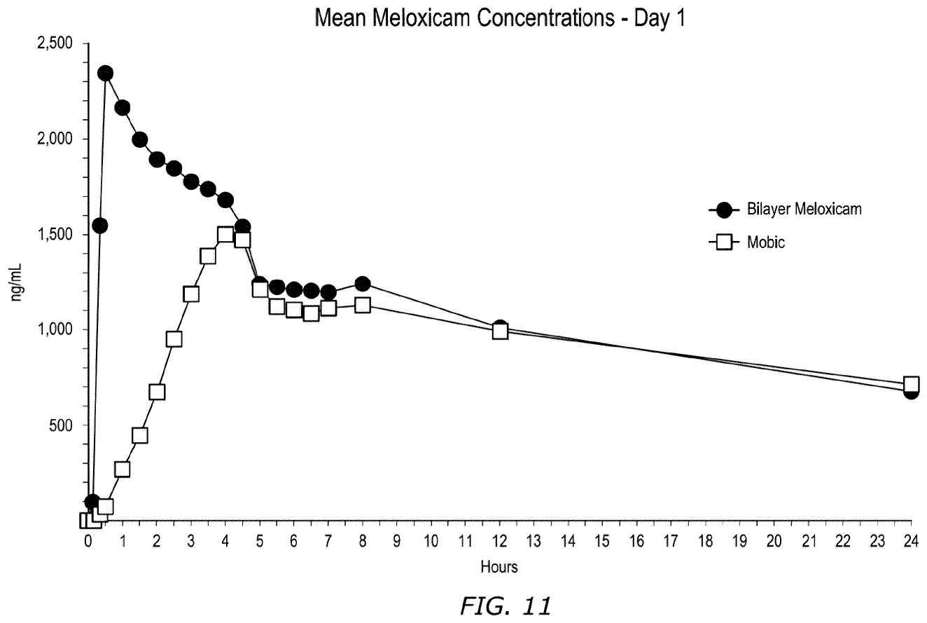

On the first day of dosing, plasma samples were collected for concentration analysis of meloxicam at several time points. Concentrations of meloxicam were determined using LC-MS/MS. Pharmacokinetic parameters were calculated. The results are depicted in FIG. 11.

The median Tₘₐₓ for meloxicam, the trial's primary endpoint, was 9 times faster for the SBEβCD-Meloxicam/Bicarbonate tablets as compared to Mobic® (0.5 hour versus 4.5 hours respectively, p<0.0001).

The SBEβCD-Meloxicam/Bicarbonate tablets also demonstrated higher mean maximum plasma concentration (Cₘₐₓ) (p=0.0018), faster time to therapeutic plasma concentration (p<0.0001), and faster time to half-maximal plasma concentration (p<0.0001) as compared to Mobic®.

Meloxicam in the form of meloxicam/cyclodextrin inclusion complexes was used in the tablets containing meloxicam and cyclodextrin. The inclusion complexes were formed by mixing meloxicam and cyclodextrin in an aqueous pH-adjusted solution. The pH of the solution was adjusted using buffering agents. The resulting soluble meloxicam/cyclodextrin inclusion complexes were then spray dried. This spray-dried dispersion was used in the manufacture of the tablets containing cyclodextrin.

### Example 4

A monolayer tablet containing 1) the inclusion complex of SBEβCD with meloxicam; 2) rizatriptan; and 3) sodium bicarbonate was prepared (SBEβCD-Meloxicam/rizatriptan/Bicarbonate). The monolayer tablet contained 20 mg of meloxicam, 10 mg of rizatriptan, and 500 mg of sodium bicarbonate. The inclusion complex was the same as the inclusion complex of Example 3.

Dissolution testing of the tablets in acidic medium (chosen to simulate gastric conditions) was performed by placing the tablets in a 0.01 N HCl solution, at an agitation rate of 75 RPM, and vessel temperature of approximately 37° C. The results are presented in Table 9. Results at various time points (0, 15, 30, 45, 60, 90, and 120 minutes) are presented as percent (%) of meloxicam, and percent (%) of rizatriptan dissolved.

TABLE 9
Dissolution Results

| Time-point | 0 | 15 | 30 | 45 | 60 | 90 | 120 |
|---|---|---|---|---|---|---|---|
| (minutes) | min | min | min | min | min | min | min |
| Rizatriptan | 0% | 89% | 102% | 103% | 103% | 103% | 103% |
| Meloxicam | 0% | 79% | 92% | 93% | 93% | 93% | 94% |

As shown in Table 9, the dissolution results of the tablets in Example 4 are very similar to the dissolution result of Example 3. Therefore, we expect the pharmacokinetic properties, including bioavailability, Tₘₐₓ of meloxicam, etc., of the tablets in Example 3 to be similar to those described in Example 3 and FIG. 11.

### Example 5

The monolayer tablet of Example 4 was administered to six human subjects. On the first day of dosing, plasma samples were collected for concentration analysis of rizatriptan at several time points. Concentrations of rizatriptan and meloxicam were determined using LC-MS/MS. Pharmacokinetic parameters were calculated. The results for meloxicam were comparable to those reported for the bilayer dosage form of Example 3. The median Tₘₐₓ of rizatriptan was 0.75 hours and the mean Cₘₐₓ of rizatriptan was 20.710 ng/ml. By comparison, the reported Tₘₐₓ of the commercial rizatriptan dosage form, Maxalt®, is 1.0-1.5 hours.

Since both meloxicam and rizatriptan had improved Tₘₐₓ, many other active pharmaceutical ingredients (API) in a dosage form containing a cyclodextrin and/or a bicarbonate may also have improved Tₘₐₓ of the API when orally administering the dosage form described herein to a mammal or a human being in need of treatment with the drug or API. The solubility and bioavailability improvement observed for meloxicam may also occur for the other API in the dosage form that contains a cyclodextrin and/or a bicarbonate. In some embodiments, the cyclodextrin is a sulfobutylether β-cyclodextrin (SBEβCD). In some embodiments, the cyclodextrin is a sulfobutylether β-cyclodextrin (SBEβCD) having about 6 to about 7 sulfobutyl ether groups for each molecule of β-cyclodextrin. In some embodiments, the bicarbonate is sodium bicarbonate. In some embodiments, the bicarbonate is potassium carbonate. In some embodiments, the bicarbonate may improve the dissolution of the API significantly more than the cyclodextrin could when orally administering the dosage form described herein to a mammal or a human being in need of treatment with the API. In some embodiments, the bicarbonate may improve the oral bioavailability of the API significantly more than the cyclodextrin could when orally administering the dosage form described herein to a mammal or a human being in need of treatment with the drug or API.

Unless otherwise indicated, all numbers expressing quantities of ingredients, properties such as molecular weight, reaction conditions, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A solid dosage form comprising a combination of: 1) naproxen or a pharmaceutically acceptable salt thereof, 2) sumatriptan or a pharmaceutically acceptable salt thereof, 3) a cyclodextrin, and 4) a bicarbonate.

2. The solid dosage form of claim 1, comprising about 0.001 mg to about 1000 mg of the naproxen or the pharmaceutically acceptable salt thereof.

3. The solid dosage form of claim 2, comprising about 20 mg to about 600 mg of the naproxen or the pharmaceutically acceptable salt thereof.

4. The solid dosage form of claim 3, comprising about 500 mg of the naproxen or the pharmaceutically acceptable salt thereof.

5. The solid dosage form of claim 3, comprising about 60 mg of the naproxen or the pharmaceutically acceptable salt thereof.

6. The solid dosage form of claim 1, comprising about 0.001 mg to about 1000 mg of the sumatriptan or the pharmaceutically acceptable salt thereof.

7. The solid dosage form of claim 6, comprising about 1 mg to about 200 mg of the sumatriptan or the pharmaceutically acceptable salt thereof.

8. The solid dosage form of claim 7, comprising about 85 mg of the sumatriptan or the pharmaceutically acceptable salt thereof.

9. The solid dosage form of claim 7, comprising about 10 mg of the sumatriptan or the pharmaceutically acceptable salt thereof.

10. The solid dosage form of claim 1, comprising naproxen sodium.

11. The solid dosage form of claim 1, comprising sumatriptan succinate.

12. The solid dosage form of claim 1, comprising 1) about 500 mg of naproxen sodium and 2) about 85 mg of sumatriptan succinate.

13. The solid dosage form of claim 1, comprising 1) about 60 mg of naproxen sodium and 2) about 10 mg of sumatriptan succinate.

14. The solid dosage form of claim 1, wherein the cyclodextrin is a sulfobutylether β-cyclodextrin (SBEβCD).

15. The solid dosage form of claim 14, wherein the SBEβCD has about 6 to about 7 sulfobutyl ether groups for each molecule of β-cyclodextrin.

16. The solid dosage form of claim 15, comprising about 100 mg to about 500 mg of the SBEβCD.

17. The solid dosage form of claim 16, comprising about 100 mg to about 175 mg of the SBEβCD.

18. The solid dosage form of claim 16, comprising about 100 mg to about 140 mg of the SBEβCD.

19. The solid dosage form of claim 1, wherein the bicarbonate comprises sodium bicarbonate.

20. The solid dosage form of claim 1, comprising about 400 mg to about 1000 mg of the bicarbonate.

21. The solid dosage form of claim 19, comprising about 500 mg of sodium bicarbonate.

22. The solid dosage form of claim 1, which is used for the treatment of pain, inflammation, or a related condition, or a combination thereof.
